(19) United States
(12) Patent Application Publication
(10) Pub. No.: US 2014/0080727 A1
(43) Pub. Date: Mar. 20, 2014

(54) VARIANTS PREDICTIVE OF RISK OF GOUT

(71) Applicant: Patrick Sulem, Reykjavik (IS)

(72) Inventor: Patrick Sulem, Reykjavik (IS)

(73) Assignees: Illumina Inc., San Diego, CA (US); deCODE Genetics ehf., Reykjavik (IS)

(21) Appl. No.: 13/984,689

(22) PCT Filed: Feb. 9, 2012
(86) PCT No.: PCT/IS12/50003

§ 371 Date: Nov. 20, 2013

(30) Foreign Application Priority Data
Feb. 9, 2011 (IS) 050007

(51) Int. Cl. C12Q 1/68

(52) U.S. Cl. CPC C12Q 1/6883
USPC 506/9; 435/7.92; 436/501; 506/16; 506/39

(57) ABSTRACT

Markers on chromosome 19q13, in particular, markers in the ALDH16A1 gene, are associated with risk of gout in humans. Diagnostic applications using the markers, such as determining the susceptibility to Gout, are described.

### INTRODUCTION

Gout is a medical condition characterized by recurrent attacks of acute inflammatory arthritis. It is the most common cause of inflammatory arthritis among men and postmenopausal women (Weaver, A. L., CCJM 2008; 75(S5):S9-S12). The pain experienced during an acute attack of gout results from the inflammation that occurs when masses of monosodium urate crystals are deposited into the synovial tissues of a peripheral joint, most often the big toe. Elevated levels of uric acid in the blood are a major risk factor of gout.

Diagnosis is confirmed clinically by the visualization of the characteristic crystals in joint fluid. Treatment of acute attacks includes rest, application of ice to the affected joint, and prescription of colchicine, non-steroidal anti-inflammatory drugs, or both. Once an attack has subsided, levels of uric acid are reduced by lifestyle changes, and for those individuals who experience recurrent attacks, allopurinol or probenicid provide long-term prevention. The aim of urate-lowering therapy is to maintain urate concentration below the saturation point for monosodium urate. Current guidelines recommend that plasma urate should be maintained at a concentration less than 360 μmol/l (EULAR) or 300 μmol/L (British guidelines) (Richette, P. & Bardin, T., Lancet 2010; 375:318-328). When left untreated, acute attacks of gout can lead to chronic gout, characterized by chronic destructive polyarticular involvement with low-grade joint inflammation, joint deformity and tophi, which are monosodium urate crystals surrounded by chronic mononuclear and giant-cell reactions.

The prevalence of gout has increased significantly in recent decades and continues to increase rapidly, owing to the world's enlarging population of elderly individuals, and the proliferation of lifestyles that promote high uric acid levels, including excessive fructose and alcohol intake, physical underactivity and abdominal fat accumation (a hallmark of metabolic syndrome) (VanItallie, T. B., Metabolism Clin Exp 2010; 59(S1):S32-S36). In the USA, the prevalence of men older than 75 years rose from 2.1% in 1990 to 4.1% in 1999 (Wallace, K., et al. J Rheumatol 2004; 31:1582-87).

Occurrence of gout is in part determined by genetics, which are believed to contribute to about 60% of the variability in uric acid levels (Yang, Q., et al., Metabolism 2005; 54:1435-41). A total of eight loci have been reported to be associated with uric acid levels, and variants in the human SLC2A9 and ABCG2 have been reported to be associated with risk of gout (Yang, Q, et al., Circ Cardiovasc Genet. 2010; 3:523-30).

### SUMMARY OF THE INVENTION

The present inventors have discovered that variants on chromosome 19q13 are associated with uric acid levels and risk of gout. In particular, variants, in and near the human ALDH16A1 gene on chromosome 19q13 are associated with risk of gout and uric acid levels. The present invention relates to the utilization of such variants in the risk management of gout.

In a first aspect, the invention provides a method of determining a susceptibility to Gout, the method comprising analyzing sequence data from a human individual for at least one polymorphic marker in the human ALDH16A1 gene, or an encoded ALDH16A1 protein, wherein different alleles of the at least one polymorphic marker are associated with different susceptibilities to Gout in humans, and determining a susceptibility to Gout from the sequence data. In certain embodiments, the sequence data is nucleic acid sequence data.

The invention further provides a method of identification of a marker for use in assessing susceptibility to Gout in human individuals, the method comprising (a) identifying at least one polymorphic marker in the human ALDH16A1 gene; (b) obtaining sequence information about the at least one polymorphic marker in a group of individuals diagnosed with Gout; and (c) obtaining sequence information about the at least one polymorphic marker in a group of control individuals; wherein determination of a significant difference in frequency of at least one allele in the at least one polymorphism in individuals diagnosed with Gout as compared with the frequency of the at least one allele in the control group is indicative of the at least one polymorphism being useful for assessing susceptibility to Gout.

Further provided are prognostic methods and methods of assessing probability to treatment. Thus, a further aspect of the invention relates to a method of predicting prognosis of an individual diagnosed with Gout, the method comprising obtaining sequence data about a human individual about at least one polymorphic marker in the human ALDH16A1 gene, wherein different alleles of the at least one polymorphic marker are associated with different susceptibilities to Gout in humans, and predicting prognosis of Gout from the sequence data. Also provided is a method of assessing probability of response of a human individual to a therapeutic agent for preventing, treating and/or ameliorating symptoms associated with Gout, comprising obtaining sequence data about a human individual identifying at least one allele of at least one polymorphic marker in the human ALDH16A1 gene, wherein different alleles of the at least one polymorphic marker are associated with different probabilities of response to the therapeutic agent in humans, and determining the probability of a positive response to the therapeutic agent from the sequence data.

The invention also provides kits. In one such aspect, the invention relates to a kit for assessing susceptibility to Gout in human individuals, the kit comprising reagents for selectively detecting at least one at-risk variant for Gout in the individual, wherein the at least one at-risk variant is a marker in the human ALDH16A1 gene or an amino acid substitution in an encoded ALDH16A1 protein, and a collection of data comprising correlation data between the at least one at-risk variant and susceptibility to Gout.

Further provided is the use of an oligonucleotide probe in the manufacture of a diagnostic reagent for diagnosing and/or assessing a susceptibility to Gout, wherein the probe is capable of hybridizing to a segment of the human ALDH16A1 gene with sequence as given by SEQ ID NO:4, and wherein the segment is 15-400 nucleotides in length.

The invention also provides computer-implemented applications. In one such application, the invention relates to an apparatus for determining a susceptibility to Gout in a human individual, comprising a processor and a computer readable memory having computer executable instructions adapted to be executed on the processor to analyze information for at least one human individual with respect to at least one marker in the human ALDH16A1 gene, or in an encoded ALDH16A1 protein, that is predictive of susceptibility to Gout in humans, or at least one amino acid substitution in an encoded ALDH16A1 protein, and generate an output based on the marker or amino acid information, wherein the output comprises at least one measure of susceptibility to Gout for the human individual.

It should be understood that all combinations of features described herein are contemplated, even if the combination of feature is not specifically found in the same sentence or paragraph herein. This includes in particular the use of all markers disclosed herein, alone or in combination, for analysis individually or in haplotypes, in all aspects of the invention as described herein.

### BRIEF DESCRIPTION OF THE DRAWINGS

The foregoing and other objects, features and advantages of the invention will be apparent from the following more particular description of preferred embodiments of the invention.

FIG. 1 provides a diagram illustrating a computer-implemented system utilizing risk variants as described herein.

FIG. 2 provides a diagram illustrating a system comprising computer implemented methods utilizing risk variants as described herein.

FIG. 3 shows an exemplary system for determining risk of Gout as described further herein.

FIG. 4 shows a system for selecting a treatment protocol for a subject diagnosed with gout.

### DETAILED DESCRIPTION

### Definitions

Unless otherwise indicated, nucleic acid sequences are written left to right in a 5′ to 3′ orientation. Numeric ranges recited within the specification are inclusive of the numbers defining the range and include each integer or any non-integer fraction within the defined range. Unless defined otherwise, all technical and scientific terms used herein have the same meaning as commonly understood by the ordinary person skilled in the art to which the invention pertains.

The following terms shall, in the present context, have the meaning as indicated:

A “polymorphic marker”, sometime referred to as a “marker”, as described herein, refers to a genomic polymorphic site. Each polymorphic marker has at least two sequence variations characteristic of particular alleles at the polymorphic site. Thus, genetic association to a polymorphic marker implies that there is association to at least one specific allele of that particular polymorphic marker. The marker can comprise any allele of any variant type found in the genome, including SNPs, mini- or microsatellites, translocations and copy number variations (insertions, deletions, duplications). Polymorphic markers can be of any measurable frequency in the population. For mapping of disease genes, polymorphic markers with population frequency higher than 5-10% are in general most useful. However, polymorphic markers may also have lower population frequencies, such as 1-5% frequency, or even lower frequency. The term shall, in the present context, be taken to include polymorphic markers with any population frequency.

An “allele” refers to the nucleotide sequence of a given locus (position) on a chromosome. A polymorphic marker allele thus refers to the composition (i.e., sequence) of the marker on a chromosome. Genomic DNA from an individual contains two alleles (e.g., allele-specific sequences) for any given polymorphic marker, representative of each copy of the marker on each chromosome. Sequence codes for nucleotides used herein are: A=1, C=2, G=3, T=4. For microsatellite alleles, the CEPH sample (Centre d'Etudes du Polymorphisme Humain, genomics repository, CEPH sample 1347-02) is used as a reference, the shorter allele of each microsatellite in this sample is set as 0 and all other alleles in other samples are numbered in relation to this reference. Thus, e.g., allele 1 is 1 bp longer than the shorter allele in the CEPH sample, allele 2 is 2 bp longer than the shorter allele in the CEPH sample, allele 3 is 3 bp longer than the lower allele in the CEPH sample, etc., and allele-1 is 1 bp shorter than the shorter allele in the CEPH sample, allele-2 is 2 bp shorter than the shorter allele in the CEPH sample, etc.

Sequence conucleotide ambiguity as described herein is according to WIPO ST.25:

| IUB code | Meaning |
|---|---|
| A | Adenosine |
| C | Cytidine |
| G | Guanine |
| T | Thymidine |
| R | G or A |
| Y | T or C |
| K | G or T |
| M | A or C |
| S | G or C |
| W | A or T |
| B | C, G or T |
| D | A, G or T |
| H | A, C or T |
| V | A, C or G |
| N | A or G or C or T, unknown or other |

A nucleotide position at which more than one sequence is possible in a population (either a natural population or a synthetic population, e.g., a library of synthetic molecules) is referred to herein as a “polymorphic site”.

A “Single Nucleotide Polymorphism” or “SNP” is a DNA sequence variation occurring when a single nucleotide at a specific location in the genome differs between members of a species or between paired chromosomes in an individual. Most SNP polymorphisms have two alleles. Each individual is in this instance either homozygous for one allele of the polymorphism (i.e. both chromosomal copies of the individual have the same nucleotide at the SNP location), or the individual is heterozygous (i.e. the two sister chromosomes of the individual contain different nucleotides). The SNP nomenclature as reported herein refers to the official Reference SNP (rs) ID identification tag as assigned to each unique SNP by the National Center for Biotechnological Information (NCBI).

A “variant”, as described herein, refers to a segment of DNA that differs from the reference DNA. A “marker” or a “polymorphic marker”, as defined herein, is a variant. Alleles that differ from the reference are referred to as “variant” alleles.

A “microsatellite” is a polymorphic marker that has multiple small repeats of bases that are 2-8 nucleotides in length (such as CA repeats) at a particular site, in which the number of repeat lengths varies in the general population. An “indel” is a common form of polymorphism comprising a small insertion or deletion that is typically only a few nucleotides long.

A “haplotype,” as described herein, refers to a segment of genomic DNA that is characterized by a specific combination of alleles arranged along the segment. For diploid organisms such as humans, a haplotype comprises one member of the pair of alleles for each polymorphic marker or locus along the segment. In a certain embodiment, the haplotype can comprise two or more alleles, three or more alleles, four or more alleles, or five or more alleles.

Allelic identities are described herein in the context of the marker name and the particular allele of the marker, e.g., “3 SG19S921” refers to the 3 allele of marker SG19S921, and is equivalent to “SG19S921 allele 3”. Marker SG19S921 has been assigned the rs number rs150414818 and is therefore equivalent to rs150414818. Thus, “3 rs150414818” refers to the 3 allele of marker rs150414818, and is equivalent to “rs150414818 allele 3”. Furthermore, allelic codes are as for individual markers, i.e. 1=A, 2=C, 3=G and 4=T.

The term “P527R”, in the present context, refers to a proline to arginine substitution at position 527 in the ALDH16A1 protein as set forth in SEQ ID NO:3 herein, which corresponds to the protein with submission ID NP_{—}699160. This amino acid substitution occurs at position 476 in the alternate transcript with submission ID NP_{—}001138868 (SEQ ID NO:2). This substitution is encoded by a C to G substitution in exon 13 of the ALDH16A1 gene (SEQ ID NO:4) (SG19S921; genomic location 54,660,818 bp on chromosome 14 in NCBI Build 36 of the human genome assembly). As described herein, “P527R” refers to the substitution determined by analyzing the protein sequence of the human ALDH16A1 with sequence as set forth in SEQ ID NO:3, or a nucleotide sequence encoding the amino acid at position 527 in the ALDH16A1 protein (i.e., marker SG19S921 (SEQ ID NO:1)).

The term “susceptibility”, as described herein, refers to the proneness of an individual towards the development of a certain state (e.g., a certain trait, phenotype or disease), or towards being less able to resist a particular state than the average individual. The term encompasses both increased susceptibility and decreased susceptibility. Thus, particular alleles at polymorphic markers may be characteristic of increased susceptibility (i.e., increased risk) of Gout, as characterized by a relative risk (RR) or odds ratio (OR) of greater than one for the particular allele. Alternatively, the markers and/or haplotypes of the invention are characteristic of decreased susceptibility (i.e., decreased risk) of Gout, as characterized by a relative risk of less than one.

The term “and/or” shall in the present context be understood to indicate that either or both of the items connected by it are involved. In other words, the term herein shall be taken to mean “one or the other or both”.

The term “look-up table”, as described herein, is a table that correlates one form of data to another form, or one or more forms of data to a predicted outcome to which the data is relevant, such as phenotype or trait. For example, a look-up table can comprise a correlation between allelic data for at least one polymorphic marker and a particular trait or phenotype, such as a particular disease diagnosis, that an individual who comprises the particular allelic data is likely to display, or is more likely to display than individuals who do not comprise the particular allelic data. Look-up tables can be multidimensional, i.e. they can contain information about multiple alleles for single markers simultaneously, or they can contain information about multiple markers, and they may also comprise other factors, such as particulars about diseases diagnoses, racial information, biomarkers, biochemical measurements, therapeutic methods or drugs, etc.

A “computer-readable medium”, is an information storage medium that can be accessed by a computer using a commercially available or custom-made interface. Exemplary computer-readable media include memory (e.g., RAM, ROM, flash memory, etc.), optical storage media (e.g., CD-ROM), magnetic storage media (e.g., computer hard drives, floppy disks, etc.), punch cards, or other commercially available media. Information may be transferred between a system of interest and a medium, between computers, or between computers and the computer-readable medium for storage or access of stored information. Such transmission can be electrical, or by other available methods, such as IR links, wireless connections, etc.

A “nucleic acid sample” as described herein, refers to a sample obtained from an individual that contains nucleic acid (DNA or RNA). In certain embodiments, i.e. the detection of specific polymorphic markers and/or haplotypes, the nucleic acid sample comprises genomic DNA. Such a nucleic acid sample can be obtained from any source that contains genomic DNA, including a blood sample, sample of amniotic fluid, sample of cerebrospinal fluid, or tissue sample from skin, muscle, buccal or conjunctival mucosa, placenta, gastrointestinal tract or other organs.

The term “antisense agent” or “antisense oligonucleotide” refers, as described herein, to molecules, or compositions comprising molecules, which include a sequence of purine an pyrimidine heterocyclic bases, supported by a backbone, which are effective to hydrogen bond to a corresponding contiguous bases in a target nucleic acid sequence. The backbone is composed of subunit backbone moieties supporting the purine and pyrimidine heterocyclic bases at positions which allow such hydrogen bonding. These backbone moieties are cyclic moieties of 5 to 7 atoms in size, linked together by phosphorous-containing linkage units of one to three atoms in length. In certain preferred embodiments, the antisense agent comprises an oligonucleotide molecule.

The term “ALDH16A1”, as described herein, refers to the aldehyde dehydrogenase family 16 member A1 gene on chromosome 19q13. The gene encodes an aldehyde dehydrogenase protein denoted member A1 of family 16.

### Variants on Chromosome 19q13 Associated with Gout

It has been discovered that variants on chromosome 19q13 are predictive of risk of gout in humans. The inventors have identified a novel missense variant in the ALDH16A1 gene that confers significantly increased risk of gout. This missense variant, denoted SG19S921, encodes a Proline to Arginine substitution in human ALDH16A1. This substitution occurs at position 527 in the ALDH16A1 protein as set forth in SEQ ID NO:3 herein, which corresponds to the protein with submission ID NP_{—}699160. In the alternate transcript with submission ID NP_{—}001138868 (SEQ ID NO:2), the substitution occurs at position 476. The substitution is encoded by a C to G substitution in SG19S921 (SEQ ID NO:1). The variant is located in exon 13 of the ALDH16A1 gene, in position 12,534 of the sequence of the ALDH16A1 gene as set forth in SEQ ID NO:4 herein, and has an allelic frequency of 1.9% in the general population and about 5.6% in individuals with gout, which corresponds to an Odds Ratio (OR) of 3.5 and a P-value of 1.50×10⁻¹². The lifetime risk of being diagnosed with gout is estimated to be 3-5% for non-carriers of the P527R variant compared to approximately 30% for carriers of the mutation.

The P527R variant, and other markers in the chromosome 19q13 region have also been found to be significantly associated with uric acid levels in humans. Thus, markers in this region are both predictive of uric acid, which is the main risk factor of Gout, and the disease itself.

The identification of these risk variants, including P527R, thus provides evidence for the role of a rare variant in the development of a common complex disease. This finding has a number of important clinical and diagnostic applications.

### Methods of Determining Susceptibility to Gout

Accordingly, the present invention provides a method of determining a susceptibility to gout, the method comprising analyzing sequence data from a human individual for at least one polymorphic marker in the human ALDH16A1 gene; wherein different alleles of the at least one polymorphic marker are associated with different susceptibilities to gout in humans, and determining a susceptibility to gout from the sequence data.

In certain embodiments, the at least one polymorphic marker encodes a defective ALDH16A1 protein. The defect in ALDH16A1 may for example be a missense substitution, a nonsense substitution or a truncation in ALDH16A1.

In certain embodiments, the data that is obtained is nucleic acid sequence data. In certain embodiments, the nucleic acid sequence data is obtained from a biological sample containing nucleic acid from the human individual. The nucleic acids sequence may suitably be obtained using a method that comprises at least one procedure selected from (i) amplification of nucleic acid from the biological sample; (ii) hybridization assay using a nucleic acid probe and nucleic acid from the biological sample; (iii) hybridization assay using a nucleic acid probe and nucleic acid obtained by amplification of the biological sample, and (iv) sequencing, in particular high-throughput sequencing. The nucleic acid sequence data may also be obtained from a preexisting record. For example, the preexisting record may comprise a genotype dataset for at least one polymorphic marker. In certain embodiments, the determining comprises comparing the sequence data to a database containing correlation data between the at least one polymorphic marker and susceptibility to gout.

In one embodiment, the at least one polymorphic marker is SG19S921 (SEQ ID NO:1). In another embodiment, the at least one polymorphic marker is a marker in linkage disequilibrium with SG19S921. In a preferred embodiment, the at least one polymorphic marker is a marker in linkage disequilibrium with SG19S921 characterized by values of the linkage disequilibrium measure r² of greater than 0.5. In another preferred embodiment, the at least one polymorphic marker is a marker in linkage disequilibrium with SG19S921 characterized by values of the linkage disequilibrium measure r² of greater than 0.8. In another embodiment, the at least one marker encodes an amino acid substitution in an encoded ALDH16A1 protein. In another embodiment, the amino acid substitution is the P527R substitution.

The G allele of SG19S921 is indicative of increased risk of gout. Thus, in certain embodiment, determination of the presence of the G allele of SG19S921 is indicative of increased risk of gout for the individual. Determination of the absence of the G allele of SG19S921 is indicative that the individual does not have the increased risk conferred by the allele.

Alternatively, the allele that is detected can be the allele of the complementary strand of DNA, such that the nucleic acid sequence data identifies at least one allele which is complementary to any of the alleles of the polymorphic markers referenced above. For example, the allele that is detected may be the complementary C allele of the at-risk G allele of SG19S921.

In certain embodiments, the sequence data that is obtained is amino acid sequence data. In certain embodiments, amino acid sequence data comprises data that identifies the amino acid at position 527 in a sequence as set forth in SEQ ID NO:3 herein. In certain embodiments, determination of the presence of a Proline to Arginine substitution at position 527 in a polypeptide with sequence as set forth in SEQ ID NO:3 herein, or in position 476 in a polypeptide with sequence as set forth in SEQ ID NO:2 herein, is indicative of increased susceptibility of gout.

In another aspect, the invention provides a method of determining a susceptibility to Gout, comprising analyzing nucleic acid sequence data for at least one marker selected from the group consisting of

C/G polymorphism at position 54,660,818 in NCBI Build 36 (SEQ ID NO:1);

C/T polymorphism at position 54,676,721 in NCBI Build 36 (SEQ ID NO:5)

A/G polymorphism at position 54,812,310 in NCBI Build 36 (SEQ ID NO:6)

C/G polymorphism at position 54,788,061 in NCBI Build 36 (SEQ ID NO:7)

A/G polymorphism at position 54,818,578 in NCBI Build 36 (SEQ ID NO:8)

C/G polymorphism at position 54,628,061 in NCBI Build 36 (SEQ ID NO:9)

A/C polymorphism at position 54,505,919 in NCBI Build 36 (SEQ ID NO:10)

C/T polymorphism at position 55,483,086 in NCBI Build 36 (SEQ ID NO:11)

C/T polymorphism at position 55,268,031 in NCBI Build 36 (SEQ ID NO:12)

C/T polymorphism at position 55,576,372 in NCBI Build 36 (SEQ ID NO:13)

C/T polymorphism at position 55,456,500 in NCBI Build 36 (SEQ ID NO:14)

A/G polymorphism at position 54,991,872 in NCBI Build 36 (SEQ ID NO:15)

G/T polymorphism at position 55,071,043 in NCBI Build 36 (SEQ ID NO:16)

C/G polymorphism at position 55,071,103 in NCBI Build 36 (SEQ ID NO:17)

C/T polymorphism at position 55,068,782 in NCBI Build 36 (SEQ ID NO:18)

A/G polymorphism at position 55,018,776 in NCBI Build 36 (SEQ ID NO:19)

A/C polymorphism at position 55,471,711 in NCBI Build 36 (SEQ ID NO:20),

or a marker in linkage disequilibrium therewith, wherein different alleles of the at least one polymorphic marker are associated with different susceptibilities to Gout in humans.

In certain embodiments, determination of the presence of a G allele in the C/G polymorphism at position 54,660,818 in NCBI Build 36 (SEQ ID NO:1); a C allele in the C/T polymorphism at position 54,676,721 in NCBI Build 36 (SEQ ID NO:5); a G allele in the A/G polymorphism at position 54,812,310 in NCBI Build 36 (SEQ ID NO:6); a C allele in the C/G polymorphism at position 54,788,061 in NCBI Build 36 (SEQ ID NO:7); an A allele in the A/G polymorphism at position 54,818,578 in NCBI Build 36 (SEQ ID NO:8); a C allele in the C/G polymorphism at position 54,628,061 in NCBI Build 36 (SEQ ID NO:9); a C allele in the A/C polymorphism at position 54,505,919 in NCBI Build 36 (SEQ ID NO:10); a C allele in the C/T polymorphism at position 55,483,086 in NCBI Build 36 (SEQ ID NO:11); a C allele in the C/T polymorphism at position 55,268,031 in NCBI Build 36 (SEQ ID NO:12); a T allele in the C/T polymorphism at position 55,576,372 in NCBI Build 36 (SEQ ID NO:13); a T allele in the C/T polymorphism at position 55,456,500 in NCBI Build 36 (SEQ ID NO:14); a G allele in the A/G polymorphism at position 54,991,872 in NCBI Build 36 (SEQ ID NO:15); a G allele in the G/T polymorphism at position 55,071,043 in NCBI Build 36 (SEQ ID NO:16); a C allele in the C/G polymorphism at position 55,071,103 in NCBI Build 36 (SEQ ID NO:17); a T allele in the C/T polymorphism at position 55,068,782 in NCBI Build 36 (SEQ ID NO:18); an A allele in the A/G polymorphism at position 55,018,776 in NCBI Build 36 (SEQ ID NO:19), and a C allele in the A/C polymorphism at position 55,471,711 in NCBI Build 36 (SEQ ID NO:20) is indicative of increased susceptibility to gout in the human individual. Individuals who are determined to be homozygous for any of these foregoing alleles are at particularly high susceptibility to gout.

It is contemplated that in certain embodiments of the invention, it may be convenient to prepare a report of results of risk assessment. Thus, certain embodiments of the methods of the invention comprise a further step of preparing a report containing results from the determination, wherein said report is written in a computer readable medium, printed on paper, or displayed on a visual display. In certain embodiments, it may be convenient to report results of susceptibility to at least one entity selected from the group consisting of the individual, a guardian of the individual, a genetic service provider, a physician, a medical organization, and a medical insurer.

### Methods of Predicting Levels of Uric Acid

The present inventors have also discovered that markers on chromosome 19q13 are predictive of uric acid levels in humans. Elevated uric acid levels are a major risk factor of gout, and variants that are predictive of elevated uric acid levels are therefore likely to be markers of increased risk of gout. In fact, the markers identified on chromosome 19q13 are indicative of both uric acid levels and gout.

Accordingly, the present invention in a further aspect provides a method of predicting a susceptibility of elevated uric acid levels in humans, the method comprising analyzing sequence data from a human individual about at least one polymorphic marker in the individual, wherein different alleles of the polymorphic marker are indicative of different susceptibilities to elevated uric acid levels, and determining a susceptibility to elevated uric acid levels from the sequence data, wherein the at least one polymorphic marker is selected from the group consisting of

C/G polymorphism at position 54,660,818 in NCBI Build 36 (SEQ ID NO:1);

C/T polymorphism at position 54,676,721 in NCBI Build 36 (SEQ ID NO:5)

A/G polymorphism at position 54,812,310 in NCBI Build 36 (SEQ ID NO:6

C/G polymorphism at position 54,788,061 in NCBI Build 36 (SEQ ID NO:7)

A/G polymorphism at position 54,818,578 in NCBI Build 36 (SEQ ID NO:8)

C/G polymorphism at position 54,628,061 in NCBI Build 36 (SEQ ID NO:9)

A/C polymorphism at position 54,505,919 in NCBI Build 36 (SEQ ID NO:10)

C/T polymorphism at position 55,483,086 in NCBI Build 36 (SEQ ID NO:11)

C/T polymorphism at position 55,268,031 in NCBI Build 36 (SEQ ID NO:12)

C/T polymorphism at position 55,576,372 in NCBI Build 36 (SEQ ID NO:13)

C/T polymorphism at position 55,456,500 in NCBI Build 36 (SEQ ID NO:14)

A/G polymorphism at position 54,991,872 in NCBI Build 36 (SEQ ID NO:15)

G/T polymorphism at position 55,071,043 in NCBI Build 36 (SEQ ID NO:16)

C/G polymorphism at position 55,071,103 in NCBI Build 36 (SEQ ID NO:17)

C/T polymorphism at position 55,068,782 in NCBI Build 36 (SEQ ID NO: 18)

A/G polymorphism at position 55,018,776 in NCBI Build 36 (SEQ ID NO:19)

A/C polymorphism at position 55,471,711 in NCBI Build 36 (SEQ ID NO:20),

C/T polymorphism at position 54,400,812 in NCBI Build 36 (SEQ ID NO:21),

A/G polymorphism at position 53,274,728 in NCBI Build 36 (SEQ ID NO:22), and

A/C polymorphism at position 55,602,702 in NCBI Build 36 (SEQ ID NO:23).

In one embodiment, determination of the presence of a G allele in the C/G polymorphism at position 54,660,818 in NCBI Build 36 (SEQ ID NO:1); a C allele in the C/T polymorphism at position 54,676,721 in NCBI Build 36 (SEQ ID NO:5); a G allele in the A/G polymorphism at position 54,812,310 in NCBI Build 36 (SEQ ID NO:6); a C allele in the C/G polymorphism at position 54,788,061 in NCBI Build 36 (SEQ ID NO:7); an A allele in the A/G polymorphism at position 54,818,578 in NCBI Build 36 (SEQ ID NO:8); a C allele in the C/G polymorphism at position 54,628,061 in NCBI Build 36 (SEQ ID NO:9); a C allele in the A/C polymorphism at position 54,505,919 in NCBI Build 36 (SEQ ID NO:10); a C allele in the C/T polymorphism at position 55,483,086 in NCBI Build 36 (SEQ ID NO:11); a C allele in the C/T polymorphism at position 55,268,031 in NCBI Build 36 (SEQ ID NO:12); a T allele in the C/T polymorphism at position 55,576,372 in NCBI Build 36 (SEQ ID NO:13); a T allele in the C/T polymorphism at position 55,456,500 in NCBI Build 36 (SEQ ID NO:14); a G allele in the A/G polymorphism at position 54,991,872 in NCBI Build 36 (SEQ ID NO:15); a G allele in the G/T polymorphism at position 55,071,043 in NCBI Build 36 (SEQ ID NO:16); a C allele in the C/G polymorphism at position 55,071,103 in NCBI Build 36 (SEQ ID NO:17); a T allele in the C/T polymorphism at position 55,068,782 in NCBI Build 36 (SEQ ID NO:18); an A allele in the A/G polymorphism at position 55,018,776 in NCBI Build 36 (SEQ ID NO:19); a C allele in the A/C polymorphism at position 55,471,711 in NCBI Build 36 (SEQ ID NO:20); a T allele in the C/T polymorphism at position 54,400,812 in NCBI Build 36 (SEQ ID NO:21 a G allele in the A/G polymorphism at position 53,274,728 in NCBI Build 36 (SEQ ID NO:22); and a G allele in the A/C polymorphism at position 55,602,702 in NCBI Build 36 (SEQ ID NO:23) is indicative of susceptibility to elevated uric acid levels in the human individual. Individuals who are determined to be homozygous for any of these foregoing alleles are at particularly high susceptibility to elevated uric acid levels.

In another aspect, the invention provides a method of predicting a susceptibility of elevated uric acid levels in humans, the method comprising analyzing sequence data from a human individual about at least one polymorphic marker in the human ALDH16A1 gene, or an encoded ALDH16A1 protein, in the individual, wherein different alleles of the polymorphic marker are indicative of different susceptibilities to elevated uric acid levels, and determining a susceptibility to elevated uric acid levels from the sequence data. In one embodiment, the at least one polymorphic marker is SG19S921. In another embodiment, the at least one polymorphic marker is a marker in linkage disequilibrium with SG19S921. In another embodiment, the at least one marker is an amino acid substitution in an encoded ALDH16A1 protein. In another embodiment, the at least one marker is a truncation in an encoded ALDH16A1 protein. In another embodiment, the at least one marker is a nonsense substitution in an encoded ALDH16A1 protein. In another embodiment, the amino acid substitution is the P527R substitution.

### Obtaining Nucleic Acid Sequence Data

Sequence data can be nucleic acid sequence data, which may be obtained by means known in the art. Sequence data is suitably obtained from a biological sample of genomic DNA, RNA, or cDNA (a “test sample”) from an individual (“test subject). For example, nucleic acid sequence data may be obtained through direct analysis of the sequence of the polymorphic position (allele) of a polymorphic marker. Suitable methods, some of which are described herein, include, for instance, whole genome sequencing methods, whole genome analysis using SNP chips (e.g., Infinium HD BeadChip), cloning for polymorphisms, non-radioactive PCR-single strand conformation polymorphism analysis, denaturing high pressure liquid chromatography (DHPLC), DNA hybridization, computational analysis, single-stranded conformational polymorphism (SSCP), restriction fragment length polymorphism (RFLP), automated fluorescent sequencing; clamped denaturing gel electrophoresis (CDGE); denaturing gradient gel electrophoresis (DGGE), mobility shift analysis, restriction enzyme analysis; heteroduplex analysis, chemical mismatch cleavage (CMC), RNase protection assays, use of polypeptides that recognize nucleotide mismatches, such as E. coli mutS protein, allele-specific PCR, and direct manual and automated sequencing. These and other methods are described in the art (see, for instance, Li et al., Nucleic Acids Research, 28(2): el (i-v) (2000); Liu et al., Biochem Cell Bio 80:17-22 (2000); and Burczak et al., Polymorphism Detection and Analysis, Eaton Publishing, 2000; Sheffield et al., Proc. Natl. Acad. Sci. USA, 86:232-236 (1989); Orita et al., Proc. Natl. Acad. Sci. USA, 86:2766-2770 (1989); Flavell et al., Cell, 15:25-41 (1978); Geever et al., Proc. Natl. Acad. Sci. USA, 78:5081-5085 (1981); Cotton et al., Proc. Natl. Acad. Sci. USA, 85:4397-4401 (1985); Myers et al., Science 230:1242-1246 (1985); Church and Gilbert, Proc. Natl. Acad. Sci. USA, 81:1991-1995 (1988); Sanger et al., Proc. Natl. Acad. Sci. USA, 74:5463-5467 (1977); and Beavis et al., U.S. Pat. No. 5,288,644).

Recent technological advances have resulted in technologies that allow massive parallel sequencing to be performed in relatively condensed format. These technologies share sequencing-by-synthesis principle for generating sequence information, with different technological solutions implemented for extending, tagging and detecting sequences. Exemplary technologies include 454 pyrosequencing technology (Nyren, P. et al. Anal Biochem 208:171-75 (1993); http://www.454.com), Illumina Solexa sequencing technology (Bentley, D. R. Curr Opin Genet Dev 16:545-52 (2006); http://www.illumina.com), and the SOLID technology developed by Applied Biosystems (ABI) (http://www.appliedbiosystems.com; see also Strausberg, R. L., et al. Drug Disc Today 13:569-77 (2008)). Other sequencing technologies include those developed by Pacific Biosciences (http://www.pacificbiosciences.com), Complete Genomics (http://www.completegenomics.com), Intelligen Bio-Systems (http://www.intelligentbiosystems.com), Genome Corp (http://www.genomecorp.com), ION Torrent Systems (http://www.iontorrent.com) and Helicos Biosciences (http://www.helicosbio.som). It is contemplated that sequence data useful for performing the present invention may be obtained by any such sequencing method, or other sequencing methods that are developed or made available. Thus, any sequence method that provides the allelic identity at particular polymorphic sites (e.g., the absence or presence of particular alleles at particular polymorphic sites) is useful in the methods described and claimed herein.

Alternatively, hybridization methods may be used (see Current Protocols in Molecular Biology, Ausubel et al., eds., John Wiley & Sons, including all supplements). For example, a biological sample of genomic DNA, RNA, or cDNA (a “test sample”) may be obtained from a test subject. The subject can be an adult, child, or fetus. The DNA, RNA, or cDNA sample is then examined. The presence of a specific marker allele can be indicated by sequence-specific hybridization of a nucleic acid probe specific for the particular allele. The presence of more than one specific marker allele or a specific haplotype can be indicated by using several sequence-specific nucleic acid probes, each being specific for a particular allele. A sequence-specific probe can be directed to hybridize to genomic DNA, RNA, or cDNA. A “nucleic acid probe”, as used herein, can be a DNA probe or an RNA probe that hybridizes to a complementary sequence. One of skill in the art would know how to design such a probe so that sequence specific hybridization will occur only if a particular allele is present in a genomic sequence from a test sample.

In certain embodiments, determination of a susceptibility to gout comprises forming a hybridization sample by contacting a test sample, such as a genomic DNA sample, with at least one nucleic acid probe. A non-limiting example of a probe for detecting mRNA or genomic DNA is a labeled nucleic acid probe that is capable of hybridizing to mRNA or genomic DNA sequences described herein. The nucleic acid probe can be, for example, a full-length nucleic acid molecule, or a portion thereof, such as an oligonucleotide of at least 10, 15, 30, 50, 100, 250 or 500 nucleotides in length that is sufficient to specifically hybridize under stringent conditions to appropriate mRNA or genomic DNA. For example, the nucleic acid probe can comprise all or a portion of the nucleotide sequence of the ALDH16A1 gene, or the probe can be the complementary sequence of such a sequence. Hybridization can be performed by methods well known to the person skilled in the art (see, e.g., Current Protocols in Molecular Biology, Ausubel et al., eds., John Wiley & Sons, including all supplements). In one embodiment, hybridization refers to specific hybridization, i.e., hybridization with no mismatches (exact hybridization). In one embodiment, the hybridization conditions for specific hybridization are high stringency.

Specific hybridization, if present, is detected using standard methods. If specific hybridization occurs between the nucleic acid probe and the nucleic acid in the test sample, then the sample contains the allele that is complementary to the nucleotide that is present in the nucleic acid probe.

Additionally, or alternatively, a peptide nucleic acid (PNA) probe can be used in addition to, or instead of, a nucleic acid probe in the hybridization methods described herein. A PNA is a DNA mimic having a peptide-like, inorganic backbone, such as N-(2-aminoethyl)glycine units, with an organic base (A, G, C, T or U) attached to the glycine nitrogen via a methylene carbonyl linker (see, for example, Nielsen et al., Bioconjug. Chem. 5:3-7 (1994)). The PNA probe can be designed to specifically hybridize to a molecule in a sample suspected of containing one or more of the marker alleles shown herein to be associated with risk of gout.

In one embodiment of the invention, a test sample containing genomic DNA obtained from the subject is collected and the polymerase chain reaction (PCR) is used to amplify a fragment comprising one or more polymorphic marker. As described herein, identification of particular marker alleles can be accomplished using a variety of methods. In another embodiment, determination of a susceptibility is accomplished by expression analysis, for example using quantitative PCR (kinetic thermal cycling). This technique can, for example, utilize commercially available technologies, such as TaqMan® (Applied Biosystems, Foster City, Calif.). The technique can for example assess the presence of an alteration in the expression or composition of a polypeptide or splicing variant(s) that is encoded by a nucleic acid associated described herein. Alternatively, this technique may assess expression levels of genes or particular splice variants of genes, that are affected by one or more of the variants described herein. Further, the expression of the variant(s) can be quantified as physically or functionally different.

Allele-specific oligonucleotides can also be used to detect the presence of a particular allele in a nucleic acid. An “allele-specific oligonucleotide” (also referred to herein as an “allele-specific oligonucleotide probe”) is an oligonucleotide of any suitable size, for example an oligonucleotide of approximately 10-50 base pairs or approximately 15-30 base pairs, that specifically hybridizes to a nucleic acid which contains a specific allele at a polymorphic site (e.g., a polymorphic marker). An allele-specific oligonucleotide probe that is specific for one or more particular alleles at polymorphic markers can be prepared using standard methods (see, e.g., Current Protocols in Molecular Biology, supra). PCR can be used to amplify the desired region. Specific hybridization of an allele-specific oligonucleotide probe to DNA from a subject is indicative of the presence of a specific allele at a polymorphic site (see, e.g., Gibbs et al., Nucleic Acids Res. 17:2437-2448 (1989) and WO 93/22456).

With the addition of analogs such as locked nucleic acids (LNAs), the size of primers and probes can be reduced to as few as 8 bases. LNAs are a novel class of bicyclic DNA analogs in which the 2′ and 4′ positions in the furanose ring are joined via an O-methylene (oxy-LNA), S-methylene (thio-LNA), or amino methylene (amino-LNA) moiety. Common to all of these LNA variants is an affinity toward complementary nucleic acids, which is by far the highest reported for a DNA analog. For example, particular all oxy-LNA nonamers have been shown to have melting temperatures (Tm) of 64° C. and 74° C. when in complex with complementary DNA or RNA, respectively, as opposed to 28° C. for both DNA and RNA for the corresponding DNA nonamer. Substantial increases in Tm are also obtained when LNA monomers are used in combination with standard DNA or RNA monomers. For primers and probes, depending on where the LNA monomers are included (e.g., the 3′ end, the 5′ end, or in the middle), the Tm could be increased considerably. It is therefore contemplated that in certain embodiments, LNAs are used to detect particular alleles at polymorphic sites associated with Gout, as described herein.

In certain embodiments, arrays of oligonucleotide probes that are complementary to target nucleic acid sequence segments from a subject can be used to identify polymorphisms in a nucleic acid. For example, an oligonucleotide array can be used. Oligonucleotide arrays typically comprise a plurality of different oligonucleotide probes that are coupled to a surface of a substrate in different known locations. These arrays can generally be produced using mechanical synthesis methods or light directed synthesis methods that incorporate a combination of photolithographic methods and solid phase oligonucleotide synthesis methods, or by other methods known to the person skilled in the art (see, e.g., Bier et al., Adv Biochem Eng Biotechnol 109:433-53 (2008); Hoheisel, Nat Rev Genet. 7:200-10 (2006); Fan et al., Methods Enzymol 410:57-73 (2006); Raqoussis & Elvidge, Expert Rev Mol Diagn 6:145-52 (2006); Mockler et al., Genomics 85:1-15 (2005), and references cited therein, the entire teachings of each of which are incorporated by reference herein). Many additional descriptions of the preparation and use of oligonucleotide arrays for detection of polymorphisms can be found, for example, in U.S. Pat. No. 6,858,394, U.S. Pat. No. 6,429,027, U.S. Pat. No. 5,445,934, U.S. Pat. No. 5,700,637, U.S. Pat. No. 5,744,305, U.S. Pat. No. 5,945,334, U.S. Pat. No. 6,054,270, U.S. Pat. No. 6,300,063, U.S. Pat. No. 6,733,977, U.S. Pat. No. 7,364,858, EP 619 321, and EP 373 203, the entire teachings of which are incorporated by reference herein.

Also, standard techniques for genotyping can be used to detect particular marker alleles, such as fluorescence-based techniques (e.g., Chen et al., Genome Res. 9(5): 492-98 (1999); Kutyavin et al., Nucleic Acid Res. 34:e128 (2006)), utilizing PCR, LCR, Nested PCR and other techniques for nucleic acid amplification. Specific commercial methodologies available for SNP genotyping include, but are not limited to, TaqMan genotyping assays and SNPlex platforms (Applied Biosystems), gel electrophoresis (Applied Biosystems), mass spectrometry (e.g., MassARRAY system from Sequenom), minisequencing methods, real-time PCR, Bio-Plex system (BioRad), CEQ and SNPstream systems (Beckman), array hybridization technology (e.g., Affymetrix GeneChip; Perlegen), BeadArray Technologies (e.g., Illumina GoldenGate and Infinium assays), array tag technology (e.g., Parallele), and endonuclease-based fluorescence hybridization technology (Invader; Third Wave).

Suitable biological sample in the methods described herein can be any sample containing nucleic acid (e.g., genomic DNA) and/or protein from the human individual. For example, the biological sample can be a blood sample, a serum sample, a leukapheresis sample, an amniotic fluid sample, a cerbrospinal fluid sample, a hair sample, a tissue sample from skin, muscle, buccal, or conjuctival mucosa, placenta, gastrointestinal tract, or other organs, a semen sample, a urine sample, a saliva sample, a nail sample, a tooth sample, and the like. Preferably, the sample is a blood sample, a salive sample or a buccal swab.

### Protein Analysis

Missense nucleic acid variations may lead to an altered amino acid sequence, as compared to the non-variant (e.g., wild-type) protein, due to amino acid substitutions, deletions, or insertions, or truncations (due to, e.g., splice variation). In such instances, detection of the amino acid substitution of the variant protein may be useful. This way, nucleic acid sequence data may be obtained through indirect analysis of the nucleic acid sequence of the allele of the polymorphic marker, i.e. by detecting a protein variation. Methods of detecting variant proteins are known in the art. For example, direct amino acid sequencing of the variant protein followed by comparison to a reference amino acid sequence can be used. Alternatively, SDS-PAGE followed by gel staining can be used to detect variant proteins of different molecular weights. Also, Immunoassays, e.g., antibody assays, e.g., immunofluorescent immunoassays, immunoprecipitations, radioimmunoasays, ELISA, and Western blotting, in which an antibody specific for an epitope comprising the variant sequence among the variant protein and non-variant or wild-type protein can be used.

In certain embodiments of the present invention, an amino acid substitution in the human ALDH16A1 protein is detected. In one embodiment, the amino acid substitution is detected in a protein sample from a human individual. In one embodiment, the P527R substitution in human ALDH16A1 is detected in a protein sample. The detection may be suitably performed, for example using any of the methods described in the above, or any other suitable method known to the skilled artisan.

In some cases, a variant protein has altered (e.g., upregulated or downregulated) biological activity, in comparison to the non-variant or wild-type protein. The biological activity can be, for example, a binding activity or enzymatic activity. In this instance, altered biological activity may be used to detect a variation in protein encoded by a nucleic acid sequence variation. Methods of detecting binding activity and enzymatic activity are known in the art and include, for instance, ELISA, competitive binding assays, quantitative binding assays using instruments such as, for example, a Biacore® 3000 instrument, chromatographic assays, e.g., HPLC and TLC.

Alternatively or additionally, a protein variation encoded by a genetic variation could lead to an altered expression level, e.g., an increased expression level of an mRNA or protein, a decreased expression level of an mRNA or protein. In such instances, nucleic acid sequence data about the allele of the polymorphic marker, or protein sequence data about the protein variation, can be obtained through detection of the altered expression level. Methods of detecting expression levels are known in the art. For example, ELISA, radioimmunoassays, immunofluorescence, and Western blotting can be used to compare the expression of protein levels. Alternatively, Northern blotting can be used to compare the levels of mRNA. These processes are described in Sambrook et al., Molecular Cloning: A Laboratory Manual, 3^{rd} ed. Cold Spring Harbor Laboratory Press, Cold Spring Harbor, N.Y. (2001).

Any of these methods may be performed using a nucleic acid (e.g., DNA, mRNA) or protein of a biological sample obtained from the human individual for whom a susceptibility is being determined. The biological sample can be any nucleic acid or protein containing sample obtained from the human individual. For example, the biological sample can be any of the biological samples described herein.

### Number of Polymorphic Markers/Genes Analyzed

With regard to the methods of determining a susceptibility described herein, the methods can comprise obtaining sequence data about any number of polymorphic markers and/or about any number of genes. For example, the method can comprise obtaining sequence data for about at least 1, 2, 3, 4, 5, 6, 7, 8, 9, 10, 15, 20, 25, 30, 40, 50, 100, 500, 1000, 10,000 or more polymorphic markers. In certain embodiments, the sequence data is obtained from a microarray comprising probes for detecting a plurality of markers. The markers can be independent of SG19S921 and/or the markers may be in linkage disequilibrium with SG19S921. The polymorphic markers can be the ones of the group specified herein or they can be different polymorphic markers that are not specified herein. In a specific embodiment, the method comprises obtaining sequence data about at least two polymorphic markers. In certain embodiments, each of the markers may be associated with a different gene. For example, in some instances, if the method comprises obtaining nucleic acid data about a human individual identifying at least one allele of a polymorphic marker, then the method comprises identifying at least one allele of at least one polymorphic marker. Also, for example, the method can comprise obtaining sequence data about a human individual identifying alleles of multiple, independent markers, which are not in linkage disequilibrium.

### Linkage Disequilibrium

Linkage Disequilibrium (LD) refers to a non-random assortment of two genetic elements. For example, if a particular genetic element (e.g., an allele of a polymorphic marker, or a haplotype) occurs in a population at a frequency of 0.50 (50%) and another element occurs at a frequency of 0.50 (50%), then the predicted occurrance of a person's having both elements is 0.25 (25%), assuming a random distribution of the elements. However, if it is discovered that the two elements occur together at a frequency higher than 0.25, then the elements are said to be in linkage disequilibrium, since they tend to be inherited together at a higher rate than what their independent frequencies of occurrence (e.g., allele or haplotype frequencies) would predict. Roughly speaking, LD is generally correlated with the frequency of recombination events between the two elements. Allele or haplotype frequencies can be determined in a population by genotyping individuals in a population and determining the frequency of the occurence of each allele or haplotype in the population. For populations of diploids, e.g., human populations, individuals will typically have two alleles for each genetic element (e.g., a marker, haplotype or gene).

Many different measures have been proposed for assessing the strength of linkage disequilibrium (LD; reviewed in Devlin, B. & Risch, N., Genomics 29:311-22 (1995)). Most capture the strength of association between pairs of biallelic sites. Two important pairwise measures of LD are r² (sometimes denoted Δ²) and |D′| (Lewontin, R., Genetics 49:49-67 (1964); Hill, W. G. & Robertson, A. Theor. Appl. Genet. 22:226-231 (1968)). Both measures range from 0 (no disequilibrium) to 1 (‘complete’ disequilibrium), but their interpretation is slightly different. |D′| is defined in such a way that it is equal to 1 if just two or three of the possible haplotypes are present, and it is <1 if all four possible haplotypes are present. Therefore, a value of |D′| that is <1 indicates that historical recombination may have occurred between two sites (recurrent mutation can also cause |D′| to be <1, but for single nucleotide polymorphisms (SNPs) this is usually regarded as being less likely than recombination). The measure r² represents the statistical correlation between two sites, and takes the value of 1 if only two haplotypes are present. Markers which are correlated by an r² value of 1 are said to be perfectly correlated. In such an instance, the genotype of one marker perfectly predicts the genotype of the other.

The r² measure is arguably the most relevant measure for association mapping, because there is a simple inverse relationship between r² and the sample size required to detect association between susceptibility loci and SNPs. These measures are defined for pairs of sites, but for some applications a determination of how strong LD is across an entire region that contains many polymorphic sites might be desirable (e.g., testing whether the strength of LD differs significantly among loci or across populations, or whether there is more or less LD in a region than predicted under a particular model). Measuring LD across a region is not straightforward, but one approach is to use the measure r, which was developed in population genetics. Roughly speaking, r measures how much recombination would be required under a particular population model to generate the LD that is seen in the data. This type of method can potentially also provide a statistically rigorous approach to the problem of determining whether LD data provide evidence for the presence of recombination hotspots.

A significant r² indicative of markers being in linkage disequilibrium may be at least 0.1, such as at least 0.15, 0.2, 0.25, 0.3, 0.35, 0.4, 0.45, 0.5, 0.55, 0.6, 0.65, 0.7, 0.75, 0.8, 0.85, 0.9, 0.91, 0.92, 0.93, 0.94, 0.95, 0.96, 0.97, 0.98, 0.99 or 1.0. In one specific embodiment of invention, the significant r² value can be at least 0.2. In another specific embodiment of invention, the significant r² value can be at least 0.5. In one specific embodiment of invention, the significant r² value can be at least 0.8. Alternatively, linkage disequilibrium as described herein, refers to linkage disequilibrium characterized by values of r² of at least 0.2, such as 0.3, 0.4, 0.5, 0.6, 0.7, 0.8, 0.85, 0.9, 0.95, 0.96, 0.97, 0.98, 0.99. Thus, linkage disequilibrium represents a correlation between alleles of distinct markers. It is measured by correlation coefficient or |D′| (r² up to 1.0 and |D′| up to 1.0). Linkage disequilibrium can be determined in a single human population, as defined herein, or it can be determined in a collection of samples comprising individuals from more than one human population. In one embodiment of the invention, LD is determined in a sample from one or more of the HapMap populations. These include samples from the Yoruba people of Ibadan, Nigeria (YR1), samples from individuals from the Tokyo area in Japan (JPT), samples from individuals Beijing, China (CHB), and samples from U.S. residents with northern and western European ancestry (CEU), as described (The International HapMap Consortium, Nature 426:789-796 (2003)). In one such embodiment, LD is determined in the Caucasian CEU population of the HapMap samples. In another embodiment, LD is determined in the African YRI population. In yet another embodiment, LD is determined in samples from the Icelandic population.

If all polymorphisms in the genome were independent at the population level (i.e., no LD between polymorphisms), then every single one of them would need to be investigated in association studies, to assess all different polymorphic states. However, due to linkage disequilibrium between polymorphisms, tightly linked polymorphisms are strongly correlated, which reduces the number of polymorphisms that need to be investigated in an association study to observe a significant association. Another consequence of LD is that many polymorphisms may give an association signal due to the fact that these polymorphisms are strongly correlated.

Genomic LD maps have been generated across the genome, and such LD maps have been proposed to serve as framework for mapping disease-genes (Risch, N. & Merkiangas, K, Science 273:1516-1517 (1996); Maniatis, N., et al., Proc Natl Acad Sci USA 99:2228-2233 (2002); Reich, D E et al, Nature 411:199-204 (2001)).

It is now established that many portions of the human genome can be broken into series of discrete haplotype blocks containing a few common haplotypes; for these blocks, linkage disequilibrium data provides little evidence indicating recombination (see, e.g., Wall., J. D. and Pritchard, J. K., Nature Reviews Genetics 4:587-597 (2003); Daly, M. et al., Nature Genet. 29:229-232 (2001); Gabriel, S. B. et al., Science 296:2225-2229 (2002); Patil, N. et al., Science 294:1719-1723 (2001); Dawson, E. et al., Nature 418:544-548 (2002); Phillips, M. S. et al., Nature Genet. 33: 382-387 (2003)).

Haplotype blocks (LD blocks) can be used to map associations between phenotype and haplotype status, using single markers or haplotypes comprising a plurality of markers. The main haplotypes can be identified in each haplotype block, and then a set of “tagging” SNPs or markers (the smallest set of SNPs or markers needed to distinguish among the haplotypes) can then be identified. These tagging SNPs or markers can then be used in assessment of samples from groups of individuals, in order to identify association between phenotype and haplotype. If desired, neighboring haplotype blocks can be assessed concurrently, as there may also exist linkage disequilibrium among the haplotype blocks.

It has thus become apparent that for any given observed association to a polymorphic marker in the genome, it is likely that additional markers in the genome also show association. This is a natural consequence of the uneven distribution of LD across the genome, as observed by the large variation in recombination rates. The markers used to detect association thus in a sense represent “tags” for a genomic region (i.e., a haplotype block or LD block) that is associating with a given disease or trait, and as such are useful for use in the methods and kits of the invention.

By way of example, the marker SG19S921, encoding the P527R substitution in human ALDH16A1 may be detected directly to determine risk of gout. Alternatively, any marker in linkage disequilibrium with SG19S921 may be detected to determine risk.

The present invention relates to the SG19S921 for detecting association to gout. Markers in linkage disequilibrium with SG19S921 may also be useful for detecting the association. Thus, in certain embodiments of the invention, markers that are in LD with SG19S921 may be used as surrogate markers.

Suitable surrogate markers may be selected using public information, such as from the International HapMap Consortium (http://www.hapmap.org) and the International 1000 genomes Consortium (http://www.1000genomes.org). The stronger the linkage disequilibrium to the anchor marker, the better the surrogate, and thus the mores similar the association detected by the surrogate is expected to be to the association detected by the anchor marker. Markers with values of r² equal to 1 are perfect surrogates for the at-risk variants, i.e. genotypes for one marker perfectly predicts genotypes for the other. In other words, the surrogate will, by necessity, give exactly the same association data to any particular disease as the anchor marker. Markers with smaller values of r² than 1 can also be surrogates for the at-risk anchor variant.

The present invention encompasses the assessment of such surrogate markers for the markers as disclosed herein. Such markers are annotated, mapped and listed in public databases, as well known to the skilled person, or can alternatively be readily identified by sequencing the region or a part of the region identified by the markers of the present invention in a group of individuals, and identify polymorphisms in the resulting group of sequences. As a consequence, the person skilled in the art can readily and without undue experimentation identify and select appropriate surrogate markers.

One consequence of LD is that causative variants are not necessarily the variants first used for detecting an association signal. It is contemplated that a variant that is in linkage disequilibrium with SG19S921 (which encodes the P527R substitution) may be the functionally relevant variant. Alternatively, one or more variants in linkage disequilibrium with SG19S921 may also be functionally relevant variants predictive of risk of gout. A number of genes are found in the 19q13 region that includes the SG19S921 variant, in addition to ALDH16A1. These include for example the Solute Carrier Family 6, member 16 gene (SLC6A16), and the Solute Carrier Family 17, member 7 gene (SLC17A7). Members of the solute carrier family of proteins have been implicated in both regulation of uric acid levels and risk of gout. It is therefore contemplated that either the SLC6A16 and/or the SLC17A7 genes also harbor variants that predictive of risk of gout. Such variants are useful in the methods as described herein.

### Association Analysis

For single marker association to a disease, the Fisher exact test can be used to calculate two-sided p-values for each individual allele. Correcting for relatedness among patients can be done by extending a variance adjustment procedure previously described (Risch, N. & Teng, J. Genome Res., 8:1273-1288 (1998)) for sibships so that it can be applied to general familial relationships. The method of genomic controls (Devlin, B. & Roeder, K. Biometrics 55:997 (1999)) can also be used to adjust for the relatedness of the individuals and possible stratification.

For both single-marker and haplotype analyses, relative risk (RR) and the population attributable risk (PAR) can be calculated assuming a multiplicative model (haplotype relative risk model) (Terwilliger, J. D. & Ott, J., Hum. Hered. 42:337-46 (1992) and Falk, C. T. & Rubinstein, P, Ann. Hum. Genet. 51 (Pt 3):227-33 (1987)), i.e., that the risks of the two alleles/haplotypes a person carries multiply. For example, if RR is the risk of A relative to a, then the risk of a person homozygote AA will be RR times that of a heterozygote Aa and RR² times that of a homozygote aa. The multiplicative model has a nice property that simplifies analysis and computations—haplotypes are independent, i.e., in Hardy-Weinberg equilibrium, within the affected population as well as within the control population. As a consequence, haplotype counts of the affecteds and controls each have multinomial distributions, but with different haplotype frequencies under the alternative hypothesis. Specifically, for two haplotypes, hᵢ and hⱼ, risk(hᵢ)/risk(hⱼ)=(fᵢ/pᵢ)/(fⱼ/pⱼ), where f and p denote, respectively, frequencies in the affected population and in the control population. While there is some power loss if the true model is not multiplicative, the loss tends to be mild except for extreme cases. Most importantly, p-values are always valid since they are computed with respect to null hypothesis.

### Risk Assessment and Diagnostics

Within any given population, there is an absolute risk of developing a disease or trait, defined as the chance of a person developing the specific disease or trait over a specified time-period. For example, a woman's lifetime absolute risk of breast cancer is one in nine. That is to say, one woman in every nine will develop breast cancer at some point in their lives. Risk is typically measured by looking at very large numbers of people, rather than at a particular individual. Risk is often presented in terms of Absolute Risk (AR) and Relative Risk (RR). Relative Risk is used to compare risks associating with two variants or the risks of two different groups of people. For example, it can be used to compare a group of people with a certain genotype with another group having a different genotype. For a disease, a relative risk of 2 means that one group has twice the chance of developing a disease as the other group. The risk presented is usually the relative risk for a person, or a specific genotype of a person, compared to the population with matched gender and ethnicity. Risks of two individuals of the same gender and ethnicity could be compared in a simple manner. For example, if, compared to the population, the first individual has relative risk 1.5 and the second has relative risk 0.5, then the risk of the first individual compared to the second individual is 1.5/0.5=3.

### Risk Calculations

The creation of a model to calculate the overall genetic risk involves two steps: i) conversion of odds-ratios for a single genetic variant into relative risk and ii) combination of risk from multiple variants in different genetic loci into a single relative risk value.

### Deriving Risk from Odds-Ratios

Most gene discovery studies for complex diseases that have been published to date in authoritative journals have employed a case-control design because of their retrospective setup. These studies sample and genotype a selected set of cases (people who have the specified disease condition) and control individuals. The interest is in genetic variants (alleles) which frequency in cases and controls differ significantly.

The results are typically reported in odds ratios, that is the ratio between the fraction (probability) with the risk variant (carriers) versus the non-risk variant (non-carriers) in the groups of affected versus the controls, i.e. expressed in terms of probabilities conditional on the affection status:

OR=(Pr(c|A)/Pr(nc|A))/(Pr(c|C)/Pr(nc|C))

Sometimes it is however the absolute risk for the disease that we are interested in, i.e. the fraction of those individuals carrying the risk variant who get the disease or in other words the probability of getting the disease. This number cannot be directly measured in case-control studies, in part, because the ratio of cases versus controls is typically not the same as that in the general population. However, under certain assumption, we can estimate the risk from the odds ratio.

It is well known that under the rare disease assumption, the relative risk of a disease can be approximated by the odds ratio. This assumption may however not hold for many common diseases. Still, it turns out that the risk of one genotype variant relative to another can be estimated from the odds ratio expressed above. The calculation is particularly simple under the assumption of random population controls where the controls are random samples from the same population as the cases, including affected people rather than being strictly unaffected individuals. To increase sample size and power, many of the large genome-wide association and replication studies use controls that were neither age-matched with the cases, nor were they carefully scrutinized to ensure that they did not have the disease at the time of the study. Hence, while not exactly, they often approximate a random sample from the general population. It is noted that this assumption is rarely expected to be satisfied exactly, but the risk estimates are usually robust to moderate deviations from this assumption.

Calculations show that for the dominant and the recessive models, where we have a risk variant carrier, “c”, and a non-carrier, “nc”, the odds ratio of individuals is the same as the risk ratio between these variants:

OR=Pr(A|c)/Pr(A|nc)=r

And likewise for the multiplicative model, where the risk is the product of the risk associated with the two allele copies, the allelic odds ratio equals the risk factor:

OR=Pr(A|aa)/Pr(A|ab)=Pr(A|ab)/Pr(A|bb)=r

Here “a” denotes the risk allele and “b” the non-risk allele. The factor “r” is therefore the relative risk between the allele types.

For many of the studies published in the last few years, reporting common variants associated with complex diseases, the multiplicative model has been found to summarize the effect adequately and most often provide a fit to the data superior to alternative models such as the dominant and recessive models.

### Determining Risk

In the present context, an individual who is at an increased susceptibility (i.e., increased risk) for Gout is an individual who is carrying at least one at-risk variant as described herein. In certain embodiments, the variant is within the human ALDH16A1 gene, or a variant encoded by a variation in the human ALDH16A1 gene. In one embodiment, significance associated with a marker is measured by a relative risk (RR). In another embodiment, significance associated with a marker or haplotye is measured by an odds ratio (OR). In a further embodiment, the significance is measured by a percentage. In one embodiment, a significant increased risk is measured as a risk (relative risk and/or odds ratio) of at least 2.0, including but not limited to: at least 2.5, at least 3.0, at least 3.5, at least 4.0, at least 4.5, and at least 5.0. In a particular embodiment, a risk (relative risk and/or odds ratio) of at least 3.0 is significant. In another particular embodiment, a risk of at least 3.5 is significant.

An at-risk variant as described herein is one where at least one allele of at least one marker or haplotype is more frequently present in an individual at risk for Gout (affected), or diagnosed with Gout, compared to the frequency of Gout in a comparison group (control), such that the presence of the marker is indicative of susceptibility to Gout. The control group may in one embodiment be a population sample, i.e. a random sample from the general population. In another embodiment, the control group is represented by a group of individuals who are disease-free, i.e. individuals who have not been diagnosed with Gout.

The person skilled in the art will appreciate that for markers with two alleles present in the population being studied (such as SNPs), and wherein one allele is found in increased frequency in a group of individuals with a trait or disease in the population, compared with controls, the other allele of the marker will be found in decreased frequency in the group of individuals with the trait or disease, compared with controls. In such a case, one allele of the marker (the one found in increased frequency in individuals with the trait or disease) will be the at-risk allele, while the other allele will be a protective allele.

### Database

Determining susceptibility can alternatively or additionally comprise comparing nucleic acid sequence data and/or protein sequence data (genotype data) to a database containing correlation data between polymorphic markers and susceptibility to Gout. The database can be part of a computer-readable medium described herein.

In a specific aspect of the invention, the database comprises at least one measure of susceptibility to Gout for the polymorphic markers. For example, the database may comprise risk values associated with particular genotypes at such markers. The database may also comprise risk values associated with particular genotype combinations for multiple such markers.

In another specific aspect of the invention, the database comprises a look-up table containing at least one measure of susceptibility to Gout for the polymorphic markers.

### Further Steps

The methods disclosed herein can comprise additional steps which may occur before, after, or simultaneously with one of the aforementioned steps of the method of the invention. In a specific embodiment of the invention, the method of determining a susceptibility to Gout further comprises reporting the susceptibility to at least one entity selected from the group consisting of the individual, a guardian of the individual, a genetic service provider, a physician, a medical organization, and a medical insurer. The reporting may be accomplished by any of several means. For example, the reporting can comprise sending a written report on physical media or electronically or providing an oral report to at least one entity of the group, which written or oral report comprises the susceptibility. Alternatively, the reporting can comprise providing the at least one entity of the group with a login and password, which provides access to a report comprising the susceptibility posted on a password-protected computer system.

### Study Population

In a general sense, the methods and kits described herein can be utilized from samples containing nucleic acid material (DNA or RNA) or protein material from any source and from any individual, or from genotype or sequence data derived from such samples. In preferred embodiments, the individual is a human individual. The individual can be an adult, child, or fetus. The nucleic acid or protein source may be any sample comprising nucleic acid or protein material, including biological samples, or a sample comprising nucleic acid or protein material derived therefrom. The present invention also provides for assessing markers in individuals who are members of a target population. Such a target population is in one embodiment a population or group of individuals at risk of developing Gout, based on other genetic factors, biomarkers, biophysical parameters, or lifestyle factors.

The Icelandic population is a Caucasian population of Northern European ancestry. A large number of studies reporting results of genetic linkage and association in the Icelandic population have been published in the last few years. Many of those studies show replication of variants, originally identified in the Icelandic population as being associating with a particular disease, in other populations (Sulem, P., et al. Nat Genet May 17, 2009 (Epub ahead of print); Rafnar, T., et al. Nat Genet. 41:221-7 (2009); Gretarsdottir, S., et al. Ann Neurol 64:402-9 (2008); Stacey, S. N., et al. Nat Genet. 40:1313-18 (2008); Gudbjartsson, D. F., et al. Nat Genet. 40:886-91 (2008); Styrkarsdottir, U., et al. N Engl J Med 358:2355-65 (2008); Thorgeirsson, T., et al. Nature 452:638-42 (2008); Gudmundsson, J., et al. Nat. Genet. 40:281-3 (2008); Stacey, S, N., et al., Nat. Genet. 39:865-69 (2007); Helgadottir, A., et al., Science 316:1491-93 (2007); Steinthorsdottir, V., et al., Nat. Genet. 39:770-75 (2007); Gudmundsson, J., et al., Nat. Genet. 39:631-37 (2007); Frayling, T M, Nature Reviews Genet. 8:657-662 (2007); Amundadottir, L. T., et al., Nat. Genet. 38:652-58 (2006); Grant, S. F., et al., Nat. Genet. 38:320-23 (2006)). Thus, genetic findings in the Icelandic population have in general been replicated in other populations, including populations from Africa and Asia.

It is thus believed that the markers described herein to be associated with risk of Gout will show similar association in other human populations. Particular embodiments comprising individual human populations are thus also contemplated and within the scope of the invention. Such embodiments relate to human subjects that are from one or more human population including, but not limited to, Caucasian populations, European populations, American populations, Eurasian populations, Asian populations, Central/South Asian populations, East Asian populations, and African populations. In certain embodiments, the invention pertains to individuals from Caucasian populations. In certain embodiments, the invention pertains to Icelandic individuals.

The racial contribution in individual subjects may also be determined by genetic analysis. Genetic analysis of ancestry may be carried out using unlinked microsatellite markers such as those set out in Smith et al. (Am J Hum Genet. 74, 1001-13 (2004)).

In certain embodiments, the invention relates to markers identified in specific populations, as described in the above. The person skilled in the art will appreciate that measures of linkage disequilibrium (LD) may give different results when applied to different populations. This is due to different population history of different human populations as well as differential selective pressures that may have led to differences in LD in specific genomic regions. It is also well known to the person skilled in the art that certain markers, e.g. SNP markers, have different population frequency in different populations, or are polymorphic in one population but not in another. The person skilled in the art will however apply the methods available and as taught herein to practice the present invention in any given human population. This may include assessment of polymorphic markers in the LD region of the present invention, so as to identify those markers that give strongest association within the specific population. Thus, the at-risk variants of the present invention may reside on different haplotype background and in different frequencies in various human populations. However, utilizing methods known in the art and the markers of the present invention, the invention can be practiced in any given human population.

### Screening Methods

The invention also provides a method of screening candidate markers for assessing susceptibility to Gout. The invention also provides a method of identification of a marker for use in assessing susceptibility to Gout. The method may comprise analyzing the frequency of at least one allele of a polymorphic marker in a population of human individuals diagnosed with Gout, wherein a significant difference in frequency of the at least one allele in the population of human individuals diagnosed with Gout as compared to the frequency of the at least one allele in a control population of human individuals is indicative of the allele as a marker of Gout. In certain embodiments, the candidate marker is a marker in linkage disequilibrium with SG19S921.

In one embodiment, the method comprises (i) identifying at least one polymorphic marker in linkage disequilibrium, as determined by values of r² of greater than 0.5, with SG19S921; (ii) obtaining sequence information about the at least one polymorphic marker in a group of individuals diagnosed with Gout; and (iii) obtaining sequence information about the at least one polymorphic marker in a group of control individuals; wherein determination of a significant difference in frequency of at least one allele in the at least one polymorphism in individuals diagnosed with Gout as compared with the frequency of the at least one allele in the control group is indicative of the at least one polymorphism being useful for assessing susceptibility to Gout.

In one embodiment, an increase in frequency of the at least one allele in the at least one polymorphism in individuals diagnosed with Gout, as compared with the frequency of the at least one allele in the control group, is indicative of the at least one polymorphism being useful for assessing increased susceptibility to Gout. In another embodiment, a decrease in frequency of the at least one allele in the at least one polymorphism in individuals diagnosed with Gout, as compared with the frequency of the at least one allele in the control group, is indicative of the at least one polymorphism being useful for assessing decreased susceptibility to, or protection against, Gout.

### Utility of Genetic Testing

The P527R mutation confers a high risk of Gout, increasing the lifetime risk of Gout from about 5% in non-carriers to about 30% for carriers. While identification of the mutation does not establish a diagnosis in itself, its identification has significant diagnostic implications given the high penetrance of the mutation.

Particularly based on the high risk associated with this variant, the variant may be clinically useful in identifying those individuals who have intermittent or vague symptoms who also have high risk of Gout and may thus benefit from a more thorough clinical evaluation.

Individuals identified thus identified as having symptoms consistent with an onset of Gout, could then be closely monitored or selected for immediate clinical work-up (including for example joint fluid examination) or selected for immediate therapy. Common therapy for Gout includes for example NSAIDs or colchicine for immediate alleviation, and uric acid lowering therapy for long-term treatment.

Alternatively, carriers of the risk variants for Gout and elevated uric acid levels described herein (e.g., P527R) could be recommended close monitoring of uric acid levels. If uric acid levels exceed recommended concentration in blood in carriers, preventive treatment could be initiated, for example by lifestyle changes or uric acid-lowering therapy. This way, onset of the disease could be prevented.

### Diagnostic Methods

Polymorphic markers associated with increased susceptibility of Gout are useful in diagnostic methods. While methods of diagnosing Gout are known in the art, the detection risk markers for Gout advantageously may be useful for detection of Gout at its early stages and may also reduce the occurrence of misdiagnosis. In this regard, the invention further provides methods of diagnosing Gout comprising obtaining sequence data identifying at least one risk allele as described herein, in conjunction with carrying out one or more clinical diagnostic steps, such as any of those described herein.

The present invention pertains in some embodiments to methods of clinical applications of diagnosis, e.g., diagnosis performed by a medical professional. In other embodiments, the invention pertains to methods of diagnosis or methods of determination of a susceptibility performed by a layman. The layman can be the customer of a sequencing or genotyping service. The layman may also be a genotype or sequencing service provider, who performs analysis on a DNA sample from an individual, in order to provide service related to genetic risk factors for particular traits or diseases, based on the genotype status of the individual (i.e., the customer). Sequencing methods include for example those discussed in the above, but in general any suitable sequencing method may be used in the methods described and claimed herein. Recent technological advances in genotyping technologies, including high-throughput genotyping of SNP markers, such as Molecular Inversion Probe array technology (e.g., Affymetrix GeneChip), and BeadArray Technologies (e.g., Illumina GoldenGate and Infinium assays) have made it possible for individuals to have their own genome assessed for up to one million SNPs simultaneously, at relatively little cost. The resulting genotype information, which can be made available to the individual, can be compared to information about disease or trait risk associated with various SNPs, including information from public literature and scientific publications.

The application of disease-associated alleles as described herein, can thus for example be performed by the individual, through analysis of his/her genotype data, by a health professional based on results of a clinical test, or by a third party, including the genotype or sequencing service provider. The third party may also be service provider who interprets genotype or sequence information from the customer to provide service related to specific genetic risk factors, including the genetic markers described herein. In other words, the diagnosis or determination of a susceptibility of genetic risk can be made by health professionals, genetic counselors, third parties providing genotyping and/or sequencing service, third parties providing risk assessment service or by the layman (e.g., the individual), based on information about the genotype status of an individual and knowledge about the risk conferred by particular genetic risk factors (e.g., particular SNPs). In the present context, the term “diagnosing”, “diagnose a susceptibility” and “determine a susceptibility” is meant to refer to any available method for determining a susceptibility or risk of disease, including those mentioned above.

In certain embodiments, a sample containing genomic DNA or protein from an individual is collected. Such sample can for example be a buccal swab, a saliva sample, a blood sample, or other suitable samples containing genomic DNA or protein, as described further herein. In certain embodiments, the sample is obtained by non-invasive means (e.g., for obtaining a buccal sample, saliva sample, hair sample or skin sample). In certain embodiments, the sample is obtained by non-surgical means, i.e. in the absence of a surgical intervention on the individual that puts the individual at substantial health risk. Such embodiments may, in addition to non-invasive means also include obtaining sample by extracting a blood sample (e.g., a venous blood sample). The genomic DNA or protein obtained from the individual is then analyzed using any common technique available to the skilled person, such as high-throughput technologies for genotyping and/or sequencing. Results from such methods are stored in a convenient data storage unit, such as a data carrier, including computer databases, data storage disks, or by other convenient data storage means. In certain embodiments, the computer database is an object database, a relational database or a post-relational database. The genotype data is subsequently analyzed for the presence of certain variants known to be susceptibility variants for a particular human condition, such as the genetic variants described herein associated with risk of Gout. Genotype and/or sequencing data can be retrieved from the data storage unit using any convenient data query method. Calculating risk conferred by a particular genotype for the individual can be based on comparing the genotype of the individual to previously determined risk (expressed as a relative risk (RR) or and odds ratio (OR), for example) for the genotype, for example for a heterozygous carrier of an at-risk variant. The calculated risk for the individual can be the relative risk for a person, or for a specific genotype of a person, compared to the average population with matched gender and ethnicity. The average population risk can be expressed as a weighted average of the risks of different genotypes, using results from a reference population, and the appropriate calculations to calculate the risk of a genotype group relative to the population can then be performed. Alternatively, the risk for an individual is based on a comparison of particular genotypes, for example heterozygous carriers of an at-risk allele of a marker compared with non-carriers of the at-risk allele. The calculated risk estimated can be made available to the customer via a website, preferably a secure website.

### Prognostic Methods

In addition to the utilities described above, the polymorphic markers of the invention are useful in determining a prognosis of a human individual experiencing symptoms associated with, or an individual diagnosed with Gout. Accordingly, the invention provides a method of predicting prognosis of an individual experiencing symptoms associated with, or an individual diagnosed with, Gout. The method comprises analyzing sequence data about a human individual for at least one polymorphic marker in the human ALDH16A1 gene, wherein different alleles of the at least one polymorphic marker are associated with different susceptibilities to Gout in humans, and predicting prognosis of the individual from the sequence data. In one embodiment, the at least one polymorphic marker selected from the group consisting of SG19S921, and markers in linkage disequilibrium therewith. In one embodiment, the polymorphic marker is the P527R substitution in an encoded ALDH16A1 protein.

The prognosis predicted by the methods of the invention can be any type of prognosis relating to the progression of Gout, and/or relating to the chance of recovering from Gout. The prognosis can, for instance, relate to the severity of Gout, or how the condition will respond to therapeutic treatment.

With regard to the prognostic methods described herein, the sequence data can be nucleic acid sequence data or amino acid sequence data. For example, in one embodiment, determination of the presence of the P527R substitution in a protein with sequence as set forth in SEQ ID NO:3 herein is indicative of risk of Gout, and may thus also be useful in prognostic applications. Suitable methods of obtaining each are known in the art, some of which are described herein.

### Methods for Predicting Response to Therapeutic Agents

As is known in the art, individuals can have differential responses to a particular therapy (e.g., a therapeutic agent or therapeutic method). Pharmacogenomics addresses the issue of how genetic variations (e.g., the variants (markers and/or haplotypes) of the invention) affect drug response, due to altered drug disposition and/or abnormal or altered action of the drug. Thus, the basis of the differential response may be genetically determined in part. Clinical outcomes due to genetic variations affecting drug response may result in toxicity of the drug in certain individuals (e.g., carriers or non-carriers of the genetic variants of the invention), or therapeutic failure of the drug.

The variants described herein are predictive of uric acid levels in humans and risk of gout. Therapeutic intervention for gout includes agents that adjust uric acid concentration, and the therapeutic effect of such agents may in part be modulated by these variants. It is thus postulated that the variants of the invention may determine the manner in which a therapeutic agent for gout acts on the body, or the way in which the body metabolizes the therapeutic agent.

Treatment of acute attacks of Gout includes rest, application of ice to the affected joint, and prescription of colchicine (N-[(75)-1,2,3,10-tetramethoxy-9-oxo-5,6,7,9-tetrahydrobenzo[a]heptalen-7-yl]acetamide), non-steroidal anti-inflammatory drugs, or both. Once an attack has subsided, levels of uric acid are reduced by lifestyle changes, and for those individuals who experience recurrent attacks, uric acid lowering agents provide long-term prevention by lowering uric acid levels. The aim of urate-lowering therapy is to maintain urate concentration below the saturation point for monosdium urate. Current guidelines recommend that plasma urate should be maintained at a concentration less than 360 μmol/l (EULAR) or 300 μmol (British guidelines) (Richette, P. & Bardin, T., Lancet 2010; 375:318-328).

Uric acid-lowering agents include allopurinol (3,5,7,8-tetrazabicyclo[4.3.0]nona-3,5,9-trien-2-one), which is a first-line treatment option, uricosuric agents such as probenecid (4-(dipropylsulfamoyl)benzoic acid), pegloticase (recombinant porcine-like uricase), and sulfinpyrazone (1,2-diphenyl-4-[2-(phenylsulfinyl)ethyl]pyrazolidine-3,5-dione), benzbromarone ((3,5-dibromo-4-hydroxyphenyl)-(2-ethyl-3-benzofuranyl)methanone), and the xanthine oxidase inhibitor febuxostat (2-(3-cyano-4-isobutoxyphenyl)-4-methyl-1,3-thiazole-5-carboxylic acid)

Accordingly, in one embodiment, the presence of a particular allele at a polymorphic site (e.g., a G allele at SG19S921 and/or the P527R substitution in ALDH16A1) is indicative of a different response, e.g. a different response rate, to a particular treatment modality for Gout. This means that a patient diagnosed with Gout, and carrying such risk alleles would respond better to, or worse to, a specific therapeutic, drug and/or other therapy used to treat the condition. Therefore, the presence or absence of the marker allele could aid in deciding what treatment should be used for the patient. If the patient is positive for the marker allele, then the physician recommends one particular therapy, while if the patient is negative for the at least one allele of a marker, then a different course of therapy may be recommended (which may include recommending that no immediate therapy, other than serial monitoring for progression of symptoms, be performed). Thus, the patient's carrier status could be used to help determine whether a particular treatment modality should be administered. The treatment modality may be any of the treatment options mentioned in the foregoing. In one embodiment, determination of the presence of at least one copy of the G allele of marker SG19S921 and/or the P527R substitution in ALDH16A1 is indicative of a positive response to the therapeutic agent. In certain embodiments, the therapeutic agent is a uric acid-lowering agent.

Another aspect of the invention relates to methods of selecting individuals suitable for a particular treatment modality, based on their likelihood of developing particular complications or side effects of the particular treatment. It is well known that many therapeutic agents can lead to certain unwanted complications or side effects. Likewise, certain therapeutic procedures or operations may have complications associated with them. Complications or side effects of these particular treatments or associated with specific therapeutic agents can, just as diseases do, have a genetic component. It is therefore contemplated that selection of the appropriate treatment or therapeutic agent can in part be performed by determining the genotype of an individual, and using the genotype status (e.g., the presence or absence of the SG19S921 G allele or the P527R substitution in ALDH16A1) of the individual to decide on a suitable therapeutic procedure or on a suitable therapeutic agent to treat Gout. It is therefore contemplated that the polymorphic markers of the invention can be used in this manner. Indiscriminate use of a such therapeutic agents or treatment modalities may lead to unnecessary and needless adverse complications.

In view of the foregoing, the invention provides a method of assessing an individual for probability of response to a therapeutic agent for preventing, treating, and/or ameliorating symptoms associated Gout. In one embodiment, the method comprises: analyzing nucleic acid sequence data from a human individual for at least one polymorphic marker in the human ALDH16A1 gene, or an encoded ALDH16A1 protein, wherein different alleles of the at least one marker are indicative of different susceptibilities to gout in humans, and wherein a determination of the presence of the at least one allele is indicative of a probability of a positive response to the therapeutic agent. In certain embodiments, the at least one marker is selected from the group consisting of SG19S921, and markers in linkage disequilibrium therewith. In certain embodiments, the at least one encoded ALDH16A1 variant is the P527R substitution.

In a further aspect, the markers of the invention can be used to increase power and effectiveness of clinical trials. Thus, individuals who are carriers of the P527R variant in ALDH16A1 protein, or SG19S921 G variant in the ALDH16A1 gene, or other variants in the ALDH16A1 gene that confer risk of Gout, may be more likely to respond to a particular treatment modality. For some treatments, the genetic risk may correlate with less responsiveness to therapy. This application can improve the safety of clinical trials, but can also enhance the chance that a clinical trial will demonstrate statistically significant efficacy, which may be limited to a certain sub-group of the population. Thus, one possible outcome of such a trial is that carriers of the at-risk markers of the invention are statistically significantly likely to show positive response to the therapeutic agent, i.e. experience alleviation of symptoms associated with Gout, when taking the therapeutic agent or drug as prescribed. Another possible outcome is that genetic carriers show less favorable response to the therapeutic agent, or show differential side-effects to the therapeutic agent compared to the non-carrier. An aspect of the invention is directed to screening for such pharmacogenetic correlations.

### Kits

Kits useful in the methods of the invention comprise components useful in any of the methods described herein, including for example, primers for nucleic acid amplification, hybridization probes, restriction enzymes (e.g., for RFLP analysis), allele-specific oligonucleotides, antibodies, e.g., antibodies that bind to an altered ALDH16A1 polypeptide (e.g. the P527R variant) or to a non-altered (native) ALDH16A1 polypeptide, means for amplification of nucleic acids, means for analyzing the nucleic acid sequence of nucleic acids, means for analyzing the amino acid sequence of polynucleotides, etc. The kits can for example include necessary buffers, nucleic acid primers for amplifying nucleic acids (e.g., a nucleic acid segment comprising one or more of the polymorphic markers as described herein), and reagents for allele-specific detection of the fragments amplified using such primers and necessary enzymes (e.g., DNA polymerase). Additionally, kits can provide reagents for assays to be used in combination with the methods of the present invention, e.g., reagents for use with other diagnostic assays for Gout or related conditions.

In one embodiment, the invention pertains to a kit for assaying a sample from a subject to detect a susceptibility to Gout in the subject, wherein the kit comprises reagents necessary for selectively detecting at least one at-risk variant for Gout in the individual, wherein the at least one at-risk variant is a marker in the human ALDH16A1 gene or an amino acid substitution in an encoded ALDH16A1 protein. In a particular embodiment, the reagents comprise at least one contiguous oligonucleotide that hybridizes to a fragment of the genome of the individual comprising at least one polymorphism of the present invention. In another embodiment, the reagents comprise at least one pair of oligonucleotides that hybridize to opposite strands of a genomic segment obtained from a subject, wherein each oligonucleotide primer pair is designed to selectively amplify a fragment of the genome of the individual that includes at least one polymorphism associated with the condition risk. In one such embodiment, the polymorphism is selected from the group consisting of SG19S921, and polymorphic markers in linkage disequilibrium therewith. In yet another embodiment the fragment is at least 20 base pairs in size. Such oligonucleotides or nucleic acids (e.g., oligonucleotide primers) can be designed using portions of the nucleic acid sequence flanking the polymorphism. In another embodiment, the kit comprises one or more labeled nucleic acids capable of allele-specific detection of one or more specific polymorphic markers or haplotypes, and reagents for detection of the label. Suitable labels include, e.g., a radioisotope, a fluorescent label, an enzyme label, an enzyme co-factor label, a magnetic label, a spin label, an epitope label.

In one embodiment, the DNA template is amplified before detection by PCR. The DNA template may also be amplified by means of Whole Genome Amplification (WGA) methods, prior to assessment for the presence of specific polymorphic markers as described herein. Standard methods well known to the skilled person for performing WGA may be utilized, and are within scope of the invention. In one such embodiment, reagents for performing WGA are included in the reagent kit.

In certain embodiments, determination of the presence of a particular marker allele (e.g. allele G of SG19S921) is indicative of an increased susceptibility of Gout. In another embodiment, determination of the presence of a marker allele is indicative of prognosis of Gout. In another embodiment, the presence of the marker allele or haplotype is indicative of response to a therapeutic agent for Gout. In yet another embodiment, the presence of the marker allele or haplotype is indicative of progress of treatment of Gout.

In certain embodiments, the kit comprises reagents for detecting no more than 100 alleles in the genome of the individual. In certain other embodiments, the kit comprises reagents for detecting no more than 20 alleles in the genome of the individual.

In a further aspect of the present invention, a pharmaceutical pack (kit) is provided, the pack comprising a therapeutic agent and a set of instructions for administration of the therapeutic agent to humans diagnostically tested for an at-risk variant for Gout. The therapeutic agent can be a small molecule drug, an antibody, a peptide, an antisense or RNAi molecule, or other therapeutic molecules. In one embodiment, an individual identified as a carrier of at least one variant of the present invention is instructed to take a prescribed dose of the therapeutic agent. In one such embodiment, an individual identified as a homozygous carrier of at least one variant of the present invention (e.g., an at-risk variant) is instructed to take a prescribed dose of the therapeutic agent. In another embodiment, an individual identified as a non-carrier of at least one variant of the present invention (e.g., an at-risk variant) is instructed to take a prescribed dose of the therapeutic agent.

The kit may additionally or alternatively comprise reagents for detecting an amino acid substitution in a human ALDH16A1 protein, e.g., the P527R substitution and/or the P476R substitution. In one embodiment, the kit comprises at least one antibody for selectively detecting the P527R and/or the P476R substitution. Other reagents useful for detecting amino acid substitutions are known to the skilled person and are also contemplated.

In certain embodiments, the kit further comprises a set of instructions for using the reagents comprising the kit. In certain embodiments, the kit further comprises a collection of data comprising correlation data between the at least one at-risk variant and susceptibility to Gout.

### Antisense Agents

The nucleic acids and/or variants described herein, e.g. the SG19S921 variant, or nucleic acids comprising their complementary sequence, may be used as antisense constructs to control gene expression in cells, tissues or organs. The methodology associated with antisense techniques is well known to the skilled artisan, and is for example described and reviewed in Antisense Drug Technology: Principles, Strategies, and Applications, Crooke, ed., Marcel Dekker Inc., New York (2001). In general, antisense agents (antisense oligonucleotides) are comprised of single stranded oligonucleotides (RNA or DNA) that are capable of binding to a complimentary nucleotide segment. By binding the appropriate target sequence, an RNA-RNA, DNA-DNA or RNA-DNA duplex is formed. The antisense oligonucleotides are complementary to the sense or coding strand of a gene. It is also possible to form a triple helix, where the antisense oligonucleotide binds to duplex DNA.

Several classes of antisense oligonucleotide are known to those skilled in the art, including cleavers and blockers. The former bind to target RNA sites, activate intracellular nucleases (e.g., RnaseH or Rnase L), that cleave the target RNA. Blockers bind to target RNA, inhibit protein translation by steric hindrance of the ribosomes. Examples of blockers include nucleic acids, morpholino compounds, locked nucleic acids and methylphosphonates (Thompson, Drug Discovery Today, 7:912-917 (2002)). Antisense oligonucleotides are useful directly as therapeutic agents, and are also useful for determining and validating gene function, for example by gene knock-out or gene knock-down experiments. Antisense technology is further described in Layery et al., Curr. Opin. Drug Discov. Devel. 6:561-569 (2003), Stephens et al., Curr. Opin. Mol. Ther. 5:118-122 (2003), Kurreck, Eur. J. Biochem. 270:1628-44 (2003), Dias et al., Mol. Cancer. Ter. 1:347-55 (2002), Chen, Methods Mol. Med. 75:621-636 (2003), Wang et al., Curr. Cancer Drug Targets 1:177-96 (2001), and Bennett, Antisense Nucleic Acid Drug. Dev. 12:215-24 (2002).

In certain embodiments, the antisense agent is an oligonucleotide that is capable of binding to a particular nucleotide segment. In certain embodiments, the nucleotide segment comprises the human ALDH16A1 gene. In certain other embodiments, the antisense nucleotide is capable of binding to a nucleotide segment of the human ALDH16A1 gene, as set forth in SEQ ID NO:4. In one embodiment, the antisense nucleotide is capable of binding the a nucleotide segment of the human ALDH16A1 gene with sequence as set forth in SEQ ID NO:4 that has an C to G substitution in the polymorphic position of marker SG19S921 (position 12,534 in SEQ ID NO:4). Antisense nucleotides can be from 5-400 nucleotides in length, including 5-200 nucleotides, 5-100 nucleotides, 10-50 nucleotides, and 10-30 nucleotides. In certain preferred embodiments, the antisense nucleotides are from 14-50 nucleotides in length, including 14-40 nucleotides and 14-30 nucleotides.

The variants described herein can also be used for the selection and design of antisense reagents that are specific for particular variants. Using information about the variants described herein, antisense oligonucleotides or other antisense molecules that specifically target mRNA molecules that contain one or more variants of the invention can be designed. In this manner, expression of mRNA molecules that contain one or more variant of the present invention can be inhibited or blocked. In one embodiment, the antisense molecules are designed to specifically bind a particular allelic form of the target nucleic acid, thereby inhibiting translation of a product originating from this specific allele, but which do not bind other or alternate variants at the specific polymorphic sites of the target nucleic acid molecule. In one embodiment, the antisense molecule is designed to specifically bind to nucleic acids comprising the G allele of SG19S921. As antisense molecules can be used to inactivate mRNA so as to inhibit gene expression, and thus protein expression, the molecules can be used for disease treatment. The methodology can involve cleavage by means of ribozymes containing nucleotide sequences complementary to one or more regions in the mRNA that attenuate the ability of the mRNA to be translated. Such mRNA regions include, for example, protein-coding regions, in particular protein-coding regions corresponding to catalytic activity, substrate and/or ligand binding sites, or other functional domains of a protein.

The phenomenon of RNA interference (RNAi) has been actively studied for the last decade, since its original discovery in C. elegans (Fire et al., Nature 391:806-11 (1998)), and in recent years its potential use in treatment of human disease has been actively pursued (reviewed in Kim & Rossi, Nature Rev. Genet. 8:173-204 (2007)). RNA interference (RNAi), also called gene silencing, is based on using double-stranded RNA molecules (dsRNA) to turn off specific genes. In the cell, cytoplasmic double-stranded RNA molecules (dsRNA) are processed by cellular complexes into small interfering RNA (siRNA). The siRNA guide the targeting of a protein-RNA complex to specific sites on a target mRNA, leading to cleavage of the mRNA (Thompson, Drug Discovery Today, 7:912-917 (2002)). The siRNA molecules are typically about 20, 21, 22 or 23 nucleotides in length. Thus, one aspect of the invention relates to isolated nucleic acid molecules, and the use of those molecules for RNA interference, i.e. as small interfering RNA molecules (siRNA). In one embodiment, the isolated nucleic acid molecules are 18-26 nucleotides in length, preferably 19-25 nucleotides in length, more preferably 20-24 nucleotides in length, and more preferably 21, 22 or 23 nucleotides in length.

Another pathway for RNAi-mediated gene silencing originates in endogenously encoded primary microRNA (pri-miRNA) transcripts, which are processed in the cell to generate precursor miRNA (pre-miRNA). These miRNA molecules are exported from the nucleus to the cytoplasm, where they undergo processing to generate mature miRNA molecules (miRNA), which direct translational inhibition by recognizing target sites in the 3′ untranslated regions of mRNAs, and subsequent mRNA degradation by processing P-bodies (reviewed in Kim & Rossi, Nature Rev. Genet. 8:173-204 (2007)).

Clinical applications of RNAi include the incorporation of synthetic siRNA duplexes, which preferably are approximately 20-23 nucleotides in size, and preferably have 3′ overlaps of 2 nucleotides. Knockdown of gene expression is established by sequence-specific design for the target mRNA. Several commercial sites for optimal design and synthesis of such molecules are known to those skilled in the art.

Other applications provide longer siRNA molecules (typically 25-30 nucleotides in length, preferably about 27 nucleotides), as well as small hairpin RNAs (shRNAs; typically about 29 nucleotides in length). The latter are naturally expressed, as described in Amarzguioui et al. (FEBS Lett. 579:5974-81 (2005)). Chemically synthetic siRNAs and shRNAs are substrates for in vivo processing, and in some cases provide more potent gene-silencing than shorter designs (Kim et al., Nature Biotechnol. 23:222-226 (2005); Siolas et al., Nature Biotechnol. 23:227-231 (2005)). In general siRNAs provide for transient silencing of gene expression, because their intracellular concentration is diluted by subsequent cell divisions. By contrast, expressed shRNAs mediate long-term, stable knockdown of target transcripts, for as long as transcription of the shRNA takes place (Marques et al., Nature Biotechnol. 23:559-565 (2006); Brummelkamp et al., Science 296: 550-553 (2002)).

Since RNAi molecules, including siRNA, miRNA and shRNA, act in a sequence-dependent manner, the variants presented herein can be used to design RNAi reagents that recognize specific nucleic acid molecules comprising specific alleles and/or haplotypes (e.g., the alleles and/or haplotypes of the present invention), while not recognizing nucleic acid molecules comprising other alleles or haplotypes. These RNAi reagents can thus recognize and destroy the target nucleic acid molecules. As with antisense reagents, RNAi reagents can be useful as therapeutic agents (i.e., for turning off disease-associated genes or disease-associated gene variants), but may also be useful for characterizing and validating gene function (e.g., by gene knock-out or gene knock-down experiments).

Delivery of RNAi may be performed by a range of methodologies known to those skilled in the art. Methods utilizing non-viral delivery include cholesterol, stable nucleic acid-lipid particle (SNALP), heavy-chain antibody fragment (Fab), aptamers and nanoparticles. Viral delivery methods include use of lentivirus, adenovirus and adeno-associated virus. The siRNA molecules are in some embodiments chemically modified to increase their stability. This can include modifications at the 2′ position of the ribose, including 2′-O-methylpurines and 2′-fluoropyrimidines, which provide resistance to Rnase activity. Other chemical modifications are possible and known to those skilled in the art.

The following references provide a further summary of RNAi, and possibilities for targeting specific genes using RNAi: Kim & Rossi, Nat. Rev. Genet. 8:173-184 (2007), Chen & Rajewsky, Nat. Rev. Genet. 8: 93-103 (2007), Reynolds, et al., Nat. Biotechnol. 22:326-330 (2004), Chi et al., Proc. Natl. Acad. Sci. USA 100:6343-6346 (2003), Vickers et al., J. Biol. Chem. 278:7108-7118 (2003), Agami, Curr. Opin. Chem. Biol. 6:829-834 (2002), Layery, et al., Curr. Opin. Drug Discov. Devel. 6:561-569 (2003), Shi, Trends Genet. 19:9-12 (2003), Shuey et al., Drug Discov. Today 7:1040-46 (2002), McManus et al., Nat. Rev. Genet. 3:737-747 (2002), Xia et al., Nat. Biotechnol. 20:1006-10 (2002), Plasterk et al., curr. Opin. Genet. Dev. 10:562-7 (2000), Bosher et al., Nat. Cell Biol. 2:E31-6 (2000), and Hunter, Curr. Biol. 9:R440-442 (1999).

### Nucleic acids and polypeptides

The nucleic acids and polypeptides described herein can be used in methods and kits of the present invention. An “isolated” nucleic acid molecule, as used herein, is one that is separated from nucleic acids that normally flank the gene or nucleotide sequence (as in genomic sequences) and/or has been completely or partially purified from other transcribed sequences (e.g., as in an RNA library). for example, an isolated nucleic acid of the invention can be substantially isolated with respect to the complex cellular milieu in which it naturally occurs, or culture medium when produced by recombinant techniques, or chemical precursors or other chemicals when chemically synthesized. In some instances, the isolated material will form part of a composition (for example, a crude extract containing other substances), buffer system or reagent mix. In other circumstances, the material can be purified to essential homogeneity, for example as determined by polyacrylamide gel electrophoresis (PAGE) or column chromatography (e.g., HPLC). An isolated nucleic acid molecule of the invention can comprise at least about 50%, at least about 80% or at least about 90% (on a molar basis) of all macromolecular species present. With regard to genomic DNA, the term “isolated” also can refer to nucleic acid molecules that are separated from the chromosome with which the genomic DNA is naturally associated. For example, the isolated nucleic acid molecule can contain less than about 250 kb, 200 kb, 150 kb, 100 kb, 75 kb, 50 kb, 25 kb, 10 kb, 5 kb, 4 kb, 3 kb, 2 kb, 1 kb, 0.5 kb or 0.1 kb of the nucleotides that flank the nucleic acid molecule in the genomic DNA of the cell from which the nucleic acid molecule is derived.

The invention also pertains to nucleic acid molecules that hybridize under high stringency hybridization conditions, such as for selective hybridization, to a nucleotide sequence described herein (e.g., nucleic acid molecules that specifically hybridize to a nucleotide sequence containing a polymorphic site associated with a marker or haplotype described herein). Such nucleic acid molecules can be detected and/or isolated by allele- or sequence-specific hybridization (e.g., under high stringency conditions). Stringency conditions and methods for nucleic acid hybridizations are well known to the skilled person (see, e.g., Current Protocols in Molecular Biology, Ausubel, F. et al, John Wiley & Sons, (1998), and Kraus, M. and Aaronson, S., Methods Enzymol., 200:546-556 (1991), the entire teachings of which are incorporated by reference herein.

The percent identity of two nucleotide or amino acid sequences can be determined by aligning the sequences for optimal comparison purposes (e.g., gaps can be introduced in the sequence of a first sequence). The nucleotides or amino acids at corresponding positions are then compared, and the percent identity between the two sequences is a function of the number of identical positions shared by the sequences (i.e., % identity=# of identical positions/total # of positions×100). In certain embodiments, the length of a sequence aligned for comparison purposes is at least 30%, at least 40%, at least 50%, at least 60%, at least 70%, at least 80%, at least 90%, or at least 95%, of the length of the reference sequence. The actual comparison of the two sequences can be accomplished by methods well known to the skilled person, for example, using the NBLAST and XBLAST programs, as described in Altschul, S. et al., Nucleic Acids Res., 25:3389-3402 (1997). Another example of an algorithm is BLAT (Kent, W. J. Genome Res. 12:656-64 (2002)).

The present invention also provides isolated nucleic acid molecules that contain a fragment or portion that hybridizes under highly stringent conditions to a nucleic acid that comprises, or consists of, the nucleotide sequence of the human ALDH16A1 gene as set forth in SEQ ID NO:4, or a nucleotide sequence comprising, or consisting of, the complement of the nucleotide sequence of SEQ ID NO:4, wherein the nucleotide sequence comprises at least one polymorphic allele contained in the markers described herein (e.g., SG195921 aka rs150414818). The nucleic acid fragments of the invention are at least about 15, at least about 18, 20, 23 or 25 nucleotides, and can be up to 30, 40, 50, 100, 200, 300 or 400 nucleotides in length.

The nucleic acid fragments of the invention are used as probes or primers in assays such as those described herein. “Probes” or “primers” are oligonucleotides that hybridize in a base-specific manner to a complementary strand of a nucleic acid molecule. In addition to DNA and RNA, such probes and primers include polypeptide nucleic acids (PNA), as described in Nielsen, P. et al., Science 254:1497-1500 (1991). A probe or primer comprises a region of nucleotide sequence that hybridizes to at least about 15, typically about 20-25, and in certain embodiments about 40, 50 or 75, consecutive nucleotides of a nucleic acid molecule. In one embodiment, the probe or primer comprises at least one allele of at least one polymorphic marker or at least one haplotype described herein, or the complement thereof. In particular embodiments, a probe or primer can comprise 100 or fewer nucleotides; for example, in certain embodiments from 6 to 50 nucleotides, or, for example, from 12 to 30 nucleotides. In other embodiments, the probe or primer is at least 70% identical, at least 80% identical, at least 85% identical, at least 90% identical, or at least 95% identical, to the contiguous nucleotide sequence or to the complement of the contiguous nucleotide sequence. In another embodiment, the probe or primer is capable of selectively hybridizing to the contiguous nucleotide sequence or to the complement of the contiguous nucleotide sequence. Often, the probe or primer further comprises a label, e.g., a radioisotope, a fluorescent label, an enzyme label, an enzyme co-factor label, a magnetic label, a spin label, an epitope label.

### Computer-Implemented Aspects

As understood by those of ordinary skill in the art, the methods and information described herein may be implemented, in all or in part, as computer executable instructions on known computer readable media. For example, the methods described herein may be implemented in hardware. Alternatively, the method may be implemented in software stored in, for example, one or more memories or other computer readable medium and implemented on one or more processors. As is known, the processors may be associated with one or more controllers, calculation units and/or other units of a computer system, or implanted in firmware as desired. If implemented in software, the routines may be stored in any computer readable memory such as in RAM, ROM, flash memory, a magnetic disk, a laser disk, or other storage medium, as is also known. Likewise, this software may be delivered to a computing device via any known delivery method including, for example, over a communication channel such as a telephone line, the Internet, a wireless connection, etc., or via a transportable medium, such as a computer readable disk, flash drive, etc.

More generally, and as understood by those of ordinary skill in the art, the various steps described above may be implemented as various blocks, operations, tools, modules and techniques which, in turn, may be implemented in hardware, firmware, software, or any combination of hardware, firmware, and/or software. When implemented in hardware, some or all of the blocks, operations, techniques, etc. may be implemented in, for example, a custom integrated circuit (IC), an application specific integrated circuit (ASIC), a field programmable logic array (FPGA), a programmable logic array (PLA), etc.

When implemented in software, the software may be stored in any known computer readable medium such as on a magnetic disk, an optical disk, or other storage medium, in a RAM or ROM or flash memory of a computer, processor, hard disk drive, optical disk drive, tape drive, etc. Likewise, the software may be delivered to a user or a computing system via any known delivery method including, for example, on a computer readable disk or other transportable computer storage mechanism.

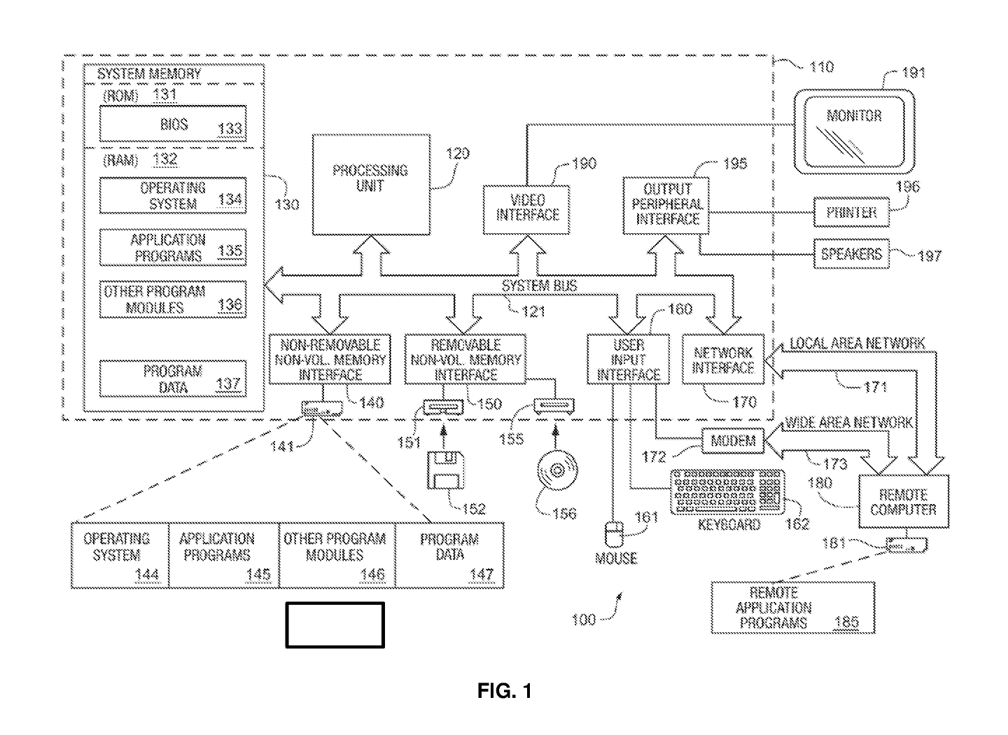

FIG. 1 illustrates an example of a suitable computing system environment 100 on which a system for the steps of the claimed method and apparatus may be implemented. The computing system environment 100 is only one example of a suitable computing environment and is not intended to suggest any limitation as to the scope of use or functionality of the method or apparatus of the claims. Neither should the computing environment 100 be interpreted as having any dependency or requirement relating to any one or combination of components illustrated in the exemplary operating environment 100.

The steps of the claimed method and system are operational with numerous other general purpose or special purpose computing system environments or configurations. Examples of well known computing systems, environments, and/or configurations that may be suitable for use with the methods or system of the claims include, but are not limited to, personal computers, server computers, hand-held or laptop devices, multiprocessor systems, microprocessor-based systems, set top boxes, programmable consumer electronics, network PCs, minicomputers, mainframe computers, distributed computing environments that include any of the above systems or devices, and the like.

The steps of the claimed method and system may be described in the general context of computer-executable instructions, such as program modules, being executed by a computer. Generally, program modules include routines, programs, objects, components, data structures, etc. that perform particular tasks or implement particular abstract data types. The methods and apparatus may also be practiced in distributed computing environments where tasks are performed by remote processing devices that are linked through a communications network. In both integrated and distributed computing environments, program modules may be located in both local and remote computer storage media including memory storage devices.

With reference to FIG. 1, an exemplary system for implementing the steps of the claimed method and system includes a general purpose computing device in the form of a computer 110. Components of computer 110 may include, but are not limited to, a processing unit 120, a system memory 130, and a system bus 121 that couples various system components including the system memory to the processing unit 120. The system bus 121 may be any of several types of bus structures including a memory bus or memory controller, a peripheral bus, and a local bus using any of a variety of bus architectures. By way of example, and not limitation, such architectures include Industry Standard Architecture (USA) bus, Micro Channel Architecture (MCA) bus, Enhanced ISA (EISA) bus, Video Electronics Standards Association (VESA) local bus, and Peripheral Component Interconnect (PCI) bus also known as Mezzanine bus.

Computer 110 typically includes a variety of computer readable media. Computer readable media can be any available media that can be accessed by computer 110 and includes both volatile and nonvolatile media, removable and non-removable media. By way of example, and not limitation, computer readable media may comprise computer storage media and communication media. Computer storage media includes both volatile and nonvolatile, removable and non-removable media implemented in any method or technology for storage of information such as computer readable instructions, data structures, program modules or other data. Computer storage media includes, but is not limited to, RAM, ROM, EEPROM, flash memory or other memory technology, CD-ROM, digital versatile disks (DVD) or other optical disk storage, magnetic cassettes, magnetic tape, magnetic disk storage or other magnetic storage devices, or any other medium which can be used to store the desired information and which can accessed by computer 110. Communication media typically embodies computer readable instructions, data structures, program modules or other data in a modulated data signal such as a carrier wave or other transport mechanism and includes any information delivery media. The term “modulated data signal” means a signal that has one or more of its characteristics set or changed in such a manner as to encode information in the signal. By way of example, and not limitation, communication media includes wired media such as a wired network or direct-wired connection, and wireless media such as acoustic, RF, infrared and other wireless media. Combinations of the any of the above should also be included within the scope of computer readable media.

The system memory 130 includes computer storage media in the form of volatile and/or nonvolatile memory such as read only memory (ROM) 131 and random access memory (RAM) 132. A basic input/output system 133 (BIOS), containing the basic routines that help to transfer information between elements within computer 110, such as during start-up, is typically stored in ROM 131. RAM 132 typically contains data and/or program modules that are immediately accessible to and/or presently being operated on by processing unit 120. By way of example, and not limitation, FIG. 1 illustrates operating system 134, application programs 135, other program modules 136, and program data 137.

The computer 110 may also include other removable/non-removable, volatile/nonvolatile computer storage media. By way of example only, FIG. 1 illustrates a hard disk drive 140 that reads from or writes to non-removable, nonvolatile magnetic media, a magnetic disk drive 151 that reads from or writes to a removable, nonvolatile magnetic disk 152, and an optical disk drive 155 that reads from or writes to a removable, nonvolatile optical disk 156 such as a CD ROM or other optical media. Other removable/non-removable, volatile/nonvolatile computer storage media that can be used in the exemplary operating environment include, but are not limited to, magnetic tape cassettes, flash memory cards, digital versatile disks, digital video tape, solid state RAM, solid state ROM, and the like. The hard disk drive 141 is typically connected to the system bus 121 through a non-removable memory interface such as interface 140, and magnetic disk drive 151 and optical disk drive 155 are typically connected to the system bus 121 by a removable memory interface, such as interface 150.

The drives and their associated computer storage media discussed above and illustrated in FIG. 1, provide storage of computer readable instructions, data structures, program modules and other data for the computer 110. In FIG. 1, for example, hard disk drive 141 is illustrated as storing operating system 144, application programs 145, other program modules 146, and program data 147. Note that these components can either be the same as or different from operating system 134, application programs 135, other program modules 136, and program data 137. Operating system 144, application programs 145, other program modules 146, and program data 147 are given different numbers here to illustrate that, at a minimum, they are different copies. A user may enter commands and information into the computer 20 through input devices such as a keyboard 162 and pointing device 161, commonly referred to as a mouse, trackball or touch pad. Other input devices (not shown) may include a microphone, joystick, game pad, satellite dish, scanner, or the like. These and other input devices are often connected to the processing unit 120 through a user input interface 160 that is coupled to the system bus, but may be connected by other interface and bus structures, such as a parallel port, game port or a universal serial bus (USB). A monitor 191 or other type of display device is also connected to the system bus 121 via an interface, such as a video interface 190. In addition to the monitor, computers may also include other peripheral output devices such as speakers 197 and printer 196, which may be connected through an output peripheral interface 190.

The computer 110 may operate in a networked environment using logical connections to one or more remote computers, such as a remote computer 180. The remote computer 180 may be a personal computer, a server, a router, a network PC, a peer device or other common network node, and typically includes many or all of the elements described above relative to the computer 110, although only a memory storage device 181 has been illustrated in FIG. 1. The logical connections depicted in FIG. 1 include a local area network (LAN) 171 and a wide area network (WAN) 173, but may also include other networks. Such networking environments are commonplace in offices, enterprise-wide computer networks, intranets and the Internet.

When used in a LAN networking environment, the computer 110 is connected to the LAN 171 through a network interface or adapter 170. When used in a WAN networking environment, the computer 110 typically includes a modem 172 or other means for establishing communications over the WAN 173, such as the Internet. The modem 172, which may be internal or external, may be connected to the system bus 121 via the user input interface 160, or other appropriate mechanism. In a networked environment, program modules depicted relative to the computer 110, or portions thereof, may be stored in the remote memory storage device. By way of example, and not limitation, FIG. 1 illustrates remote application programs 185 as residing on memory device 181. It will be appreciated that the network connections shown are exemplary and other means of establishing a communications link between the computers may be used.

While the risk evaluation system and method, and other elements, have been described as preferably being implemented in software, they may be implemented in hardware, firmware, etc., and may be implemented by any other processor. Thus, the elements described herein may be implemented in a standard multi-purpose CPU or on specifically designed hardware or firmware such as an application-specific integrated circuit (ASIC) or other hard-wired device as desired, including, but not limited to, the computer 110 of FIG. 1. When implemented in software, the software routine may be stored in any computer readable memory such as on a magnetic disk, a laser disk, or other storage medium, in a RAM or ROM of a computer or processor, in any database, etc. Likewise, this software may be delivered to a user or a diagnostic system via any known or desired delivery method including, for example, on a computer readable disk or other transportable computer storage mechanism or over a communication channel such as a telephone line, the internet, wireless communication, etc. (which are viewed as being the same as or interchangeable with providing such software via a transportable storage medium).

Thus, many modifications and variations may be made in the techniques and structures described and illustrated herein without departing from the spirit and scope of the present invention. Thus, it should be understood that the methods and apparatus described herein are illustrative only and are not limiting upon the scope of the invention.

Accordingly, certain aspects of the invention relate to computer-implemented applications using the polymorphic markers and haplotypes described herein, and genotype and/or disease-association data derived therefrom. Such applications can be useful for storing, manipulating or otherwise analyzing genotype data that is useful in the methods of the invention. One example pertains to storing genotype and/or sequence data derived from an individual on readable media, so as to be able to provide the data to a third party (e.g., the individual, a guardian of the individual, a health care provider or genetic analysis service provider), or for deriving information from the data, e.g., by comparing the data to information about genetic risk factors contributing to increased susceptibility to Gout, and reporting results based on such comparison.

In certain embodiments, computer-readable media suitably comprise capabilities of storing (i) identifier information for at least one polymorphic marker (e.g., marker names), as described herein; (ii) an indicator of the identity (e.g., presence or absence) of at least one allele of said at least one marker in individuals with Gout (e.g., SG19S921); and (iii) an indicator of the risk associated with a particular marker allele (e.g., the G allele of SG19S921) or a particular genotype (e.g., a C/G genotype at marker SG19S921). The media may also suitably comprise capabilities of storing protein sequence data.

In one embodiment, the invention provides a computer-readable medium having computer executable instructions for determining susceptibility to Gout in a human individual, the computer readable medium comprising (i) sequence data identifying at least one allele of at least one polymorphic marker in the individual; and (ii) a routine stored on the computer readable medium and adapted to be executed by a processor to determine risk of developing Gout for the at least one polymorphic marker; wherein the at least one polymorphic marker is a marker in the human ALDH16A1 gene, or an amino acid substitution in an encoded ALDH16A1 protein, that is predictive of susceptibility of Gout in humans. In one embodiment, the at least one polymorphic marker is SG19S921. In another embodiment, the amino acid substitution is the P527R substitution in the human ALDH16A1 polypeptide with sequence as set forth in SEQ ID NO:3.

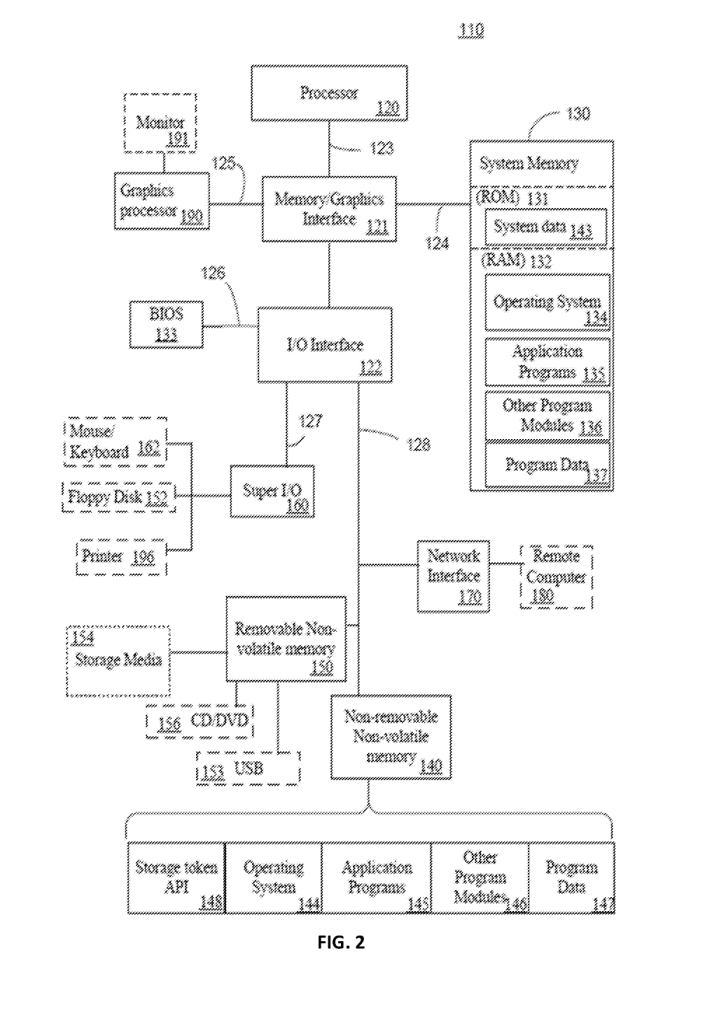

With reference to FIG. 2, a second exemplary system of the invention, which may be used to implement one or more steps of methods of the invention, includes a computing device in the form of a computer 110. Components shown in dashed outline are not technically part of the computer 110, but are used to illustrate the exemplary embodiment of FIG. 2. Components of computer 110 may include, but are not limited to, a processor 120, a system memory 130, a memory/graphics interface 121, also known as a Northbridge chip, and an I/O interface 122, also known as a Southbridge chip. The system memory 130 and a graphics processor 190 may be coupled to the memory/graphics interface 121. A monitor 191 or other graphic output device may be coupled to the graphics processor 190.

A series of system busses may couple various system components including a high speed system bus 123 between the processor 120, the memory/graphics interface 121 and the I/O interface 122, a front-side bus 124 between the memory/graphics interface 121 and the system memory 130, and an advanced graphics processing (AGP) bus 125 between the memory/graphics interface 121 and the graphics processor 190. The system bus 123 may be any of several types of bus structures including, by way of example, and not limitation, such architectures include Industry Standard Architecture (USA) bus, Micro Channel Architecture (MCA) bus and Enhanced ISA (EISA) bus. As system architectures evolve, other bus architectures and chip sets may be used but often generally follow this pattern. For example, companies such as Intel and AMD support the Intel Hub Architecture (IHA) and the Hypertransport™ architecture, respectively.

The computer 110 typically includes a variety of computer-readable media. Computer-readable media can be any available media that can be accessed by computer 110 and includes both volatile and nonvolatile media, removable and non-removable media. By way of example, and not limitation, computer readable media may comprise computer storage media. Computer storage media includes both volatile and nonvolatile, removable and non-removable media implemented in any method or technology for storage of information such as computer readable instructions, data structures, program modules or other data. Computer storage media includes, but is not limited to, RAM, ROM, EEPROM, flash memory or other memory technology, CD-ROM, digital versatile disks (DVD) or other optical disk storage, magnetic cassettes, magnetic tape, magnetic disk storage or other magnetic storage devices, or any other physical medium which can be used to store the desired information and which can accessed by computer 110.

The system memory 130 includes computer storage media in the form of volatile and/or nonvolatile memory such as read only memory (ROM) 131 and random access memory (RAM) 132. The system ROM 131 may contain permanent system data 143, such as identifying and manufacturing information. In some embodiments, a basic input/output system (BIOS) may also be stored in system ROM 131. RAM 132 typically contains data and/or program modules that are immediately accessible to and/or presently being operated on by processor 120. By way of example, and not limitation, FIG. 1 illustrates operating system 134, application programs 135, other program modules 136, and program data 137.

The I/O interface 122 may couple the system bus 123 with a number of other busses 126, 127 and 128 that couple a variety of internal and external devices to the computer 110. A serial peripheral interface (SPI) bus 126 may connect to a basic input/output system (BIOS) memory 133 containing the basic routines that help to transfer information between elements within computer 110, such as during start-up.

A super input/output chip 160 may be used to connect to a number of ‘legacy’ peripherals, such as floppy disk 152, keyboard/mouse 162, and printer 196, as examples. The super I/O chip 160 may be connected to the I/O interface 122 with a bus 127, such as a low pin count (LPC) bus, in some embodiments. Various embodiments of the super I/O chip 160 are widely available in the commercial marketplace.

In one embodiment, bus 128 may be a Peripheral Component Interconnect (PCI) bus, or a variation thereof, may be used to connect higher speed peripherals to the I/O interface 122. A PCI bus may also be known as a Mezzanine bus. Variations of the PCI bus include the Peripheral Component Interconnect-Express (PCI-E) and the Peripheral Component Interconnect-Extended (PCI-X) busses, the former having a serial interface and the latter being a backward compatible parallel interface. In other embodiments, bus 128 may be an advanced technology attachment (ATA) bus, in the form of a serial ATA bus (SATA) or parallel ATA (PATA).

The computer 110 may also include other removable/non-removable, volatile/nonvolatile computer storage media. By way of example only, FIG. 2 illustrates a hard disk drive 140 that reads from or writes to non-removable, nonvolatile magnetic media. The hard disk drive 140 may be a conventional hard disk drive.

Removable media, such as a universal serial bus (USB) memory 153, firewire (IEEE 1394), or CD/DVD drive 156 may be connected to the PCI bus 128 directly or through an interface 150. A storage media 154 may be coupled through interface 150. Other removable/non-removable, volatile/nonvolatile computer storage media that can be used in the exemplary operating environment include, but are not limited to, magnetic tape cassettes, flash memory cards, digital versatile disks, digital video tape, solid state RAM, solid state ROM, and the like.

The drives and their associated computer storage media discussed above and illustrated in FIG. 2, provide storage of computer readable instructions, data structures, program modules and other data for the computer 110. In FIG. 2, for example, hard disk drive 140 is illustrated as storing operating system 144, application programs 145, other program modules 146, and program data 147. Note that these components can either be the same as or different from operating system 134, application programs 135, other program modules 136, and program data 137. Operating system 144, application programs 145, other program modules 146, and program data 147 are given different numbers here to illustrate that, at a minimum, they are different copies. A user may enter commands and information into the computer 20 through input devices such as a mouse/keyboard 162 or other input device combination. Other input devices (not shown) may include a microphone, joystick, game pad, satellite dish, scanner, or the like. These and other input devices are often connected to the processor 120 through one of the I/O interface busses, such as the SPI 126, the LPC 127, or the PCI 128, but other busses may be used. In some embodiments, other devices may be coupled to parallel ports, infrared interfaces, game ports, and the like (not depicted), via the super I/O chip 160.

The computer 110 may operate in a networked environment using logical connections to one or more remote computers, such as a remote computer 180 via a network interface controller (NIC) 170. The remote computer 180 may be a personal computer, a server, a router, a network PC, a peer device or other common network node, and typically includes many or all of the elements described above relative to the computer 110. The logical connection between the NIC 170 and the remote computer 180 depicted in FIG. 2 may include a local area network (LAN), a wide area network (WAN), or both, but may also include other networks. Such networking environments are commonplace in offices, enterprise-wide computer networks, intranets, and the Internet. The remote computer 180 may also represent a web server supporting interactive sessions with the computer 110, or in the specific case of location-based applications may be a location server or an application server.

In some embodiments, the network interface may use a modem (not depicted) when a broadband connection is not available or is not used. It will be appreciated that the network connection shown is exemplary and other means of establishing a communications link between the computers may be used.

In some variations, the invention is a system for identifying susceptibility to Gout in a human subject. For example, in one variation, the system includes tools for performing at least one step, preferably two or more steps, and in some aspects all steps of a method of the invention, where the tools are operably linked to each other. Operable linkage describes a linkage through which components can function with each other to perform their purpose.

In some variations, a system of the invention is a system for identifying susceptibility to gout in a human subject, the system comprising:
at least one processor;
at least one computer-readable medium;
a susceptibility database operatively coupled to a computer-readable medium of the system and containing population information correlating the presence or absence of one or more alleles of the human ALDH16A1 gene and susceptibility to gout in a population of humans;
a measurement tool that receives an input about the human subject and generates information from the input about the presence or absence of at least one mutant ALDH16A1 allele indicative of a ALDH16A1 defect in the human subject; and
an analysis tool that:
is operatively coupled to the susceptibility database and the measurement tool,
is stored on a computer-readable medium of the system,
is adapted to be executed on a processor of the system, to compare the information about the human subject with the population information in the susceptibility database and generate a conclusion with respect to susceptibility to gout for the human subject.

Exemplary processors (processing units) include all variety of microprocessors and other processing units used in computing devices. Exemplary computer-readable media are described above. When two or more components of the system involve a processor or a computer-readable medium, the system generally can be created where a single processor and/or computer readable medium is dedicated to a single component of the system; or where two or more functions share a single processor and/or share a single computer readable medium, such that the system contains as few as one processor and/or one computer readable medium. In some variations, it is advantageous to use multiple processors or media, for example, where it is convenient to have components of the system at different locations. For instance, some components of a system may be located at a testing laboratory dedicated to laboratory or data analysis, whereas other components, including components (optional) for supplying input information or obtaining an output communication, may be located at a medical treatment or counseling facility (e.g., doctor's office, health clinic, HMO, pharmacist, geneticist, hospital) and/or at the home or business of the human subject (patient) for whom the testing service is performed.

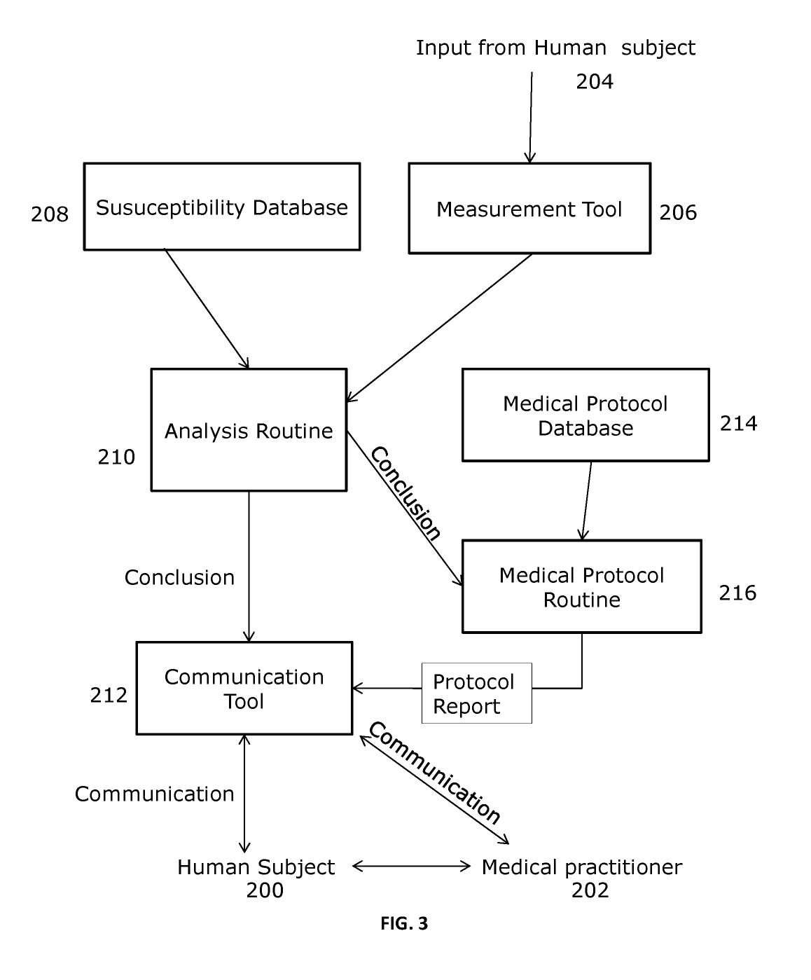

Referring to FIG. 3, an exemplary system includes a susceptibility database 208 that is operatively coupled to a computer-readable medium of the system and that contains population information correlating the presence or absence of one or more alleles of at least one ALDH16A1 mutant and susceptibility to Gout in a population of humans.

In a simple variation, the susceptibility database contains 208 data relating to the frequency that a particular marker allele selected from the group has been observed in a population of humans with Gout and a population of humans free of Gout. Such data provides an indication as to the relative risk or odds ratio of developing Gout for a human subject that is identified as having the allele in question. In another variation, the susceptibility database includes similar data with respect to two or more markers, thereby providing a useful reference if the human subject has any of the two or more alleles of the two or more markers. In still another variation, the susceptibility database includes additional quantitative personal, medical, or genetic information about the individuals in the database diagnosed with Gout or free of Gout. Such information includes, but is not limited to, information about parameters such as age, sex, ethnicity, race, medical history, weight, diabetes status, blood pressure, family history of Gout, smoking history, and alcohol use in humans and impact of the at least one parameter on susceptibility to Gout. The information also can include information about other genetic risk factors for gout besides the genetic variants described herein. These more robust susceptibility databases can be used by an analysis routine 210 to calculate a combined score with respect to susceptibility or risk for developing Gout.

In addition to the susceptibility database 208, the system further includes a measurement tool 206 programmed to receive an input 204 from or about the human subject and generate an output that contains information about the presence or absence of the at least one marker allele of interest. (The input 204 is not part of the system per se but is illustrated in the schematic FIG. 3.) Thus, the input 204 will contain a specimen or contain data from which the presence or absence of the at least one marker allele can be directly read, or analytically determined. In a simple variation, the input contains annotated information about genotypes or allele counts for particular markers such as SG19S921 or other mutant alleles in ALDH16A1 in the genome of the human subject, in which case no further processing by the measurement tool 206 is required, except possibly transformation of the relevant information about the presence/absence of the at least one marker allele into a format compatible for use by the analysis routine 210 of the system.

In another variation, the input 204 from the human subject contains data that is unannotated or insufficiently annotated with respect to risk markers for Gout e.g., mutant alleles in ALDH16A1, requiring analysis by the measurement tool 206. For example, the input can be genetic sequence of the chromosomal region or chromosome on which the markers reside, or whole genome sequence information, or unannotated information from a gene chip analysis of a variable loci in the human subject's genome. In such variations of the invention, the measurement tool 206 comprises a tool, preferably stored on a computer-readable medium of the system and adapted to be executed on a processor of the system, to receive a data input about a subject and determine information about the presence or absence of the at least one marker allele in a human subject from the data. For example, the measurement tool 206 contains instructions, preferably executable on a processor of the system, for analyzing the unannotated input data and determining the presence or absence of the marker allele of interest in the human subject. Where the input data is genomic sequence information, and the measurement tool optionally comprises a sequence analysis tool stored on a computer readable medium of the system and executable by a processor of the system with instructions for determining the presence or absence of the at least one mutant marker allele from the genomic sequence information.

In yet another variation, the input 204 from the human subject comprises a biological sample, such as a fluid (e.g., blood) or tissue sample that contains genetic material that can be analyzed to determine the presence or absence of particular marker allele(s) of interest. In this variation, an exemplary measurement tool 206 includes laboratory equipment for processing and analyzing the sample to determine the presence or absence (or identity) of the marker allele(s) in the human subject. For instance, in one variation, the measurement tool includes: an oligonucleotide microarray (e.g., “gene chip”) containing a plurality of oligonucleotide probes attached to a solid support; a detector for measuring interaction between nucleic acid obtained from or amplified from the biological sample and one or more oligonucleotides on the oligonucleotide microarray to generate detection data; and an analysis tool stored on a computer-readable medium of the system and adapted to be executed on a processor of the system, to determine the presence or absence of the at least one marker allele of interest based on the detection data.

To provide another example, in some variations the measurement tool 206 includes: a nucleotide sequencer (e.g., an automated DNA sequencer) that is capable of determining nucleotide sequence information from nucleic acid obtained from or amplified from the biological sample; and an analysis tool stored on a computer-readable medium of the system and adapted to be executed on a processor of the system, to determine the presence or absence of the at least one marker allele based on the nucleotide sequence information.

In some variations, the measurement tool 206 further includes additional equipment and/or chemical reagents for processing the biological sample to purify and/or amplify nucleic acid of the human subject for further analysis using a sequencer, gene chip, or other analytical equipment.

The exemplary system further includes an analysis tool or routine 210 that: is operatively coupled to the susceptibility database 208 and operatively coupled to the measurement tool 206, is stored on a computer-readable medium of the system, is adapted to be executed on a processor of the system to compare the information about the human subject with the population information in the susceptibility database 208 and generate a conclusion with respect to susceptibility to gout for the human subject. In simple terms, the analysis tool 210 looks at the marker alleles identified by the measurement tool 206 for the human subject, and compares this information to the susceptibility database 208, to determine a susceptibility to Gout for the subject. The susceptibility can be based on the single parameter (the identity of one or more marker alleles), or can involve a calculation based on other genetic and non-genetic data, as described above, that is collected and included as part of the input 204 from the human subject, and that also is stored in the susceptibility database 208 with respect to a population of other humans. Generally speaking, each parameter of interest is weighted to provide a conclusion with respect to susceptibility to Gout. Such a conclusion is expressed in the conclusion in any statistically useful form, for example, as an odds ratio, a relative risk, or a lifetime risk for subject developing Gout.

In some variations of the invention, the system as just described further includes a communication tool 212. For example, the communication tool is operatively connected to the analysis routine 210 and comprises a routine stored on a computer-readable medium of the system and adapted to be executed on a processor of the system, to: generate a communication containing the conclusion; and to transmit the communication to the human subject 200 or the medical practitioner 202, and/or enable the subject or medical practitioner to access the communication. (The subject and medical practitioner are depicted in the schematic FIG. 3, but are not part of the system per se, though they may be considered users of the system. The communication tool 212 provides an interface for communicating to the subject, or to a medical practitioner for the subject (e.g., doctor, nurse, genetic counselor), the conclusion generated by the analysis tool 210 with respect to susceptibility to Gout for the subject. Usually, if the communication is obtained by or delivered to the medical practitioner 202, the medical practitioner will share the communication with the human subject 200 and/or counsel the human subject about the medical significance of the communication. In some variations, the communication is provided in a tangible form, such as a printed report or report stored on a computer readable medium such as a flash drive or optical disk. In some variations, the communication is provided electronically with an output that is visible on a video display or audio output (e.g., speaker). In some variations, the communication is transmitted to the subject or the medical practitioner, e.g., electronically or through the mail. In some variations, the system is designed to permit the subject or medical practitioner to access the communication, e.g., by telephone or computer. For instance, the system may include software residing on a memory and executed by a processor of a computer used by the human subject or the medical practitioner, with which the subject or practitioner can access the communication, preferably securely, over the internet or other network connection. In some variations of the system, this computer will be located remotely from other components of the system, e.g., at a location of the human subject's or medical practitioner's choosing.

In some variations of the invention, the system as described (including embodiments with or without the communication tool) further includes components that add a treatment or prophylaxis utility to the system. For instance, value is added to a determination of susceptibility to gout when a medical practitioner can prescribe or administer a standard of care that can reduce susceptibility to Gout; and/or delay onset of Gout; and/or increase the likelihood of detecting gout at an early stage. Exemplary lifestyle change protocols include loss of weight, increase in exercise, cessation of unhealthy behaviors such as smoking, and change of diet. Exemplary medicinal and surgical intervention protocols include administration of pharmaceutical agents for prophylaxis; and surgery.

For example, in some variations, the system further includes a medical protocol database 214 operatively connected to a computer-readable medium of the system and containing information correlating the presence or absence of the at least one marker allele of interest and medical protocols for human subjects at risk for Gout. Such medical protocols include any variety of medicines, lifestyle changes, diagnostic tests, increased frequencies of diagnostic tests, and the like that are designed to achieve one of the aforementioned goals. The information correlating a marker allele with protocols could include, for example, information about the success with which gout is avoided or delayed, or success with which Gout is detected early and treated, if a subject has a particular susceptibility allele and follows a protocol.

The system of this embodiment further includes a medical protocol tool or routine 216, operatively connected to the medical protocol database 214 and to the analysis tool or routine 210. The medical protocol tool or routine 216 preferably is stored on a computer-readable medium of the system, and adapted to be executed on a processor of the system, to: (i) compare (or correlate) the conclusion that is obtained from the analysis routine 210 (with respect to susceptibility to Gout for the subject) and the medical protocol database 214, and (ii) generate a protocol report with respect to the probability that one or more medical protocols in the medical protocol database will achieve one or more of the goals of reducing susceptibility to gout; delaying onset of Gout; and increasing the likelihood of detecting gout at an early stage to facilitate early treatment. The probability can be based on empirical evidence collected from a population of humans and expressed either in absolute terms (e.g., compared to making no intervention), or expressed in relative terms, to highlight the comparative or additive benefits of two or more protocols.

Some variations of the system include the communication tool 212. In some examples, the communication tool generates a communication that includes the protocol report in addition to, or instead of, the conclusion with respect to susceptibility.

Information about marker allele status not only can provide useful information about identifying or quantifying susceptibility to Gout; it can also provide useful information about possible causative factors for a human subject identified with Gout, and useful information about therapies for the patient. In some variations, systems of the invention are useful for these purposes.

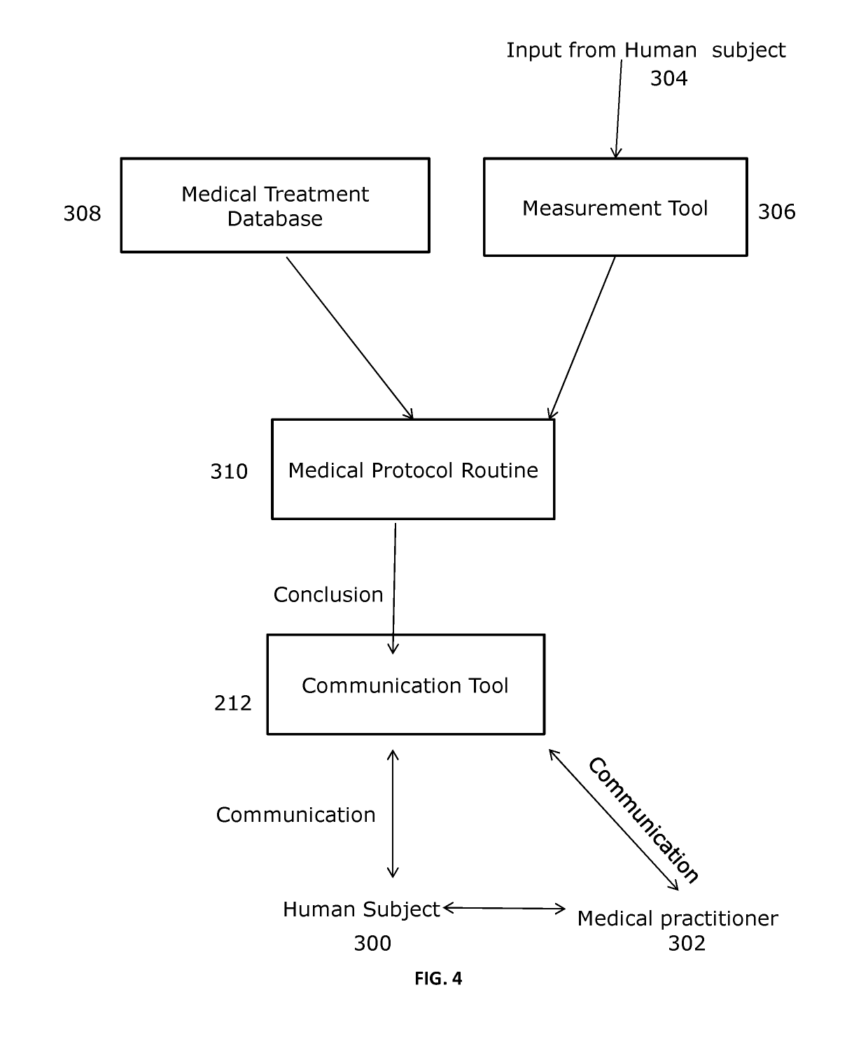

For instance, in some variations the invention is a system for assessing or selecting a treatment protocol for a subject diagnosed with Gout. An exemplary system, schematically depicted in FIG. 4, comprises:

at least one processor;

at least one computer-readable medium;

a medical treatment database operatively connected to a computer-readable medium of the system and containing information correlating the presence or absence of at least one mutant ALDH16A1 allele and efficacy of treatment regimens for gout;

a measurement tool to receive an input about the human subject and generate information from the input about the presence or absence of the at least one mutant ALDH16A1 allele indicative of a ALDH16A1 defect in a human subject diagnosed with gout; and

a medical protocol tool operatively coupled to the medical treatment database and the measurement tool, stored on a computer-readable medium of the system, and adapted to be executed on a processor of the system, to compare the information with respect to presence or absence of the at least one mutant ALDH16A1 allele for the subject and the medical treatment database, and generate a conclusion with respect to at least one of:
the probability that one or more medical treatments will be efficacious for treatment of gout for the patient; and
which of two or more medical treatments for gout will be more efficacious for the patient.

Preferably, such a system further includes a communication tool 312 operatively connected to the medical protocol tool or routine 310 for communicating the conclusion to the subject 300, or to a medical practitioner for the subject 302 (both depicted in the schematic of FIG. 4, but not part of the system per se). An exemplary communication tool comprises a routine stored on a computer-readable medium of the system and adapted to be executed on a processor of the system, to generate a communication containing the conclusion; and transmit the communication to the subject or the medical practitioner, or enable the subject or medical practitioner to access the communication.

In a further embodiment, the invention provides a computer-readable medium having computer executable instructions for determining susceptibility to Gout in a human individual, the computer readable medium comprising (i) sequence data identifying at least one allele of at least one polymorphic marker in the individual; and (ii) a routine stored on the computer readable medium and adapted to be executed by a processor to determine risk of developing Gout for the at least one polymorphic marker; wherein the at least one polymorphic marker is a marker in ALDH16A1 that is predictive of susceptibility of Gout in humans. In one embodiment, the at least one polymorphic marker is SG19S921, or a marker in linkage disequilibrium therewith. In one preferred embodiment, the polymorphic marker is SG19S921; also known as rs150414818.

In certain embodiments, a report is prepared, which contains results of a determination of susceptibility to Gout. The report may suitably be written in any computer readable medium, printed on paper, or displayed on a visual display.

The present invention will now be exemplified by the following non-limiting examples.

### Example 1

With the aim to search for sequence variants that predispose to Gout and uric acid levels, we performed a genome-wide association study (GWAS) on 766 Icelandic Gout cases and over 37,000 Icelandic population controls. All cases had reported daily use of anti-gout medication (ATC code M04 subgroup of the Anatomical Therapeutic Chemical Classification System developed by WHO).

Based on whole-genome sequencing of 292 Icelanders at 10-fold coverage, followed by imputation, we tested a total of 14 million single nucleotide polymorphisms (SNPs). Imputation was performed using one or more of four sources, the HapMap2 CEU sample (Nature 437, 1299-320 (2005)) (60 triads), the 1000 Genomes data (Durbin, R. M. et al. Nature 467, 1061-73) (179 individuals) and Icelandic samples genotyped with the Illumina Human1M-Duo and the HumanOmni1-Quad chips. Imputations were based on the IMPUTE model (Marchini, J., Howie, B., Myers, S., McVean, G. & Donnelly, P. Nat Genet. 39, 906-13 (2007)) and long range phasing of chip typed Icelandic samples (Kong, A. et al. Nat Genet (2008)).

The association analysis yielded a number of genome-wide significant association (P<5×10⁻⁸) signals between Gout on chromosome 19q13 (Table 1). The most significant SNP was found to be a novel C/G SNP marker at position 54,660,818 in NCBI Build 36 (SEQ ID NO:1), which has a frequency in the population of 1.9% and confers a risk of 4.26. This SNP marker, which is located in exon13 of the human ALDH16A1 gene, encodes a Proline to Arginine substitution in the encoded ALDH16A1 protein, at position 527 in the splice variant with sequence as shown in SEQ ID NO:3.

We also tested association to Uric Acid levels. As shown in Table 2, a number of variants on chromosome 19q13 were found to be genome-wide associated with Uric Acid levels. The most significant marker was the same one as in the association test with Uric Acid as a binary trait (P527R). Thus, variants on chromosome 19q13 both contribute to elevated Uric Acid levels in humans and risk of Gout.

Methods

The study was approved by the Data Protection Commission of Iceland and the National Bioethics Committee of Iceland. Written informed consent was obtained from all study participants. Personal identifiers associated with medical information and blood samples were encrypted with a third-party encryption system as provided by the Data Protection Commission of Iceland.

Icelandic Sample Sets.

Whole-Genome Sequencing. Sample Preparation:

Paired-end libraries for sequencing were prepared according to manufacturer's instructions (Illumina). In short, approximately 5 micrograms of genomic DNA, isolated from frozen blood samples, was fragmented to a mean target size of 300 basepairs (bp) using a Covaris E210 instrument. The resulting fragmented DNA was end-repaired using T4 and Klenow polymerases and T4 polynucleotide kinase with 10 mM dNTP's followed by addition of an “A” base at the ends using Klenow exo fragment (3′ to 5′-exo minus) and dATP (1 mM). Sequencing adaptors containing “T” overhangs were ligated to the DNA products followed by agarose (2%) gel electrophoresis. Fragments of about 400 bp were isolated from the gels (Qiagen Gel Extraction Kit) and the adaptor-modified DNA fragments were PCR enriched for 10-cycles using Phusion DNA polymerase (Finnzymes Oy) and PCR primers PE 1.0 and PE 2.0 (Illumina). Enriched libraries were further purified using agarose (2%) gel electrophoresis as described above. The quality and concentration of the libraries was assessed with the Agilent 2100 Bioanalyzer using the DNA 1000 LabChip (Agilent). Barcoded libraries were stored at −20° C. All steps in the workflow were monitored using an in-house laboratory information management system with barcode tracking of all samples and reagents. DNA sequencing: Template DNA fragments were hybridized to the surface of flow cells (Illumina PE flowcell, v4) and amplified to form clusters using the Illumina cBot. In brief, DNA (8-10 pM) was denatured followed by hybridization to grafted adaptors on the flowcell. Isothermal bridge amplification using Phusion polymerase was then followed by linearization of the bridged DNA, denaturation, blocking of 3′-ends and hybridization of the sequencing primer. Sequencing-by-synthesis was performed on Illumina GAIIx instruments equipped with paired-end modules. Paired-end libraries were sequenced using 2×101 cycles of incorporation and imaging with Illumina sequencing kits, v4. Each library/sample was initially run on a single lane for validation followed by further sequencing of lanes with targeted cluster densities of 250-300K/mm². Imaging and analysis of the data was performed using the SCS 2.6 and RTA 1.6 software packages from Illumina, respectively. RTA analysis involved conversion of image data to base-calling in real-time. Alignment: For each lane in the DNA sequencing output, the resulting qseq files were converted into fastq files using an inhouse script. All output from sequencing was converted and the Illumina quality filtering flag was retained in the output. The fastq files were then aligned against Build 36 of the human reference sequence using bwa version 0.5.7 (Li, H. & Durbin, R. Bioinformatics 25, 1754-60 (2009)). BAM file generation: SAM file output from the alignment was converted into BAM format using samtools version 0.1.8 (Li, H. et al. Bioinformatics 25, 2078-9 (2009)) and an inhouse script was used to carry the Illumina quality filter flag over to the BAM file. The BAM files for each sample were then merged into a single BAM file using samtools. Finally, Picard version 1.17 (see http://picard.sourceforge.net/) was used to mark duplicates in the resulting sample BAM files.

SNP Calling and Genotyping in Whole-Genome Sequencing.

A two step approach was applied to SNP genotyping the whole-genome sequencing data. First, a SNP detection step where sequence positions where at least one individual could be determined to be different from the reference sequence with confidence (quality threshold of 20) based on the SNP calling feature of the pileup tool of samtools (Li, H. et al. Bioinformatics 25, 2078-9 (2009)). SNPs that were always heterozygous, or always homozygous different from the reference were removed. Second, all positions that were flagged as polymorphic were then genotyped using the pileup tool, but since sequencing depth varies and hence the certainty of genotype calls, genotype likelihoods were calculated rather than deterministic calls.

Long Range Phasing.

Long ranged phasing of all chip genotyped individuals was performed with methods described previously (Kong, A. et al. Nat Genet. 40, 1068-75 (2008); Kong, A. et al. Nature 467, 1099-103 (2010)). In brief, phasing is achieved using an iterative algorithm which phases a single proband at a time, given the available phasing information about everyone else that shares a long haplotype identically by state with the proband. Given the large fraction of the Icelandic population that has been chip typed accurate long range phasing is available genome-wide for all chip typed Icelanders.

Genotype Imputation.

We impute the SNPs identified and genotyped through sequencing into all Icelanders that have been phased using long range phasing using the model used by IMPUTE (Marchini, J. et al. Nat Genet. 39, 906-13 (2007)). The genotype data from sequencing can be ambiguous due to low sequencing coverage and is not phased. In order phase the sequencing genotypes an iterative algorithm was applied for each SNP with alleles 0 and 1. Let H be the long range phased haplotypes of the sequenced individuals and follow:
1. For each haplotype h in H, use the hidden Markov model of IMPUTE to calculate γ_{h,k} for every other k in H, a measure of how likely h is to have the same ancestral source as k.
2. For every h in H initialize the parameter θₕ which specifies how likely the 1 allele of the SNP is to occur on the background of h from the genotype likelihoods obtained from sequencing. If L₀, L₁ and L₂ are the likelihoods of the genotypes 0, 1 and 2 in the individual that carries h, then set

$${θ}_{h} = \frac{{L}_{2} + \frac{1}{2} {L}_{1}}{{L}_{2} + {L}_{1} + {L}_{0}} .$$

3. For every pair of haplotypes h and k in H that are carried by the same individual use the other haplotypes in H to predict the genotype of the SNP on the backgrounds of h and k:

$${τ}_{h} = {\sum }_{l ∈ H \backslash \{ h \}} {γ}_{h , l} {θ}_{l} and {τ}_{k} = {\sum }_{l ∈ H \backslash \{ k \}} {γ}_{k , l} {θ}_{l} .$$

Combining these predictions with the genotype likelihoods from sequencing gives un-normalized updated phased genotype probabilities: P₀₀=(1−τₕ)(1−τₖ)L₀, P₀₁=(1−τₕ)τₖ½L₁, P₁₀=τₕ(1−τₖ)½L₁ and P₁₁=τₕτₖL₂. Now use these values to update θₕ and θₖ to

$$\frac{{P}_{10} + {P}_{11}}{{P}_{00} + {P}_{01} + {P}_{10} + {P}_{11}} and \frac{{P}_{01} + {P}_{11}}{{P}_{00} + {P}_{01} + {P}_{10} + {P}_{11}} ,$$

respectively.
4. Repeat step 3 while the maximum difference between iterations is greater than ε. We used ε=10⁻⁷.

Given the long range phased haplotypes and θ the allele of the SNP on a new haplotype h, not in H, is imputed as

$${\sum }_{l ∈ H} {γ}_{h , l} {θ}_{l} .$$

The above algorithm can easily be extended to handle simple family structures such as parent offspring pairs and triads by letting the P distribution run over all founder haplotypes in the family structure. The algorithm also extends trivially to the X-chromosome. If source genotype data is only ambiguous in phase, such as chip genotype data, then the algorithm is still applied but all but one of the Ls will be 0.

Association Testing.

Logistic regression was used to test for association between SNPs and disease, treating disease status as the response and expected genotype counts from imputation or allele counts from direct genotyping as covariates. Testing was performed using the likelihood ratio statistic.

Association Testing of Uric Acid Levels.

For each SNP a classical linear regression using the genotype as an additive covariate and Uric Acid as a response, was fitted to test for association. In addition to testing the standardized value, we also performed an analysis using log-transformed values which we then back-transformed to report the effect under a multiplicative model. We report significance levels based on the standardized values and the association effect based on both the standardized value and under the multiplicative model.

TABLE 1
Association results for markers on chromosome 19q13 with Gout.

| | | | | | | | | Seq |
|---|---|---|---|---|---|---|---|---|
| | | | | | | Effect | Other | ID |
| Marker | Position | P-corr | OR | Nimptd | Freq | Allele | Allele | NO: |
| chr19:54660818 | 54,660,818 | 2.70E−14 | 4.26 | 37438 | 0.018971 | G | C | 1 |
| chr19:54676721 | 54,676,721 | 2.23E−11 | 7.10 | 37440 | 0.008044 | C | T | 5 |
| chr19:54812310 | 54,812,310 | 4.57E−11 | 4.64 | 37431 | 0.012041 | G | A | 6 |
| chr19:54788061 | 54,788,061 | 4.60E−11 | 4.81 | 37432 | 0.010742 | C | G | 7 |
| chr19:54818578 | 54,818,578 | 5.25E−11 | 5.88 | 37428 | 0.008724 | A | G | 8 |
| chr19:54628061 | 54,628,061 | 4.83E−10 | 4.71 | 37444 | 0.014375 | C | G | 9 |
| chr19:54505919 | 54,505,919 | 1.15E−09 | 4.34 | 37452 | 0.012604 | C | A | 10 |
| chr19:55483086 | 55,483,086 | 5.89E−09 | 4.46 | 37424 | 0.008649 | C | T | 11 |
| chr19:55268031 | 55,268,031 | 1.04E−08 | 4.55 | 37419 | 0.00822 | C | T | 12 |
| chr19:55576372 | 55,576,372 | 1.52E−08 | 4.33 | 37417 | 0.008449 | T | C | 13 |
| chr19:55456500 | 55,456,500 | 1.64E−08 | 3.10 | 37418 | 0.023743 | T | C | 14 |
| chr19:54991872 | 54,991,872 | 2.05E−08 | 4.06 | 37421 | 0.011225 | G | A | 15 |
| chr19:55071043 | 55,071,043 | 2.09E−08 | 4.06 | 37426 | 0.011111 | G | T | 16 |
| chr19:55071103 | 55,071,103 | 2.09E−08 | 4.06 | 37426 | 0.011111 | C | G | 17 |
| chr19:55068782 | 55,068,782 | 2.17E−08 | 4.05 | 37428 | 0.011113 | T | C | 18 |
| chr19:55018776 | 55,018,776 | 2.20E−08 | 4.06 | 37431 | 0.011222 | A | G | 19 |
| chr19:55471711 | 55,471,711 | 2.80E−08 | 3.77 | 37428 | 0.013484 | C | A | 20 |

Shown are marker ID's (chromosome followed by location in NCBI Build 36), position in NCBI Build 36, P-value of association to Gout, OR, number of imputed individuals (cases and controls), frequency of risk allele in population, identity of risk allele, identity of alternate allele, SEQ ID for flanking sequence of the marker.

TABLE 2
Association results for markers on chromosome 19q13 with Uric Acid levels.

| | | | | | | | | | Seq |
|---|---|---|---|---|---|---|---|---|---|
| | | | | | | | Effect | Other | ID |
| Marker | Position | P-corr | Effect | Nimptd | Freq | Info | Allele | Allele | NO: |
| chr19:54660818 | 54,660,818 | 1.02E−13 | 0.339235 | 37438 | 0.018971 | 0.875 | G | C | 1 |
| chr19:54818578 | 54,818,578 | 1.01E−12 | 0.492249 | 37428 | 0.008724 | 0.808016 | A | G | 8 |
| chr19:54676721 | 54,676,721 | 2.64E−12 | 0.529883 | 37440 | 0.008044 | 0.73247 | C | T | 5 |
| chr19:54788061 | 54,788,061 | 1.11E−11 | 0.410939 | 37432 | 0.010742 | 0.877051 | C | G | 7 |
| chr19:55268031 | 55,268,031 | 2.59E−11 | 0.44035 | 37419 | 0.00822 | 0.958486 | C | T | 12 |
| chr19:55576372 | 55,576,372 | 3.30E−11 | 0.428137 | 37417 | 0.008449 | 0.982579 | T | C | 13 |
| chr19:55483086 | 55,483,086 | 4.04E−11 | 0.425225 | 37424 | 0.008649 | 0.965806 | C | T | 11 |
| chr19:54812310 | 54,812,310 | 8.53E−11 | 0.379461 | 37431 | 0.012041 | 0.852417 | G | A | 6 |
| chr19:54505919 | 54,505,919 | 9.35E−11 | 0.381575 | 37452 | 0.012604 | 0.817579 | C | A | 10 |
| chr19:54628061 | 54,628,061 | 1.67E−10 | 0.372086 | 37444 | 0.014375 | 0.710314 | C | G | 9 |
| chr19:55071103 | 55,071,103 | 8.77E−10 | 0.373215 | 37426 | 0.011111 | 0.853074 | C | G | 17 |
| chr19:55071043 | 55,071,043 | 8.77E−10 | 0.37211 | 37426 | 0.011111 | 0.85307 | G | T | 16 |
| chr19:55068782 | 55,068,782 | 8.90E−10 | 0.373083 | 37428 | 0.011113 | 0.852798 | T | C | 18 |
| chr19:54991872 | 54,991,872 | 9.37E−10 | 0.372572 | 37421 | 0.011225 | 0.843771 | G | A | 15 |
| chr19:55018776 | 55,018,776 | 1.02E−09 | 0.3714 | 37431 | 0.011222 | 0.844069 | A | G | 19 |
| chr19:54400812 | 54,400,812 | 1.68E−09 | 0.326239 | 37451 | 0.015703 | 0.763755 | T | C | 21 |
| rs73051934 | 53,274,728 | 1.57E−08 | 0.278195 | 37550 | 0.020134 | 0.777004 | G | A | 22 |
| chr19:55602702 | 55,602,702 | 4.61E−08 | 0.321619 | 37413 | 0.011001 | 0.945915 | G | A | 23 |

Shown are marker ID's (chromosome followed by location in NCBI Build 36), position in NCBI Build 36, P-value of association to uric acid levels, effect in fraction of standard deviations, number of imputed individuals (cases and controls), frequency of effect allele in population, information content of the imputation, identity of effect allele, identity of alternate allele, SEQ ID for flanking sequence of the marker.

### Example 2

Direct Sanger sequencing was performed on 724 individuals with Gout and 960 controls over the SG19S921 missense mutation in ALDH16A1. Of the 724 cases, about ⅔ (453) had also been chip-typed.

Results of association analysis of SG19S921 based on this data is shown in Table 3.

TABLE 3
Results of Sanger sequencing of the SG19S921 polymorphism in Gout
cases and controls. Shown are p-value of association, odds ratio (OR),
number of affected individuals sequenced, frequency of allele in
affecteds, number of controls sequenced, frequency of allele in
controls, identity of allele, and marker name.

| | | | | | Con | | |
|---|---|---|---|---|---|---|---|
| p-value | OR | # Aff | Aff freq | # Con | freq | Allele | Marker |
| 3.58 × | 0.203 | 724 | 0.941 | 960 | 0.988 | 2 | SG19S921 |
| 10−14 | | | | | | | |
| 5.91 × | 5.499 | 724 | 0.057 | 960 | 0.011 | 3 | SG19S921 |
| 10−15 | | | | | | | |
| 0.89 | 0.88 | 724 | 0.0014 | 960 | 0.0016 | 1 | SG19S921 |

These results confirm the finding that the G allele of SG19S921, encoding the P527R variant in ALDH16A1 confers significant increased risk of Gout.

These results also suggest that a third allele (A) may occur at this nucleotide position. The allele is however very rare and does not appear to correlate with risk of Gout.

1. A method of determining a susceptibility to Gout, the method comprising:
analyzing nucleic acid from a biological sample from a human individual to obtain nucleic acid sequence data for at least one at-risk allele of at least one polymorphic marker in the human ALDH16A1 gene wherein different alleles of the at least one polymorphic marker are associated with different susceptibilities to Gout in humans, and
determining a susceptibility to Gout for the human individual from the nucleic acid sequence data.

2-3. (canceled)

4. The method of claim 1, wherein the nucleic acid sequence data is obtained using a method that comprises at least one procedure selected from:
(i) amplification of nucleic acid from the biological sample;
(ii) hybridization assay using a nucleic acid probe and nucleic acid from the biological sample;
(iii) hybridization assay using a nucleic acid probe and nucleic acid obtained by amplification of the biological sample, and
(iv) high-throughput sequencing.

5-8. (canceled)

9. The method of claim 1, wherein the at least one polymorphic marker is a marker that encodes a defective ALDH16A1 protein.

10. The method of claim 9, wherein the at least one polymorphic marker encodes a missense variation, a nonsense variation, or a truncation in an ALDH16A1 protein with sequence as set forth in SEQ ID NO: 3.

11. The method of claim 1, wherein the at least one polymorphic marker is a C/G single nucleotide polymorphism at position 54,660,818 in NCBI Build 36 (position 201 in SEQ ID NO:1).

12. The method of claim 11, wherein determination of the presence of a G allele in the single nucleotide polymorphism at position 54,660,818 in NCBI Build 36 (position 201 in SEQ ID NO:1) is indicative of an increased susceptibility of Gout for the human individual.

13. The method of claim 1, wherein the at least one polymorphic marker is rs150414818, and wherein a determination of the presence of allele G of rs150414818 in the human individual is indicative of increased susceptibility of gout for the individual.

14. A method of determining a susceptibility to Gout, the method comprising: analyzing amino acid from a biological sample from a human individual to obtain amino acid sequence data, wherein the amino acid sequence data comprises data about at least one missense variation, at least one nonsense variation, or at least one truncation in an ALDH16A1 protein, and
determining a susceptibility to Gout for the human individual from the amino acid sequence data.

15-21. (canceled)

22. A method of determining a susceptibility to Gout, the method comprising:
analyzing nucleic acid sequence data from a human individual for at least one polymorphic marker selected from the group consisting of:
C/G polymorphism at position 54,660,818 in NCBI Build 36 (SEQ ID NO:1);
C/T polymorphism at position 54,676,721 in NCBI Build 36 (SEQ ID NO:5)
A/G polymorphism at position 54,812,310 in NCBI Build 36 (SEQ ID NO:6)
C/G polymorphism at position 54,788,061 in NCBI Build 36 (SEQ ID NO:7)
A/G polymorphism at position 54,818,578 in NCBI Build 36 (SEQ ID NO:8)
C/G polymorphism at position 54,628,061 in NCBI Build 36 (SEQ ID NO:9)
A/C polymorphism at position 54,505,919 in NCBI Build 36 (SEQ ID NO:10)
C/T polymorphism at position 55,483,086 in NCBI Build 36 (SEQ ID NO:11)
C/T polymorphism at position 55,268,031 in NCBI Build 36 (SEQ ID NO:12)
C/T polymorphism at position 55,576,372 in NCBI Build 36 (SEQ ID NO:13)
C/T polymorphism at position 55,456,500 in NCBI Build 36 (SEQ ID NO:14)
A/G polymorphism at position 54,991,872 in NCBI Build 36 (SEQ ID NO:15)
G/T polymorphism at position 55,071,043 in NCBI Build 36 (SEQ ID NO:16)
C/G polymorphism at position 55,071,103 in NCBI Build 36 (SEQ ID NO:17)
C/T polymorphism at position 55,068,782 in NCBI Build 36 (SEQ ID NO:18)
A/G polymorphism at position 55,018,776 in NCBI Build 36 (SEQ ID NO:19)
A/C polymorphism at position 55,471,711 in NCBI Build 36 (SEQ ID NO:20),
wherein different alleles of the at least one polymorphic marker are associated with different susceptibilities to Gout in humans, and
determining a susceptibility to Gout from the nucleic acid sequence data.

23-30. (canceled)

31. A method of predicting prognosis of an individual diagnosed with Gout, the method comprising
obtaining sequence data about a human individual about at least one polymorphic marker in the human ALDH16A1 gene, wherein different alleles of the at least one polymorphic marker are associated with different susceptibilities to Gout in humans, and
predicting prognosis of Gout from the sequence data.

32-33. (canceled)

34. A method of assessing probability of response of a human individual to a therapeutic agent for preventing, treating and/or ameliorating symptoms associated with Gout, comprising:
obtaining nucleic acid sequence data about a human individual identifying at least one allele of at least one polymorphic marker in the human ALDH16A1 gene, wherein different alleles of the at least one polymorphic marker are associated with different probabilities of response to the therapeutic agent in humans, and
determining the probability of a positive response to the therapeutic agent from the sequence data.

35. The method of claim 34, wherein the at least one therapeutic agent is selected from the group consisting of a non-steriod anti-inflammatory drug, a glucocorticoid, colchicine (N-[(R7S)-1,2,3,10-tetramethoxy-9-oxo-5,6,7,9-tetrahydrobenzo[a]heptalen-7-yl]acetamide), pegloticase, allopurinol (3,5,7,8-tetrazabicyclo[4.3.0]nona-3,5,9-trien-2-one), probenecid (4-(dipropylsulfamoyl)benzoic acid) and febuxostat (2-(3-cyano-4-isobutoxyphenyl)-4-methyl-1,3-thiazole-5-carboxylic acid).

36. The method of claim 34, wherein the at least one polymorphic marker encodes a missense variation, a nonsense variation, or a truncation in an ALDH16A1 protein with sequence as set forth in SEQ ID NO: 3.

37. The method of claim 34, wherein the at least one polymorphic marker is a C/G single nucleotide polymorphism at position 54,660,818 in NCBI Build 36 (SEQ ID NO:1), and wherein a determination of the presence of allele G in the polymorphic marker is indicative of a positive response to the therapeutic agent for the individual.

38. A kit for assessing susceptibility to Gout in human individuals, the kit comprising:
reagents for selectively detecting at least one at-risk variant for Gout in the individual, wherein the at least one at-risk variant is a variant in the human ALDH16A1 gene or an encoded ALDH16A1 protein that is associated with risk of Gout in humans, and
a collection of correlation data between the at least one at-risk variant and susceptibility to Gout.

39-56. (canceled)

57. An apparatus for determining a susceptibility to Gout in a human individual, comprising:
a processor;
a computer readable memory having computer executable instructions adapted to be executed on the processor to analyze sequence information about at least one human individual with respect to at least one marker in the human ALDH16A1 gene or an encoded human ALDH16A1 protein that is associated with susceptibility of Gout in humans, and generate an output based on the marker sequence information, wherein the output comprises at least one measure of susceptibility to Gout for the human individual.

58-62. (canceled)

63. A system for identifying susceptibility to gout in a human subject, the system comprising:
at least one processor;
at least one computer-readable medium;
a susceptibility database operatively coupled to a computer-readable medium of the system and containing population information correlating the presence or absence of one or more alleles of the human ALDH16A1 gene and susceptibility to gout in a population of humans;
a measurement tool that receives an input about the human subject and generates information from the input about the presence or absence of at least one mutant ALDH16A1 allele indicative of a ALDH16A1 defect in the human subject; and
an analysis tool that:
is operatively coupled to the susceptibility database and the measurement tool,
is stored on a computer-readable medium of the system,
is adapted to be executed on a processor of the system, to compare the information about the human subject with the population information in the susceptibility database and generate a conclusion with respect to susceptibility to gout for the human subject.

64. The system according to claim 63, further including:
a communication tool operatively coupled to the analysis tool, stored on a computer-readable medium of the system and adapted to be executed on a processor of the system to communicate to the subject, or to a medical practitioner for the subject, the conclusion with respect to susceptibility to gout for the subject.

65. The system according to claim 63, wherein the at least one mutant ALDH16A1 allele is indicative of a defect in an encoded ALDH16A1 protein selected from the group consisting of: a missense substitution in ALDH16A1, a nonsense substitution in ALDH16A1 and a truncation in ALDH16A1.
wherein mutant alleles indicative of the defect are associated with increased susceptibility to gout.

66. The system according to claim 65, wherein the at least one mutant allele is the G allele of a C/G single nucleotide polymorphism at position 54,660,818 in NCBI Build 36 (position 201 in SEQ ID NO:1).

67. The system according to claim 63, wherein the measurement tool comprises a tool stored on a computer-readable medium of the system and adapted to be executed by a processor of the system to receive a data input about a subject and determine information about the presence or absence of the at least one mutant ALDH16A1 allele in a human subject from the data.

68. The system according to claim 67, wherein the data is genomic sequence information, and the measurement tool comprises a sequence analysis tool stored on a computer readable medium of the system and adapted to be executed by a processor of the system to determine the presence or absence of the at least one mutant ALDH16A1 allele from the genomic sequence information.

69. The system according to claim 63, wherein the input about the human subject is a biological sample from the human subject, and wherein the measurement tool comprises a tool to identify the presence or absence of the at least one mutant ALDH16A1 allele in the biological sample, thereby generating information about the presence or absence of the at least one mutant ALDH16A1 allele in a human subject.

70. The system according to claim 69, wherein the measurement tool includes:
an oligonucleotide microarray containing a plurality of oligonucleotide probes attached to a solid support;
a detector for measuring interaction between nucleic acid obtained from or amplified from the biological sample and one or more oligonucleotides on the oligonucleotide microarray to generate detection data; and
an analysis tool stored on a computer-readable medium of the system and adapted to be executed on a processor of the system, to determine the presence or absence of the at least one mutant ALDH16A1 allele based on the detection data.

71. The system according to claim 69, wherein the measurement tool includes:
a nucleotide sequencer capable of determining nucleotide sequence information from nucleic acid obtained from or amplified from the biological sample; and
an analysis tool stored on a computer-readable medium of the system and adapted to be executed on a processor of the system, to determine the presence or absence of the at least one mutant ALDH16A1 allele based on the nucleotide sequence information.

72. The system according to claim 63, further comprising:
a medical protocol database operatively connected to a computer-readable medium of the system and containing information correlating the presence or absence of the at least one mutant ALDH16A1 allele and medical protocols for human subjects at risk for gout; and
a medical protocol routine, operatively connected to the medical protocol database and the analysis routine, stored on a computer-readable medium of the system, and adapted to be executed on a processor of the system, to compare the conclusion from the analysis routine with respect to susceptibility to gout for the subject and the medical protocol database, and generate a protocol report with respect to the probability that one or more medical protocols in the database will:
reduce susceptibility to gout; or
delay onset of gout; or
increase the likelihood of detecting gout at an early stage to facilitate early treatment.

73. The system according to claim 64, wherein the communication tool is operatively connected to the analysis routine and comprises a routine stored on a computer-readable medium of the system and adapted to be executed on a processor of the system, to:
generate a communication containing the conclusion; and
transmit the communication to the subject or the medical practitioner, or enable the subject or medical practitioner to access the communication.

74. The system according to claim 73, wherein the communication expresses the susceptibility to gout in terms of odds ratio or relative risk or lifetime risk.

75. The system according to claim 63, further comprising:
a medical protocol database operatively connected to a computer-readable medium of the system and containing information correlating the presence or absence of the at least one mutant ALDH16A1 allele and medical protocols for human subjects at risk for gout; and
a medical protocol routine, operatively connected to the medical protocol database and the analysis routine, stored on a computer-readable medium of the system, and adapted to be executed on a processor of the system, to compare the conclusion from the analysis routine with respect to susceptibility to gout for the subject and the medical protocol database, and generate a protocol report with respect to the probability that one or more medical protocols in the database will:
reduce susceptibility to gout; or
delay onset of gout; or
increase the likelihood of detecting gout at an early stage to facilitate early treatment, and
wherein the communication further includes the protocol report.

76. The system according to claim 63, wherein the susceptibility database further includes information about at least one parameter selected from the group consisting of age, sex, ethnicity, race, medical history, weight, blood pressure, family history of gout, and smoking history in humans and impact of the at least one parameter on susceptibility to gout.

77. A system for assessing or selecting a treatment protocol for a subject diagnosed with gout, comprising:
at least one processor;
at least one computer-readable medium;
a medical treatment database operatively connected to a computer-readable medium of the system and containing information correlating the presence or absence of at least one mutant ALDH16A1 allele and efficacy of treatment regimens for gout;
a measurement tool to receive an input about the human subject and generate information from the input about the presence or absence of the at least one mutant ALDH16A1 allele indicative of a ALDH16A1 defect in a human subject diagnosed with gout; and
a medical protocol tool operatively coupled to the medical treatment database and the measurement tool, stored on a computer-readable medium of the system, and adapted to be executed on a processor of the system, to compare the information with respect to presence or absence of the at least one mutant ALDH16A1 allele for the subject and the medical treatment database, and generate a conclusion with respect to at least one of:
the probability that one or more medical treatments will be efficacious for treatment of gout for the patient; and
which of two or more medical treatments for gout will be more efficacious for the patient.

78. The system according to claim 77, wherein the measurement tool comprises a tool stored on a computer-readable medium of the system and adapted to be executed by a processor of the system to receive a data input about a subject and determine information about the presence or absence of the at least one mutant ALDH16A1 allele in a human subject from the data.

79. The system according to claim 77, wherein the data is genomic sequence information, and the measurement tool comprises a sequence analysis tool stored on a computer readable medium of the system and adapted to be executed by a processor of the system to determine the presence or absence of the at least one mutant ALDH16A1 allele from the genomic sequence information.

80. The system according to claim 77, wherein the input about the human subject is a biological sample from the human subject, and wherein the measurement tool comprises a tool to identify the presence or absence of the at least one mutant ALDH16A1 allele in the biological sample, thereby generating information about the presence or absence of the at least one mutant ALDH16A1 allele in a human subject.

81. The system according to claim 77, further comprising a communication tool operatively connected to the medical protocol routine for communicating the conclusion to the subject, or to a medical practitioner for the subject.

82. The system according to claim 81, wherein the communication tool comprises a routine stored on a computer-readable medium of the system and adapted to be executed on a processor of the system, to:
generate a communication containing the conclusion; and
transmit the communication to the subject or the medical practitioner, or enable the subject or medical practitioner to access the communication.

83. The system according to claim 77, wherein the at least one mutant ALDH16A1 allele is indicative of a defect in an encoded ALDH16A1 protein selected from the group consisting of: a missense substitution in ALDH16A1, a nonsense substitution in ALDH16A1 and a truncation in ALDH16A1.
wherein mutant alleles indicative of the defect are associated with increased susceptibility to gout.

84. The system according to claim 83, wherein the at least one mutant allele is the G allele of a C/G single nucleotide polymorphism at position 54,660,818 in NCBI Build 36 (position 201 in SEQ ID NO:1).


SEQUENCE LISTING (23 sequences)

SEQ ID NO: 1 - DNA, 401 bp, Homo sapiens
tggagtttagggtcccccagtgaatcatgggagcaggagttttagggtcccccagaggctcatgggaacaggggtttggggtgccccagaggctcacgggagccaaggcagtcttcccagtgcggtagagcatagggtctgagcagattgaggccccaggactcctcctcatgtcccaccctactccagcccagcaccccsctatgggctcttcgttgggggccgtttccaggctcctggggcccgaagctccaggcccatccgggattcgtctggcaacctccatggctacgtggctgagggtggagccaaggacatccgaggtgctgtggaggccgctcaccaggctttccctgggtaaggggtcacacgggaaagcccaagggtcatggtgtggcaga

SEQ ID NO: 2 - protein, 751 aa, Homo sapiens
MAATRAGPRAREIFTSLEYGPVPESHACALAWLDTQDRCLGHYVNGKWLKPEHRNSVPCQDPITGENLASCLQAQAEDVAAAVEAARMAFKGWSAHPGVVRAQHLTRLAEVIQKHQRLLWTLESLVTGRAVREVRDGDVQLAQQLLHYHAIQASTQEEALAGWEPMGVIGLILPPTFSFLEMMWRICPALAVGCTVVALVPPASPAPLLLAQLAGELGPFPGILNVLSGPASLVPILASQPGIRKVAFCGAPEGGLRLLIQESVWDEAMRRLQERMGRLRSGRGLDGAVDMGARGAAACDLVQRFVREAQSQGAQVFQAGDVPSERPFYPPTLVSNLPPASPCAQVEVPWPVVVASPFRTAKEALLVANGTPRGGSASVWSERLGQALELGYGLQVGTVWINAHGLRDPSVPTGGCKESGCSWHGGPDGLYEYLRPSGTPARLSCLSKNLNYDTFGLAVPSTLPAGPEIGPSPAPPYGLFVGGRFQAPGARSSRPIRDSSGNLHGYVAEGGAKDIRGAVEAAHQAFPGWAGQSPGARAALLWALAAALERRKSTLASRLERQGAELKAAEAEVELSARRLRAWGARVQAQGHTLQVAGLRGPVLRLREPLGVLAVVCPDEWPLLAFVSLLAPALAYGNTVVMVPSAACPLLALEVCQDMATVFPAGLANVVTGDRDHLTRCLALHQDVQAMWYFGSAQGSQFVEWASAGNLKPVWASRGCPRAWDQEAEGAGPELGLRVARTKALWLPMGD

SEQ ID NO: 3 - protein, 802 aa, Homo sapiens
MAATRAGPRAREIFTSLEYGPVPESHACALAWLDTQDRCLGHYVNGKWLKPEHRNSVPCQDPITGENLASCLQAQAEDVAAAVEAARMAFKGWSAHPGVVRAQHLTRLAEVIQKHQRLLWTLESLVTGRAVREVRDGDVQLAQQLLHYHAIQASTQEEALAGWEPMGVIGLILPPTFSFLEMMWRICPALAVGCTVVALVPPASPAPLLLAQLAGELGPFPGILNVLSGPASLVPILASQPGIRKVAFCGAPEEGRALRRSLAGECAELGLALGTESLLLLTDTADVDSAVEGVVDAAWSDRGPGGLRLLIQESVWDEAMRRLQERMGRLRSGRGLDGAVDMGARGAAACDLVQRFVREAQSQGAQVFQAGDVPSERPFYPPTLVSNLPPASPCAQVEVPWPVVVASPFRTAKEALLVANGTPRGGSASVWSERLGQALELGYGLQVGTVWINAHGLRDPSVPTGGCKESGCSWHGGPDGLYEYLRPSGTPARLSCLSKNLNYDTFGLAVPSTLPAGPEIGPSPAPPYGLFVGGRFQAPGARSSRPIRDSSGNLHGYVAEGGAKDIRGAVEAAHQAFPGWAGQSPGARAALLWALAAALERRKSTLASRLERQGAELKAAEAEVELSARRLRAWGARVQAQGHTLQVAGLRGPVLRLREPLGVLAVVCPDEWPLLAFVSLLAPALAYGNTVVMVPSAACPLLALEVCQDMATVFPAGLANVVTGDRDHLTRCLALHQDVQAMWYFGSAQGSQFVEWASAGNLKPVWASRGCPRAWDQEAEGAGPELGLRVARTKALWLPMGD

SEQ ID NO: 4 - DNA, 17833 bp, Homo sapiens
attcccattagccccgcccctttgggctggaaccggaggtgtcgctcttcggacctcaaggttccccttaacacagagcgccccgcagtcttcgcggaaagcgttcggggtaggcgatggctgcgacgcgtgcagggccccgcgcccgcgagatcttcacctcgctggagtacggaccggtgccggagagccacgcatgcgcactggtgagagtctgcccggccggcgctgctcgctgcgttccccaggcctgggcgcccggtttttcgcggggagtcccgtcatccactgcggtagctcagccgctccgcctctcttagtccccgtgattcccgccgcccaataggatcgcgccctgtaggacgctcccttgagccttggcggtggcagccttttagtctgttccggtcttccccactggttctcttgccccttgatcctgaactcctcgtagtgtttccgcggttttcctgaactcctctagacgctctcgtgattcctcagggtcccctcagaacctgacgccacccaccagaccccttaggattcctgtgaggctccagtgacccacaacccccattgtctttccacgactcttcaaaacatcctatggcttccatgggcctccagagcaccccagacctcctgagggactcccttcatccctttgattacccagagaccttcagaacttccatggagcccccgtgatcccataggacccctcaatactcgtggggttcatgtgacctcatcggaactctcaggagcctagaatacatcaggacttccggtttgggttgggttttttttttgtttttttttttgagccagggtctcactctgtcgcccaaactgaagtgcactggcgctatcacaccactgcagcctcaacctcctgggctcaagccatcctctcacctcagccccccaagtaactgggaccagagacgcgcgccaccactctcagctaatatttgtactttttctagagacaaggtttcagcatgttggccaggctggtctcgaactcctggcctcaagtgatccacccacctcgacttcccaaagtgctgggattacaggcgtgagctactgcacccagccacatcaggacttcctgtaattacttatggcccctcacaataccctcacggggctcctatgatctctctagctctcagaatgtcccatagggttcccatgattgttcaggaacccctgaagtcctcgtgagggtcccatgatcccctgggggtcctcagaacctccctaggactcccatgacccctcagaggcccccagaaccctcgtggggttcctgcaaatgagtagggcacctcaaactttctgtcgtttccccaggcactctcagaattccttcacggggctctcatgatcccccagggtcccccagcacccaggcgaaaggagcttaggcttctttgagggggagggcaggaggccagagcaatgatccaagtggaaggaactggatcagaaccctggcacagcctagaacttggggaggccaagacgggcagatcgcctgagctcaggaattcgagaccagcctgggcaacatagtgaaacctcgtctctactaaaatacaaaacattgggccaggcgtggtggctcagacttgtaatcccagcactttgggagtcctaggtgggcggatcacctgaggtcaggagttcgagaccagcctggccaacatggtgaaaccccatctctactaaaatacaaaaattacctgagcgtggtggtgtttgcctgtaatcctagctacttgggaggctgaggtgggagaattgcttgaatccaggaggcagaggttgcagtaagctgagattgcaccactgcactccagcctgggcaacggagcaagacgctgtctccaaaaaaaatacaaaaatggctgggcgcagtggttcacacctgtaatcccagcgctttgggaggccgaggcgggcggatcatgaggtcaggagatcgagaccagcctggccaagatggtgaaacccatctctactaaaaatacaaaaaattagctgggcgtggtggcaggcacctgtaatcccagctacttgggaggctggggcaggagaatcgcttgaacccaggaggtggagattgcagtgagccgagaccacgccactgcactccagcctgggcaacaagagcaaaactccatctcaaaaaaaaaaaaaaaaaaaaaaggaaacagaaaacaaaagattagccgaggatggcagcacacacctgtagtcccagctacccgggaggctgaggtgggaggatcacctgagcctgggaggtgcagtgatcataccactgaactccagcctgggtgacagaataagaccctgtcccccctgaaaaaaaaagaagttgaggccaggcaagtggctcacacctgtaatcccagcagtttgggagaccgaggcaggaggatcactcgcacctaggagtttgagaccagcctgggcaacatggtgaggccctatctctattaatataaaaataaaacataaaaagtgaagttgagacttggacttgagtccttgcatttcagacccgtggtcttgcctacaccagcaaagttctcaaccgtacagtctctgggccagactcaaccaacaaacatgttttctttggcgtgaactttattttttttttatgtgaaatgctggacaattgattccatgtaaacatccgggaggtcctcttgaaaataaaattccaaaatgtgtcaacatttggctcaacttccctgacaaaagtcatctgggaccaatagctcctgtcttttccatgaatcgttaagctcctcttctcatccaggccctcttctcaggcctcctggaaccctctggacattcttttggaattttgtttttcttttggggggaagctttccaacttgcacagaataattagcatttattttgaactcctgtttacccatcactgtgcctcaaccatcatcaacatttatcccagcgctgtttccttctgtattttctttggagtaatttaaacaaatcctggatgtcacagtattttcctataaatatttgaatcctttcttctaacagataagggctcattttgttacaaaactaccatggtctgttaccacacttaacattaagtcattctttttgttttttgtttgtttgtttgagacaggatcttactctgtcgcccaggctagcattcagtagcgttcatagctcactgcagcctgaaactcctggacccaagcaatcctcccaccccagcctcctgactagctgagactacaagtgtgcatcatcatgactggcaaattttttacttttatttttgtagagatggggtcttgctgtgtaggtccagactagtctcaaactcctcagctcaagcaatcctcccgtctcagcgtgggaaagtgcagggattacagatgtgagctaccatgcccggccaacaatcattctttaatgtcatctgatgcccagttcatgttcaaatatccctgactttctcacagatgtctctctccctgtgggcgtttgagtttggaacttctaagtaacttttttgctttaaagagaaaggagaagccaggcacagtggctcatgcttgtaatcccagcactttgggaggccgaggcgagtggatcacctgaggccaggagttcgagaccagcctggccaacatggtaaaaacccatctctactagaaatatataaattagctgggcatggtggcggccgcctgtaatcccagctacttgggaggctgaggtaggagaatcacgtgaacccaggaggcggaggttgcagtgagctgagatcgcaccattgcactccagcctgggagacaagagcaagactccatctcaaaaaaaaaaaagaaagaagggccagtaagagaaaaatcaggaatatgtgacatcagagatgctaggggagtctgtagaaggagcaagtgagcagtgtgctttggatttagcatcagggaagacgtgaccaggatgagaaagaaatatggaggagaagtccgggcgcagtggctcacgcctgtaatctcagcactttgggaggccgaggcgggcggatcacgaggtcaggagatcaagaccatcctggctaacacagtgaaaccccatctctactaaaaatacaaaaaaattagccaggcgtggtggcaggcgcatgtagtcccagctactcgggagcctgaggcaggagaatggcgtgaatccaggaggcggaccttgcagtgagcagagatcgcgccactgcactccagcctgggtgacagagcaagactctgtctcaaacaaaaaaaaaaaaaagaaagaaagaaatatggaggagatgtggtgagtgagatgtggtaaaaaaattttttttctttgagacagagccttgctctgttactcaggctggagtgcagtggcgtgatcttggcttactacagcctctacctcctgggctcaagtgatcctcccacctcagactcccaggtagctgggactacaggcactcaccaccacacccggctaactttttgtattttttgtagagatgagctttcaccatgttgcccaggctggtcttgaatgcctaggctcagaccacctcggcctcccaaagtggactccaaataggtgccactgttgtggcaccagcccaaggactcagttgagaccctaattgaatgaattaaagatgttgacacatccataatcccagcactttgggaggccaaggcgtgcagatcacttgaggtcaggagttcaagaccagcctgaccaacatggtgaaaccccgtctctactaaaaatacaaaaacaaaactagccgcatgtggtggcgggcgcctgtagtcccagctactcaggagattgaggcgggagaatagcgtgaacccaggaggcggagcttgcagtgagctgagatcatgccactgcactccagcctgggtgacagagcaagactcagtctcaaaaaaaaaaaatgaaaaaatgttctagaactagagtggtgatggttacacaacactgtaaatgcacagaatgccactaaactgctgaactatacactttgaaatgctctattttaccacaaactttttttttaaagcaataggaggaaaagaattagctgatggggcagagggagacagacgtcccctcctagaggattctgggtaggggttgaactgccccagctcctcccgaacccctttccaaactgtctctacctcccccaggcctggctggacacccaggaccggtgcttgggccactatgtgaatgggaagtggttaaagcctgaacacagaaattcagtgccttgccaggatcccatcacaggtacgagatgtcccccagtcattagtggaagggaggtatctgtgccctctagccctacccaccaacacccattccccttgcctagggccctgagggcaggaaacagctggtggtgggagatgggctgcgatgtctggttccttctgaaaccagtcaccctaattatggttccgggtctctgtggaggctttgtgagactgctgtctgttaaaatagcttccttgccctggaggtgatcatagccgtgacccagcagagaatggggaccagctttacactcttgggctgatccaggctccgaaaatctcagccccctccagatatgacatgggctactcctcccatctgagtccccccacttttctccccaggagagaacttggccagttgcctgcaggcacaggccgaggatgtggctgcagccgtggaggcagccaggatggcatttaagggctggagtgcgcaccccggcgtcgtccgggcccagcacctgaccaggtgatgcagctgaggtgtggaccccgggaggcggggaaccccagcatccactcgagaccatgggaacaaaggctatttcccaagatcccactgaattaatttgcagctaaggctcagattcccagtatgtgctggaggcagtcgtggctgaaacatgcatccgttgtccactattcactgggactagaagccacaaagcctctatttcccaggacattcttggagtgcattggggctcagttcactggggctggtgaagcccctaaatccatttcctagccgcttgcggggaagctagagacaaaaattccatttcccaaaacgcaattgaggttagaagctaagactcaatttcccaaaagcttcagggatttccggcggccaagacctttacacagcatcctcagggtttgtgtttccatgactataattctcataaatcttcactgccctctgccccaggtaaatggtggggatgcctcaggggctcatggggattgtagtccaggtatataggagcagcccaggactctgcaaggctgagggccgttggaaaatgagcaccctcttgctttctcgacaggctggccgaggtgatccagaagcaccagcggctgctgtggaccctggaatccctggtgactgggcgggctgttcgagaggttcgagacggggacgtccagctggcccagcagctgctccactaccatgcaatccaggcatccacccaggaggaggcactggcaggctgggagcccatgggtgagaccctggagtccctagccctatcctcccacatgactcagcagtgctagctccagtcccctcattctttttcttttagacagagtttcgctcttgtcgcccaggccggagtgcagtggcgcaatcttggctcactataacctccgcctcccgggttcaagtgattctcctgcctcagcctcctgagtagctggaattacaggcacccaccaccacacctggctaattgttatatttttattagagaagaggggtctcatcatgttggtcaggctggtctcaaactcctaacctcagatgattcacctgccttggcatcccaaagtgctgggattacaggcatgagccaccgtgcccggtcccctcattctttttcttttgtttttgtttttgtttttttctttttcagacagggtctttctctgtcaccctggctggagtacagtggtgcaattatagctcactgatacctcgacctcctgggctcaaaatcctcccacctcagcctcccaagtaactgggactacgaccatgcaccaccacacctgactagtttttgtttttgttttttttttttttgagatggagtttcgctctttgttgcccaggctggagtgcagtggtgcgatctcggctcactggaacctccgcctcccgtgttcaagtgattctcctgcctcagcctcccgagtagctgggactacaggcatgcaccaccacgcctggctaattttgtgtttttagtggagatggggtttttccatgttggtcaggctggtctcgaacttcccacctcaggtgatccgcccacctcgacctcccaaagtgctgagattacaggcgtgagccaccacacccggccatttttttttttttttcctaatgtagccatggggtcttgccatgtggcccaggctggacttaaacacctagcctcaagggatcctcttgcctcatttttttcttgcaggagtaattggcctcatcctgccacccacattctccttccttgagatgatgtggaggatttgccctgccctggctgtgggtaaatgatggcctggggggtcctgactcttgggtctgagaaaggaggggatcgtggggcccaaactccagagtctgagagacaagggggctggaggcctggactctgtggaaggagtctgtgggaggaggggctgcgggtctgaactcctaggtctgagggaggaggagctggagtctggactcctgagtctgagggaggaggagctggagtctggactcctgagtctgagggaggaggggctggagtctggactcctgggtctgagggaggaggggctggggtctggattcctgggtctgagggaggaggggctggggtctggactcctgggtgtgagggaggaggggctggggtctggactcctgggtctgagggaggaggggctgggacctggactcctgggtctgagggaggaggggctgggacctggactcctgggtctgagggaggaggggctggagtctggactcctgggtctgagggaggaggggctggagtctggactcctgggtctgagggaggaggggctgggcctggactccggggtctgagggaggaggggctgggggcctggactcctgagtctgagggaggaggggctggagtctggactcctgggtctgagggaggaggggctgggggtccagattcctgggtctgagggaggaggggctgggcctggactcctgggcctttgagctgccccacttccccaggctgcaccgtggtggccctcgtgcccccggcctccccggcgcccctcctcctggcccagctggcgggggagctgggccccttcccgggaatcctgaatgtcctcagtggccctgcgtccctggtgcccatcctggcctcccagcctggaatccggaaggtggccttctgcggagccccggaggtaccttcgggacaggggtcgtggcggaacgcggctgggggccgcaaggctcctcctgcggctgaactggggggggtccctaggaagggcgtgcccttcgacggagcctggcgggagagtgtgcggagctgggcctggcgctggggacggagtcgctgctgctgctgacggacacggcggacgtagactcggccgtggagggtgtcgtggacgccgcctggtccgaccgcggcccggtgagacccgtgcgctcccgtctcctcataccctggaggccgttggtgtctgtctccagtcttcggggtcccgagtctctgttcattttattttattttattttatttattttgagacggagtttcactcttgtcgcccaggctggggtgcagcggcgcgattttggctcaccgcaacctctgcctcccaggttcagcctgggagtagctgcctcagcctcccgagtagctgggattacaggcgggcaccaccatgcctggctaatttatgtatttttagtagagacggggtttcaccatgttggccaggctggtctcgaactcctgacctcaggtgatccgcccacctcggcctcccaaagtgctgggactgcaggtgtgagccaccgcatccggcctctctgttctttaggactctgttgatttatctgagtgtccatgtccctagttttccagcctctgtcttcactcttcagatcaccaacttgctagagtgcaatggtgtgatctcctgatgccccttttcctcacagggtggcctcaggctcctcatccaggagtctgtgtgggatgaagccatgagacggctgcaggagcggatggggcggcttcggagtggccgagggctggatggggccgtggacatgggggcccggggggctgccgcatgtgacctggtccagcgctttgtgcgtgaggcccagagccagggtgcacaggtgaggcagggggtagagacttgagggtgtcaggggaggaggggcctggagacccagctcctgggtccaagggaggagggagctgggcgtggacaactgggtccgaggaaggaggggctgggagcctggactcctgggtctgaaggaggaggggctgggcctggactcctgggtctgagggaggaggtgctggggcctggattcctgggtctgagggaggaggggctggggcctggactcctgggtctgagggaggaggggctgggcctggactcctgggtctgagggaggaggtgctggggcccggattcctgggtctgagggaggaggggctggggcccggattcctgggtctgagggaggaggggctgggggtccagactcctgggtctgagggaggaggggctggcggcctggactcctgggtctgagggaggagaggctgggggtccagactcctgggtctgagggaggaggggctgggggcctggactcctgggtctgagggaggaggggctgggcctggactcctgggtctgagggaggaggtgctggggcctggattcctgggtctgagggaggaggggctggggcctggactcctgggtctgagggaggaggggctgggggtccagattcctgggtctgagggaggaggggctgggggcctggactcctgggtctgagggaggagaggctgggggtccagactcctgggtctgagggaggaggggctgggggcctggactcctgggtctgagggaggaggggctgggcctggactcctgggtctgagggaggaggtgctggggcctggattcctgggtctgagggaggaggggctggggcctggactcctgggtctgagggaggaggggctgggggtccagactcctgggtctgagggaggaggggctgggggcctggactcctgggtctgagggaggagaggctgggggtccagactcctgggtctgagggaggaggggctgggggcctggactcctgggtctgagggaggaggggctggggtcctgcagtcctcggtctcagcaggaagggaccagaagtcgacttctagatcatttctctccctaggtgttccaggctggtgatgtgccttcggaacgcccattctatcccccaaccttggtctccaacctgcccccagcctccccatgtgcccaggtggaggtgagacccttaaggctgcagagctcctacccaccgccagccaagggcagcagctctgtgcctggggaagcccttggggtaaccatgtgacctgggcgtgggggctgctgcctgctctaggctccttccctggtgggtgggccctggagggctgagcctcccggtcaccccttgcaggtgccgtggcctgtggtcgtggcctcccccttccgcacagccaaggaggcactgttggtggccaacgggacgccccgcgggggcagcgccagtgtgtggagcgagaggctggggcaggcgctggagctgggctatgggtcagtctgcgtggcccgggcgctcactcctctcctcattcttccatcttcatctcccttctccctgggtcgctccccgcgcctttaactcacccctctcccggcctgccaccgtctgttttcctctgttggacaccttcatcttccccacaggctccaggtgggcactgtctggatcaacgcccacggcctcagagacccttcggtgcccacaggcggctgcaaggagagtgggtgttcctggcacgggggcccagacgtgagtacatcccctccccgtcaccagcccccccgccgccccatgcccttgacactcgcatccggcctcgcgtgccccttgcatcctcttgacaccgtccctctcacaggggctgtatgagtatctgcggccctcagggacccctgcccggctgtcctgcctctccaagaacctgaactatgacacctttggcctcgctgttccctcaaccctgccggctgggcctgaaatagggcccaggtgagtcgttgggggccagtggtctgggagtgtgaacggggagaggggaattgatgcctgtttccccgtggactggagggctggcgcgaggtccatccaaaccatctcttagtcctgctgagtaccacgttgtggcctcacactttccctaccccaggcctgccgagactctcagctctgccccatgaatgtctgtactctcccagaaactcatgggtctaggagcttggggtcccccagaggttcactggagcagaagcttggggtccctcagaggctcatgggagcaggagcttggggtcccccagaggctcatgggagcagaaacttggggtcctccagaggctcatgggagcaggagtttgaggtccccccagagactcaagggagcagtttagggtctaccagaggctcatgggagcaggagcttagggtcccccagaggctcatggagcaggaacttagggtcctccagaggctcatgggagcagaagctttgggtcctccggggctcatgggaacaggaatttggggtgccccacaggcttatgagagcagtttagggtctgccagaggctcatgggagtaggagtttagggtcccccagaggctcatgggagcaggagtttagggtacccagaggctagtgggaacaggggtttggggtcccccagaggctcatgggagcagaaacttgagtcatccagaggctcatgggagcaggaatttggggttccccagaggctcatgggagcagtttaaggtcccccagaggctcatgggagctggagtttagggtcccccagtgaatcatgggagcaggagttttagggtcccccagaggctcatgggaacaggggtttggggtgccccagaggctcacgggagccaaggcagtcttcccagtgcggtagagcatagggtctgagcagattgaggccccaggactcctcctcatgtcccaccctactccagcccagcacccccctatgggctcttcgttgggggccgtttccaggctcctggggcccgaagctccaggcccatccgggattcgtctggcaacctccatggctacgtggctgagggtggagccaaggacatccgaggtgctgtggaggccgctcaccaggctttccctgggtaaggggtcacacgggaaagcccaagggtcatggtgtggcagagaggggagcctgcccacagcaattggtggactgggaggctgggcccccagggtaggggctgtggacagaggtggggtgtcaggagagccaagaccaggatgccaacccccactgtgcgctgtctgcccacagctgggcgggccagtccccaggagcccgggcagccctgctgtgggccctggcggctgcactggagcgccggaagtctaccctggcctcgaggctggagaggcagggagcggagctcaaggctgcggaggcggaggtggagctgagcgcaagacgacttcgggcgtggggggcccgggtgcaggcccaaggccacaccctgcaggtgaagggtctgtgggcacctgggccagctgggcaggcggggtggggctcagaccagaggctgtgagataacccaggcttggaattcgggcccagccccaccgccttccacggcaggatcatcagcaagtggcttcacctctccctacctcagttccctcatctgtacagtgagtgggcacacaacccgcctcgtagagtagcgttcgccagatgatggatagcgacccattcctggaccctaaaatcaatttagtgggctgttaggcggttctcacattgctgtaaagaaacacctgggggcgcagtggctcacgcctgtaatcccagtacttcgcgaggctgaggcaggcggatcacgaggtcaggagatcgagaccagcctgggcaacacggtgaaacctggtctctactaaaaatacaaaaaattagctgggtacggtggtgggcacctatagtcccagctacttaggaggctgaggcaggaaaattgcttgaacccaggaggcagtggttgcagtgagccgagatcgtgccactgcactccagcctgagtgacagagtgagaccttgtgtcacaaaaaaaaacaaaaaacaaaaaatacctggccagacatggtggctcacgcctgtaatcctagcactttgggaggcaaaagtgggtggatcgcttgaggccaggagtttgaaaccagcctgggcaacacagtgaaaccctgtctccacaaaaaatagataaattagctgggcatgttggcaccagtctccctctggcacccggctggagtgcagtgccacaatcatggctcactgcagcctcaacctccgaggtccaagtgagccccccacctcagcctcctgagtatctgggatcacaggcacacaccagcacacatgactgggtgtttttgtatttttgcagagacaagagactcgccatgttgcccaggctgatcttgaactcctagcttcaagtgatccgcctgtcttgacctcccaaagtgctgggattataggcgtgagccactgcgcctggccagggattacatttcaacatgagatttgggtgggacatctgaactatatcatgggccaaaccatatttctttctttcttttttttttttttttgagacagagtctcgctctgtcgcccaggctggagtgcagtggcgcaatctcggctcactgcaacctctgcatcccaggttcacgccattctcctgccccagccttccgagtggctgggactacaggtgcccaccaccacacccggctaattttttttttgtatatttagtagagacagggtttcaccatgttaggatggtctcgatctcctgacctcgtgatccgcccaccttggcctcccaaagtgctgggattacaggtgtgagccactacacccggccttcttttttttaaattttattttactttagttctgggatacatgtgcagaacgcgcaggcttgttacatagggatacacgtgccatggtggtttgctgcacctattgacctgccctctaagttccctccccttgcctcccaccccccaacaccatatttatttttaacaaaagaaaatagaccaggcgcagtggctcacgcctgtaatctcagcactttgcgaggccaaggtgggcagatcacaaggtcaagagatcgagaccatcctggctaacacagtgaaaccccgtctctactaaatatacaaaaaaaaaaaattagccgggcatggtggcaggtgcctgtagtctcagctactcaggagactgaggcaggagaatggcgtgaacccgggaggcggaggttgcagtgagccaagatcgcaccactgcactctagccagggcgacagagtgagagtccgtctcaaaaaaaaaaaaaaagaaaggcggttatgcaggatgtttctcaccgcccgaacccccgtggaatgattcactttgaccagcgtctgcgaaatggcaggggcttccacaatggtgcggtttgggccaacaccaagtgttggggagaatgtagaggaactggatctctcatttactgcgggcggggctcccctgccccacacgtgacccacctgtccctgcaggtagccgggctgagaggccctgtgctgcgcctgcgggagccgctgggtgtgctggctgtggtgtgtccggacgagtggcccctgcttgccttcgtgtccctgctggctcccgccctggcctacggcaacactgtggtcatggtgcccagtgcggcctgtcctctgctggccctggaggtctgccaggtatagccccctgccttcctgcctctcctccccgtagcctcagggcagcagaaaaaggcgccccaaagtcggcaggagcttgtctcttaccccaccctccgtggtacccatgctgcccccagccccagcacccaaaccttcactccttggggacccagtgcccattcttcaccctaggcctggggctttctcctccatgaccccccatccccttccctcccatgggcaccccctgaatgcccactccttgccctgcccccacggcctccccaacctttcactctctctatcccctaggacatggccaccgtgttcccagcaggcctggccaacgtggtgacaggagaccgggaccatctgacccgctgcctggccttgcaccaagacgtccaggccatgtggtatttcggatcagcccaggtgctctttgttctgttttgccatcttcagcatctcaaacttcaaacaggctcctccttttctggaaacacagccccgccctcttagaactcctgttggtaaggggagaaactccttgacaagaaggttttgaggccccgcccttaggactcaagttactaaggaagaggctgtccttagcaacaggggcctgagagctgcctagcccaaagacctcgtctcaggggacagcccgttgctaaggaccacaccctagatttggaggcccacagtctacgctttgtttttctattttgagacagaacctcaccctgtcacccagatgcaatcatacctcactgcagccgcaacctcccaggctcaagccaccgtcccacctcagcttcctgagtagctgggaccacaggcgtgcaccaccatgcccagctaatttttttcttttgcagagactggggtttctctatgttggccaggctgatcttgaactcctgggctcaagtgatctgcccgcctcggcctctcaaagtgctgggattacaggtgtgagccagcacaactggttgagtctactgtttttttttttattttattttattttattttattttttttgagacagagtctcactctgtcgcccaggctggaatacagtggcacgatcttggctcactgcaacctctgcctcccgggttcaagtgattctcctgcctcccaagtagctgagactacaggcactcaccgccatgcccagctaattgttgggggtttttttttagtagagatggggtttcgccatattggccaggctggtctcgaactcctgacctcgcgtgatctgcctgcctcagcctcccaaaatgctgagattacaggcgtgagccactgtgcccaaccaatttttactttttaaatggagacggggtctcgccatattgtccaggctggtctcaaactcctgaactcaagcgatcctcccgcctcagccaccccaagtgctgggattacaggagcgagtcaccgcgccccaccgtccactctcttaaacctcctgttggtaacagatgtgaacttgttgacaagagccttggggcccattcctttcaacatgggctgttaagggagaggaaattccctaaccactgggtccagtgcttttccctaccatctggaagttctgcttgggaggagctgggcattgtagctgcagagcctgggtccagcctgaagcccctcgtcagtaacagtcttcatcgcggaggaagcaggtgctcagcaacaagcctgcagaagtgcttacccccgtctcttcctcccctcagggttcccagtttgtcgagtgggcctcggcaggaaacctcaaaccggtgtgggcgagcaggggctgcccgcgggcctgggaccaggaggccgagggggcaggcccagagctggggctgcgagtggcgcggaccaaggccctgtggctgcctatgggggactgatgcctgagcgccacctactgcattttggacacctcacaccaaggggagatgcaccccacagacacctgggactttccccttctggttcctgtgtctcccaataaactctctgaccaaccctagctgtgcttctgcgagaagaaagggtgtagcaacttctggcagatatgaggcttttttcttttttttttttttttttgagacaacgtctggctctgtcacccaggctggagcgcagtggcacaatctcggcccactgcagcctcgacctctggggctcaagggatcctcatgcctcagcctcatatgtagctggggccacagacatgcaccaccacacctggctcgaggccattttagttctgaggttgagcagctcaggagccggctccagcacggtgctgtgtttgtgaaacagagaaaggggacccccgaggaccccagacagggccttaggactctcatatcttcttgtcttctccatctggtggctcttgctctctggttttctctaccttttcatggccccagaatccatatgcagtaaaggaactctctggaataaaattaaagtcctccta

SEQ ID NO: 5 - DNA, 401 bp, Homo sapiens
gctcagggtgttccagtaacttgggccagcgtggggcagcaaggagccatgggaggtgtgagagggaggaaaggagctgggaacaggtgtggagggtgcaggcaggtaggcaggtgggccaggagggaagaaggaggggaggccccagcgggtcccctaacatttcaaatctaacattttgtagtaattgctcgcacatayagtgccttggtaatgcaggcctcatgtggggctggcttgcttgctttatttttttttgagatggactctcactctgtcacccaggctggagtgcagtggcgggatcttggctcactgcaacctccacctcccaggttcaagcaattctcctgcctcagcctcccaagtagctgggattacaggcgcccgccaccatgcct

SEQ ID NO: 6 - DNA, 401 bp, Homo sapiens
gaatcacttgaacccgggaggcagaggttacggtgagctgagatcaggccactgcactccagcctagatgacagaacaagactccgtctcaaaaaaaaaaaaaaaagtctggttagtgatgacaataacaacaacaaaaataaatatatattcacatatgtacagtaagggtaacagctgatttttgttgagtgtttactrtgtaccagaaatcattttaaacatttgcacacaggcttacttgacctttacctaataaaccccgtgagatagcttggtagaattatcctcattttaaagatgggggctgggtgtggtggctcgcaccggtaatcccttcactttgggaggctgaggtggtagactcacttgagctcaagagttcgagatgagcctgggca

SEQ ID NO: 7 - DNA, 401 bp, Homo sapiens
ctcttctctccaccccccaaaccgttgccgcttgggaaccgatcttcctcctgctcccaacttgttctgcaccaccccatgaagttcagggcctatgtgttcccgtgtgtctacggaagaggaggcgtccatgtggacctctggcgttggccccatagcctgtgtcgccggaacgggggctggggggggtttgtggggtcsacgggggagggctctcatttgcaaatcaagacgcttgctctgccttggaccctcttctgtgtgttagtctcgtctctgagcatgcctgtttgtaaacgtgagactatccgggctctctttattcgggctctggagtgggggccatttctgtggctctttagttgagcaccccgattcccttgtgttctcccctttagggc

SEQ ID NO: 8 - DNA, 401 bp, Homo sapiens
tgttattagtcgggagttagaattctgggatagatggctgggcgcggaggctcatgttggtaatcccagcactctaggaggctaagggaggtggatcacgaggtcaggagaccaataacaacccatcctggctaacatggtgaaaccccgtctctactaaaaatacacaaaaaattagccgggtgtggtggtgggtgcctrtagtcccagctactcgcgaggctgaggcagaataatggtgtgaacccgggaggcggagcttgcagtgagccaaggtcgcgccactgcactccagcctgggcgacagagcgagactttgtcttaaaaaaaaaaaaaaaagaattctggggtagatgggatggttaggatagggctgagaattctgggatagatggttagga

SEQ ID NO: 9 - DNA, 401 bp, Homo sapiens
ctctcctcctgggctctaccttgctgatctcgaagccgaacacttcttcgaagtaggcgggctgggagatgagcagcaggtagaacgtccagctgcggcagaagttggccacgatgatggcatagactggcatagacgtgaagaagcgccgccagggagtgctaaacttctgtgggggcgaggggagggccgctaagacgsggagcggggctgagggcttctccgcgtcccttcagggaccacagtgtagttctgcagaaaccaaaggatcccgaccgcactgaaacttctatggacccaaaggcgcgggctacaccatgttgccgtggggacccagggatcctagcctcaaggtgacacttgagtgatgaaggggtcctggccctatggtagcctctcag

SEQ ID NO: 10 - DNA, 401 bp, Homo sapiens
ctcacggtcccaagtgtccagtcctctggcccccaaatcctggaagacccaagtacctagtttccagcccatcttgggcccaggcctccagctttgccatccccatgttcagataccctgaacctctcatgatcctaagtgtccagggacctggctcctatcctgctcagagcaccaggtctttagtctctacatatctcmgaaacattcatggttaaagcctggacaggactctgtcctgattgccccttgtggacccaggtttcctggtccccagacttacagcctccactgttaagccacaggtaggcaaagcgccagagacaagatggcttcatagagaagcccacctgagccagaatatactcagttttgctggaccagaacggacgggcaaggag

SEQ ID NO: 11 - DNA, 401 bp, Homo sapiens
agagaatttactacaacacacagcctgagaaccactcccccgacatgactaggtgcatgcatacatacataacacacatacacacacacacacacacagtgtgaggactcccactttgcctgccaccaattccactgagcacacacagactctcccaccaagcctctgtcttcccttaactcgtgtatccctccagggccygtagggtggtgccccaaggcttctcgcaggctcccctcacccagccagtgtcccagccttccgaatggtcaaaatgacccgccacatcctccactccatccttccttcattcatcctctgaatacatttatggatcacacccagggacacaggccaccctcactaatgatgggaggcactggcagaatttgttgagctcc

SEQ ID NO: 12 - DNA, 401 bp, Homo sapiens
cagctatttggggaggctgaggcaggagaatcacttgaacccgggaggtggaagttgcagtgagccaagattgcgccactgcactccagcctgggtgacagagtaagactctgactcaattaaaaaaaagaaagaaaaaaaaaaagagagaaggcgccgtctatgaaccaggaagcgccctcaccagacaccaagtctgcygtcgccttcatcttcgacttcccagcctccaaaactctgacataaatttctttctttctttctttcttttttttttttgaaacggagtctcactctgtcacccaggctggagtgcagtggcgcgatctcggctcactgcaagctccgcctcccgggttcacaccattcttctgcctcagctcccgagtagctgggactac

SEQ ID NO: 13 - DNA, 401 bp, Homo sapiens
actggcacccatccagcatgctgcagccaagggcctgtgtacttggagtttcctctgcccagaactagggggaccaactggtcctagtttgcccaggaccttcccagttttaacactgaaagtctcatggcctgggtagcccccgtcccaagtcatgggaacagcagacgttggtcaccctcacaatccctcatctccttyggattggtcactgctccagcgtcacccagagcgcagccttccctgcccgccttgtgtaaaatagaaccatggctcagcagggcccttcacacttctctgcactgcctggttccattgaactcacagccctctgacaggctgggcctcttcctaatgtgtttgtctatgaccggtcccctaggctctcaaatgtaagtgct

SEQ ID NO: 14 - DNA, 401 bp, Homo sapiens
tcccctatcactcttcacatcaccccctcccctatcactctcctcatcaccccctccccatcacttctcccccatcacctccctcccccatcaccccctcccccattaccccctctccccaatcaccaccccttccctatcactccccctcctcgccaccccctcccccattactccccctcctcaccacccccactcccycatcaccccctctccgtcatcacccctctcccacccctcacagatccaggcgctggaactggaccccaacctctaccgcgtgggacagagcaagatcttcttccgggctggggtcctggcccagctggaagaggagcgagacctgaaggtcaccgacatcatcgtctccttccaggcagctgcccggggatacctggctc

SEQ ID NO: 15 - DNA, 401 bp, Homo sapiens
gcgttcgcctgtgtgtgtcgatacctgcccgtgtgcatgcatgtctgcgtgcatcccctgtgtgtggatgtgtcattgtgtgtgcatctgtatgtatgcgtgtctgtgtctatatgtggcagtgttcatggtatctctgtgtccctctatgtgtgtacatgtgtatatatcagtgtgtgcatctacatgtgtacctgtgcrtgcaagtggatgtgtacatgagtgtagatacctgtgtgcatgcctgtgtgtgcgtgtctcaatgcttgccagcatctacgtgtgtccatgcatgtccctctgcacatggtgtgtgtgtacacactctgagtatacgatatggaggtgacaccagaggcccatcgtgtgtgaagccagtgatgaattctgttgtgtggccc

SEQ ID NO: 16 - DNA, 401 bp, Homo sapiens
aaggacagggcgacttccacagacctctttgccaaggtccaggggcaccctgtaatgctacccaacaggagagtagccaagacctgggggtcctccttacccttcagcttctagaggaggtcctgtttcctccaaggagctgagagatggaggaagaacccgccccgatagacgagtttcgtgtaagatcagggagagatkagaggaaagtgagacgtgttccccacaggagggggagcacgatgtggctgtcctctttggatgcagggaggagggaaaagagctaaggaggagaggggtggaaccacccctcctgtcgaaattttagtaccccacaccccaagatgtgaggaacctgagcatgaacgcggtagccatggggtgagcgcctggggccacgc

SEQ ID NO: 17 - DNA, 401 bp, Homo sapiens
cccaacaggagagtagccaagacctgggggtcctccttacccttcagcttctagaggaggtcctgtttcctccaaggagctgagagatggaggaagaacccgccccgatagacgagtttcgtgtaagatcagggagagattagaggaaagtgagacgtgttccccacaggagggggagcacgatgtggctgtcctctttgsatgcagggaggagggaaaagagctaaggaggagaggggtggaaccacccctcctgtcgaaattttagtaccccacaccccaagatgtgaggaacctgagcatgaacgcggtagccatggggtgagcgcctggggccacgctcagctgccccgagacctcccaacctcgcccggggtccagggaggacccactggtctagg

SEQ ID NO: 18 - DNA, 401 bp, Homo sapiens
ccaacccattcctcctgccccaagtcactgtactccttcccctttggccctgccctggcctctgtgggagccgccagccacatgtgaacagggagtactggaaaggcagggagtcctagcagagagtcctagcagagaggcctctaacaataaaacctgcactgggttctgaagactgagtacaataaagccgaacgagcygggcgtggtggctcaggcctgcaaccccagcactttgggaggccgaggcaggcggatcaccgagcccaagagttcgaagccagcctgggcaaaaaaaaaaaaaaaaaggaatttcatctgctttttttactcttttaacatggtgtctagaaaatttagaattccacgtggggcttgtgttgaatttgatggatgatgcc

SEQ ID NO: 19 - DNA, 401 bp, Homo sapiens
aaatggataatggtcgcgtgtatctccgtgttgttagaagatcacaggagctaagttgcttgctgtgtgtcgcttagggcctggtctgcagtcaaggttcattaaacaacacctgttcgtagcggccgttacctctgtatgcttctgcagcaggtgtttgttgagtatctgctttgttccagtatgtgcgtgcaagtgctrggtgcaggacatatagtgaaaaggtgacacagaagcttccccttcgcggtagctggaattcgtaggtaacacagagcagggtttctcatccttggccctagtgacactgtggacacagtcattccttgtagtgggggccgtcctgggtattgtaggatgtttaacatcatccttggcctctactcactagatgccggtag

SEQ ID NO: 20 - DNA, 401 bp, Homo sapiens
gaacaaggggtgctttagcggagtaacacggggcactgtgggtgataagtgaagacccctaaaccaagtctatgggagatccaggaagactttcctgaggaagggccttttggaaaagggttttgaaaggtgaataggagttcacccttaaagccacactccttccacagcatcaccacccagatctcctgaggagcatamcatgaggccctcatattttaaggaaacatgaaaaggagaaacaaccagctcccccaaagcccctgccttcctctgctctgaaattcccatcctctctcctttcccccaataaaggtccaagagggaacaggaggtgacggagctgaagaagactctggaggaggagactcgcatccacgaggcggcagtgcaggagctga

SEQ ID NO: 21 - DNA, 401 bp, Homo sapiens
ttttttttttggagacagagtcttgctctgtcacccaggttggagtgcaatggcgtgatctcggctcactgcaacctccacctcctgggttcaagggattctcctgcctcagcctcccgagtagctgggattacaggtgcctgccactatacacagctaatttttgtatttttagtagggacggggtttcaccatgttgcycaggccgatctcaaactcctgacctcaggtgatgcacctgcctcggcctcccaaagtgctgggattacagccgtgagccactggacccggccggacttcaacatatctttagcgggatacaattcaatgcatgacactcatagatttatgagtttatttctggactttcaattctattccactggattgcgtctatcctt

SEQ ID NO: 22 - DNA, 401 bp, Homo sapiens
ggcaagccacatgtaggagaatgaaactcgatccttatttctcaccttatacaaaaatcaactcacaaattcaatgtgaatttgcatcaaagacttaaatataagaccagaaaccataaaaattctagaagataacatcaaaaaaactcttctagatgttggcttaggcaacgagttcatgaccaagagcccaaaagcaartgcaacaaaaacaaaaataaacatatggatatatgggaacgatggctcacgcctgtaatcctggcactctgggaggcccagctgggcggatcttcaggtcaggagttccagaccaacatgacgaaaccccatctctattaaaaatacaaaaattggccaggtatggtgttggatgcctgtaatcccagctactcaggagg

SEQ ID NO: 23 - DNA, 401 bp, Homo sapiens
gtgcagtttttacctgtaatctctgggaggctgaggtgggaggatcacttgagctcaggagtttgagaccagccagggcaacatagcgaggcccctgctccacaaaatttttttatttttatttatttttatttttatttttttgagacagagtctcgctctgtcacccaggctggagtgcagtggcgcaatctcagctcrctgcaagctttgcctcccgggttcacgccattctcctacctcagtctccacagcagctgggactacaggcgcccaccaccacgcctggctaatttttttttttttttttttttttgcatttttagtagggacggggtttcaccgtgttagccaggatggtctcgatctgacctcgtgatccacctgcctcggcctcccaa